(19) United States
(12) Patent Application Publication
(10) Pub. No.: US 2023/0303512 A1
(43) Pub. Date: Sep. 28, 2023

(54) MODULATORS OF MYB-MEDIATED TRANSCRIPTION AND USES THEREOF

(71) Applicant: Massachusetts Institute of Technology, Cambridge, MA (US)

(72) Inventors: Angela N. Koehler, Belmont, MA (US); Becky Leifer, Malden, MA (US); Florian Kabinger, Vienna (AT); Brice Curtin, Cambride, MA (US); Jasmin Kruell, Watertown, MA (US)

(73) Assignee: Massachusetts Institute of Technology, Cambridge, MA (US)

(21) Appl. No.: 18/022,962

(22) PCT Filed: Aug. 26, 2021
(86) PCT No.: PCT/US2021/047701

§ 371 Date: Feb. 23, 2023

Related U.S. Application Data
(60) Provisional application No. 63/070,534, filed on Aug. 26, 2020.

(51) Int. Cl. C07D 401/04; C07D 401/14; A61P 35/00

(52) U.S. Cl. CPC C07D 401/04; C07D 401/14; A61P 35/00

(57) ABSTRACT

Provided herein are compounds that inhibit the function of MYB. In particular, provided are compounds that can inhibit MYB and/or disrupt the interaction between MYB and a protein associated with MYB, a protein whose dysregulation is implicated in cancer. Also provided are pharmaceutical compositions comprising the compounds, methods of inhibiting MYB function, and methods of treating cancer in a subject by administering a compound or composition described herein.

### RELATED APPLICATIONS

This application claims priority under 35 U.S.C. § 119(e) to U.S. Provisional application Ser. No. 63/070,534, filed Aug. 26, 2020, which is incorporated herein by reference in its entirety.

### BACKGROUND

The transcription factor myeloblastosis (MYB) is an essential regulator of normal hematopoiesis and a proto-oncogene that is widely deregulated in various cancers (e.g., leukemias, adenoid cystic carcinoma, and rhabdomyosarcoma). For example, high expression levels of MYB are characteristic of leukemias such as acute myeloid leukemia (AML), and MYB is essential for maintaining the leukemic phenotype. Since leukemic cells are addicted to high levels of MYB expression, there is an opportunity to inhibit MYB protein function (such as by direct inhibition of MYB or an MYB complex partner, possibly leading to protein degradation or a decrease in transcriptional activity) to impact survival of leukemic cells selectively over normal hematopoiesis. However, the MYB protein is difficult to target using small molecule probes due to its disordered conformational structure and the difficulty of finding selective probes.

### SUMMARY

The present disclosure provides new compounds, compositions, and methods for the treatment of cancer (e.g., leukemia).

In one aspect, provided herein are compounds of Formula (I):

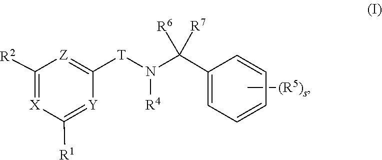

or a pharmaceutically acceptable salt, solvate, hydrate, polymorph, co-crystal, tautomer, stereoisomer, isotopically labeled derivative, or prodrug thereof, wherein:
X, Y, and Z are each independently N or CR³;
T is

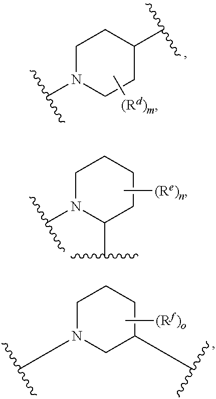

or —NR^{m}—(CR^{g}R^{h})₃—;
R¹, R², and R³ are each independently hydrogen, halogen, cyano, substituted or
unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};
R⁴ is hydrogen, substituted or unsubstituted alkyl, —C(═O)OR^{A}, —C(═O)R^{A}, —C(═O)N(R^{A})₂, or a nitrogen protecting group;
each R⁵ is independently —OR^{A}, —N(R^{A})₂, substituted or unsubstituted alkyl, substituted or unsubstituted carbocyclyl, substituted or unsubstituted aryl, substituted or unsubstituted heterocyclyl, or substituted or unsubstituted heteroaryl;
R⁶ and R⁷ are each independently hydrogen, halogen, or substituted or unsubstituted alkyl, or R⁶ and R⁷ together with the carbon to which they are attached form a carbonyl; each R^{A} is independently hydrogen, substituted or unsubstituted alkyl, substituted or unsubstituted heteroalkyl, substituted or unsubstituted acyl, an oxygen protecting group when bound to an oxygen, or a nitrogen protecting group when bound to a nitrogen, or two R^{A} groups are joined to form a substituted or unsubstituted heterocyclic ring or substituted or unsubstituted heteroaryl ring;
R^{d}, R^{e}, R^{f}, R^{g}, and R^{h} are each independently halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};
R^{m} is hydrogen, substituted or unsubstituted alkyl, —C(═O)OR^{A}, —C(═O)R^{A}, —C(═O)N(R^{A})₂, or a nitrogen protecting group;
m, n, and o are each independently 0, 1, 2, 3, 4, or 5; and
s is 0, 1, 2, 3, 4, or 5;
provided that the compound is not of formula:

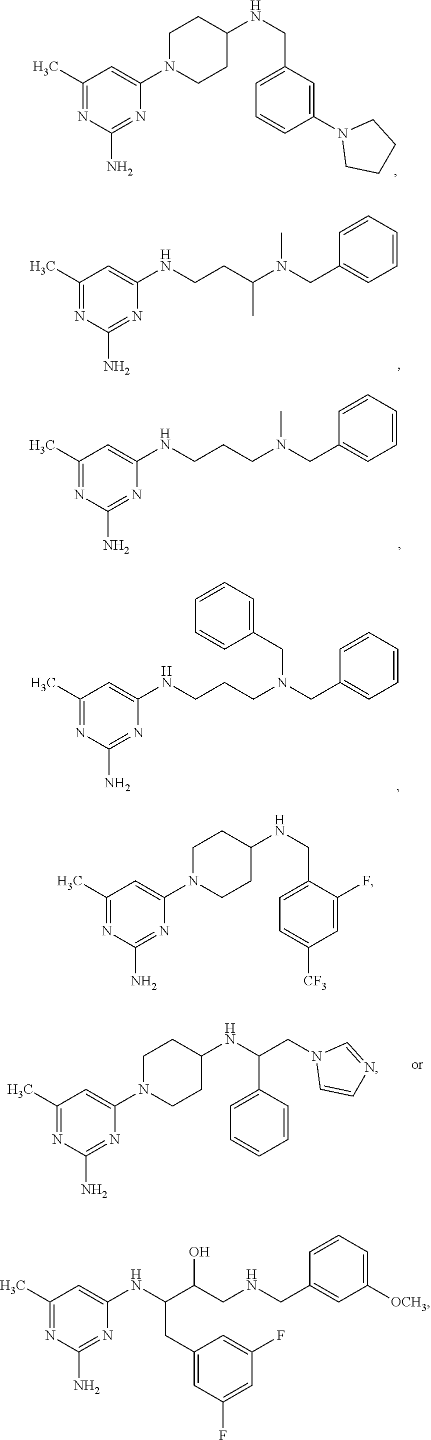

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of Formula (I-a), (I-b, (I-c), (I-d), (I-e), (I-f), (I-g), (I-h), (I-i), (I-j), (I-k), or (I-l):

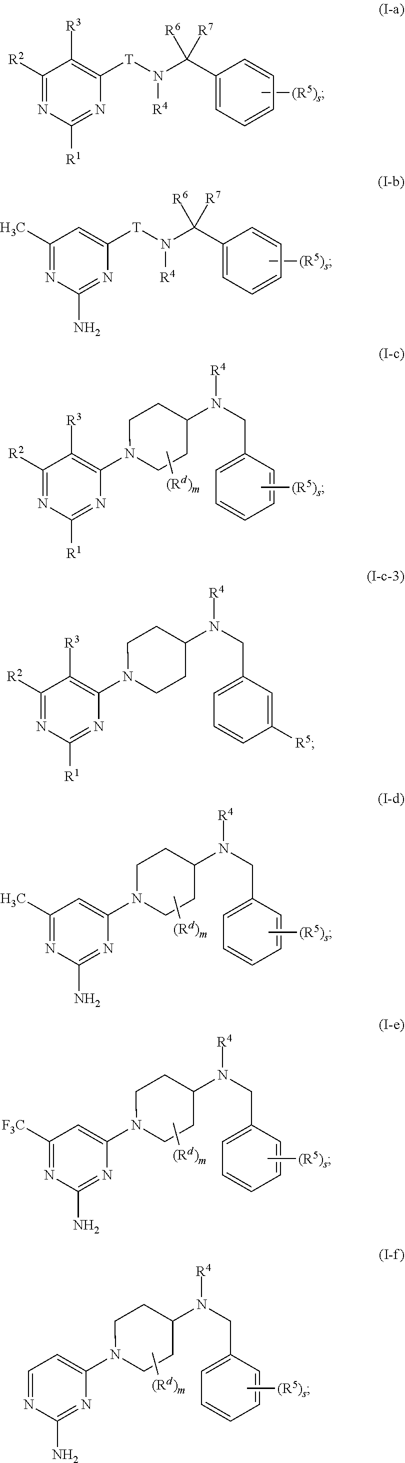

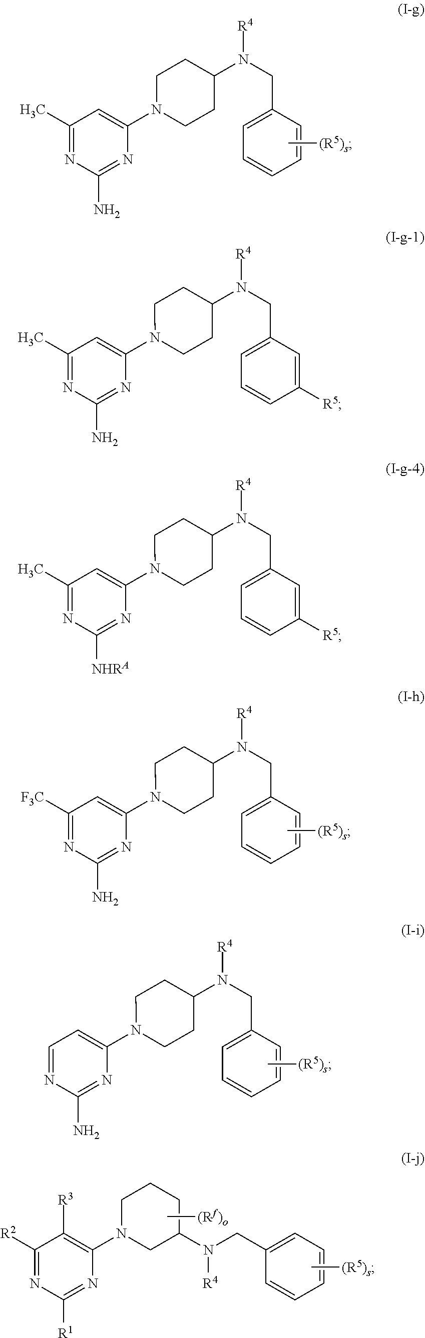

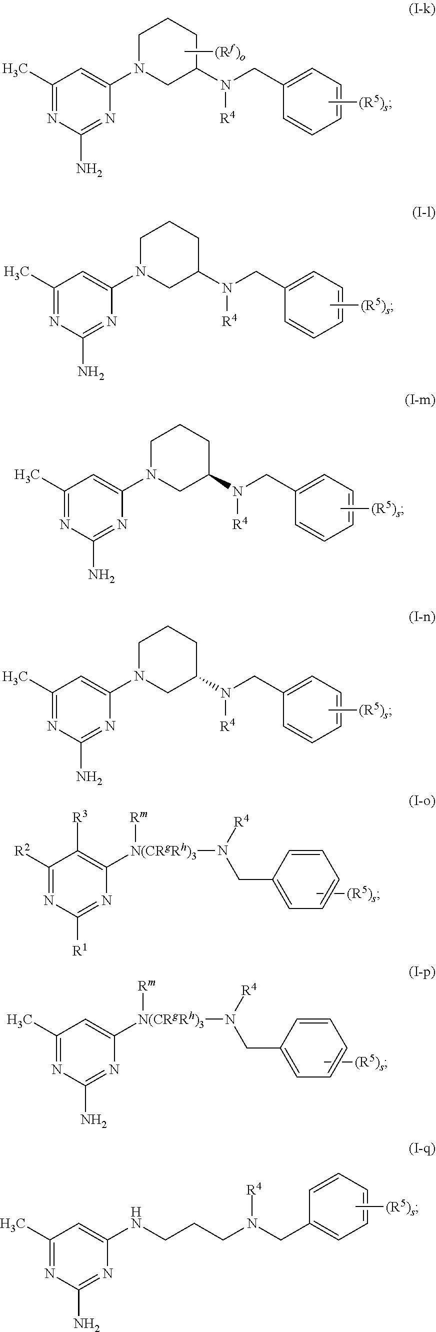

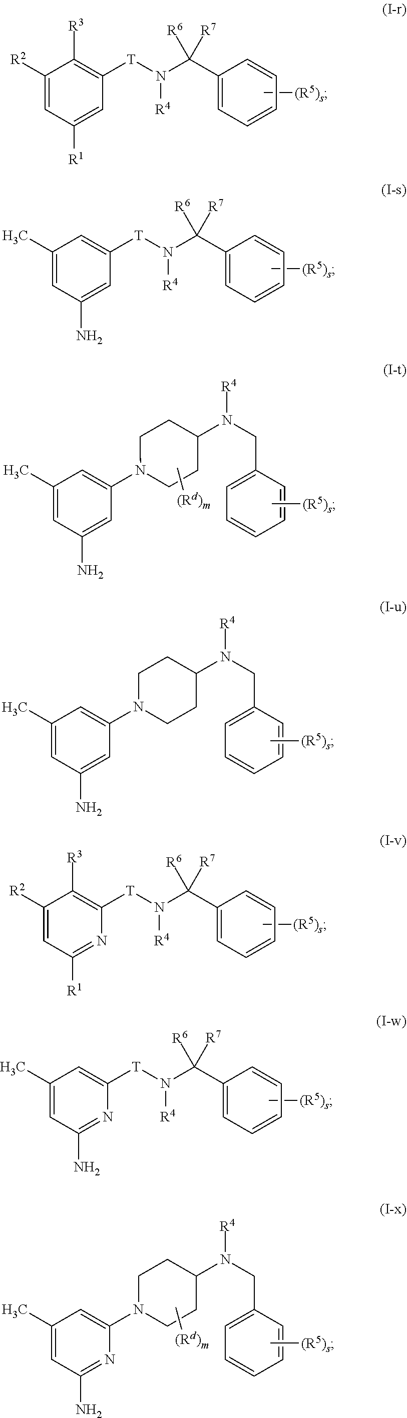

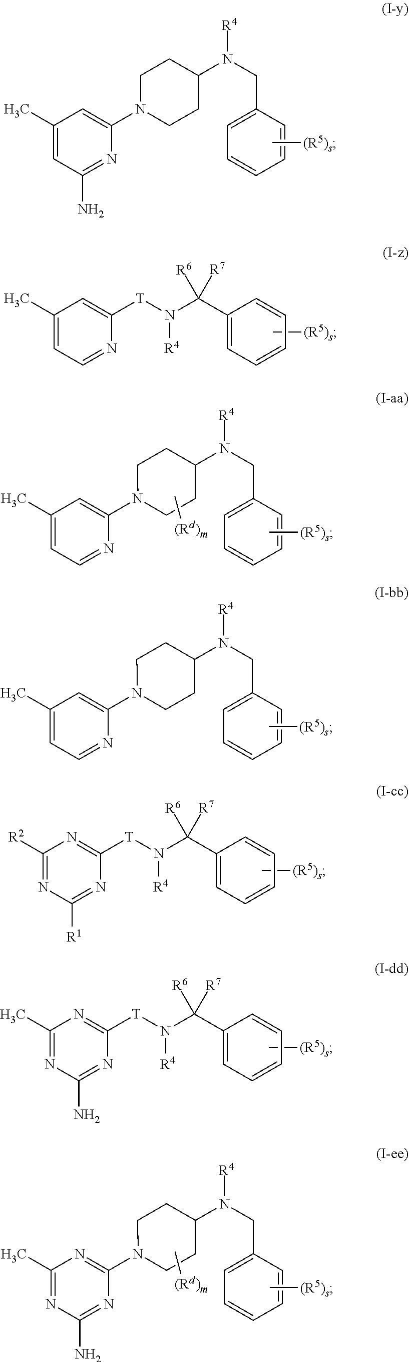

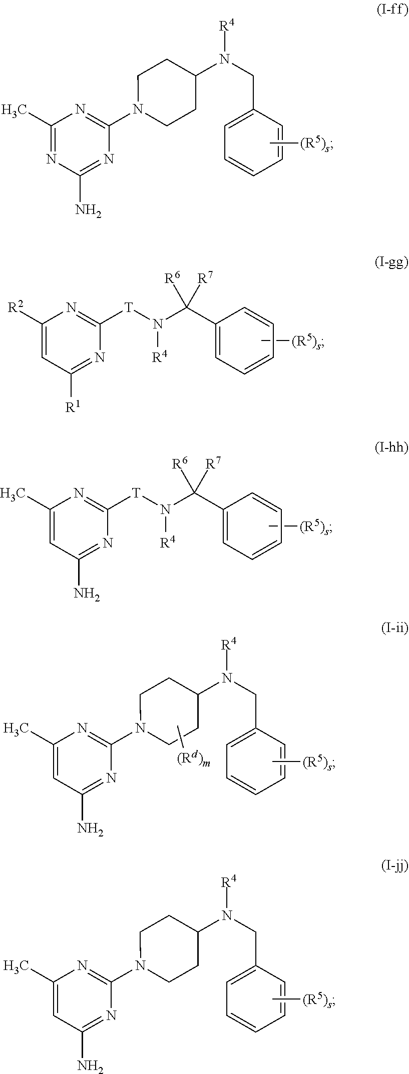

or a pharmaceutically acceptable salt, solvate, hydrate, polymorph, co-crystal, tautomer, stereoisomer, isotopically labeled derivative, or prodrug thereof.

Exemplary compounds of Formula (I) include, but are not limited to:

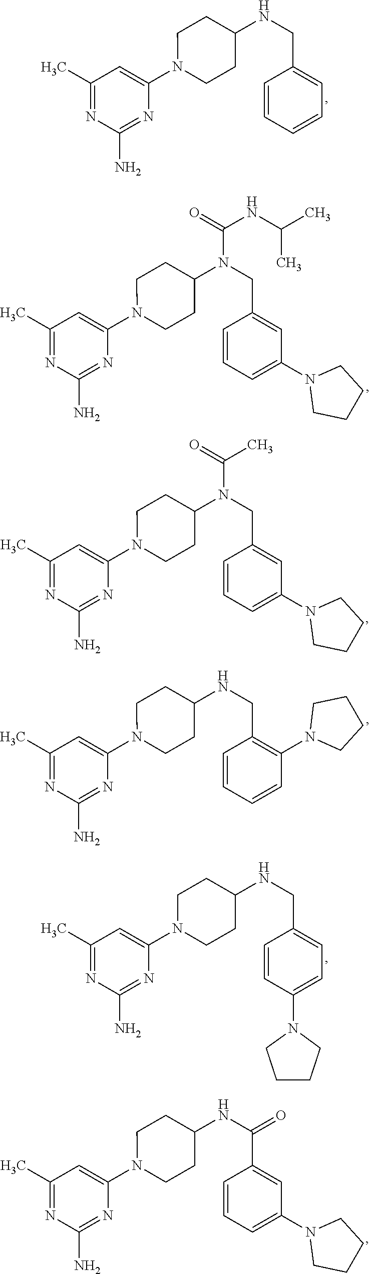

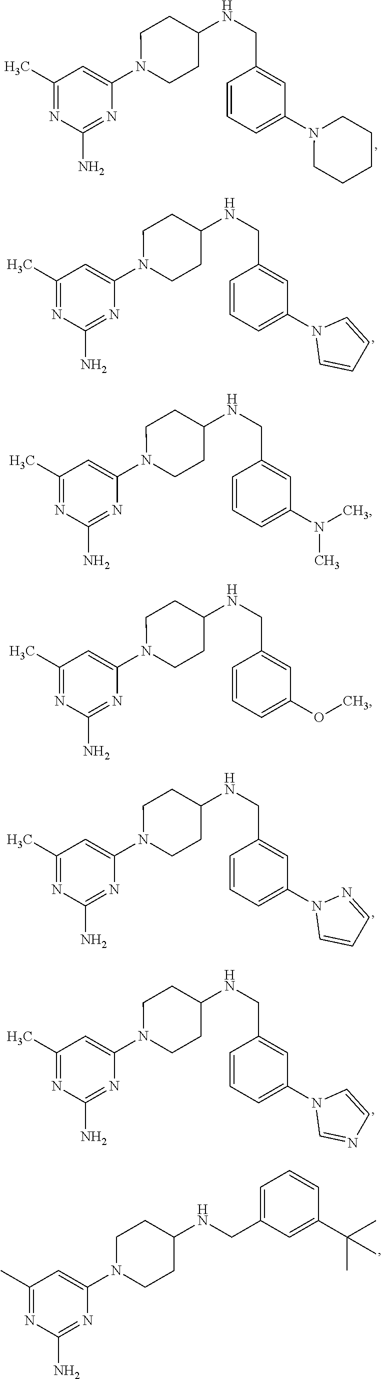

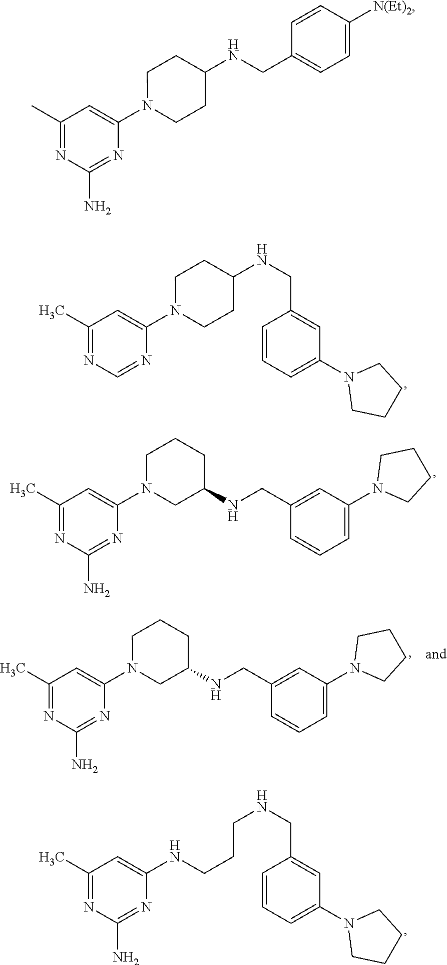

and pharmaceutically acceptable salts, solvates, hydrates, polymorphs, co-crystals, tautomers, stereoisomers, isotopically labeled derivatives, and prodrugs thereof.

Exemplary compounds of Formula (I) also include, but are not limited to:

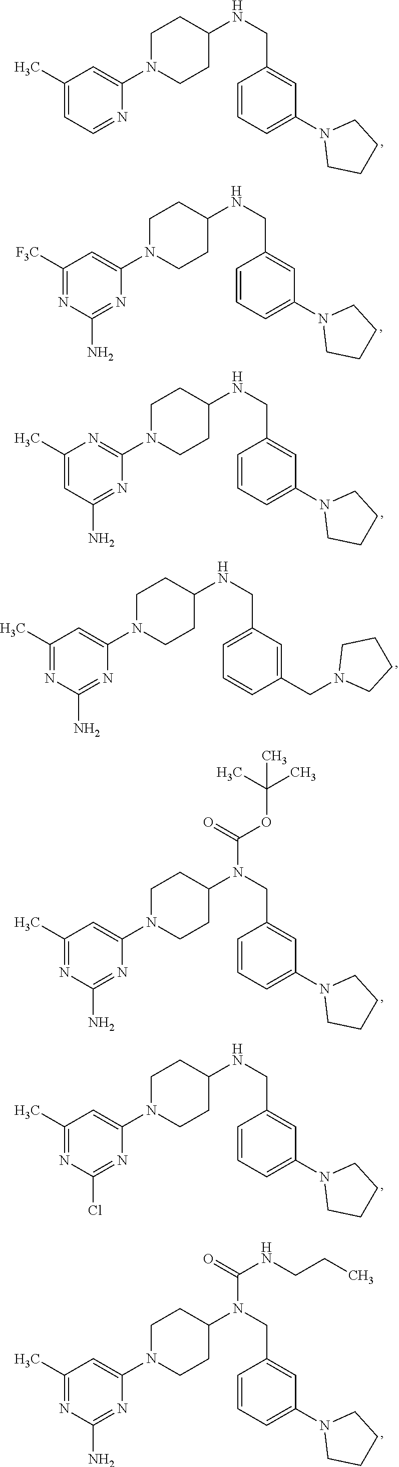

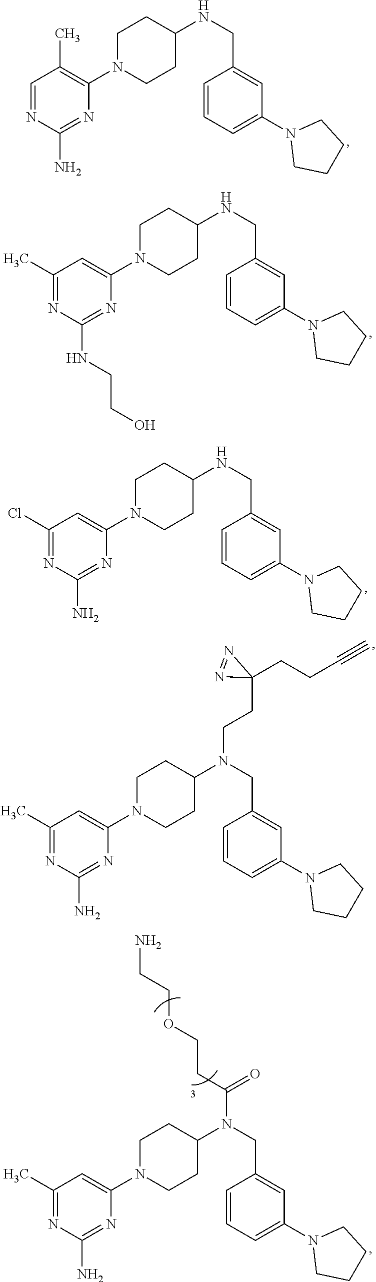

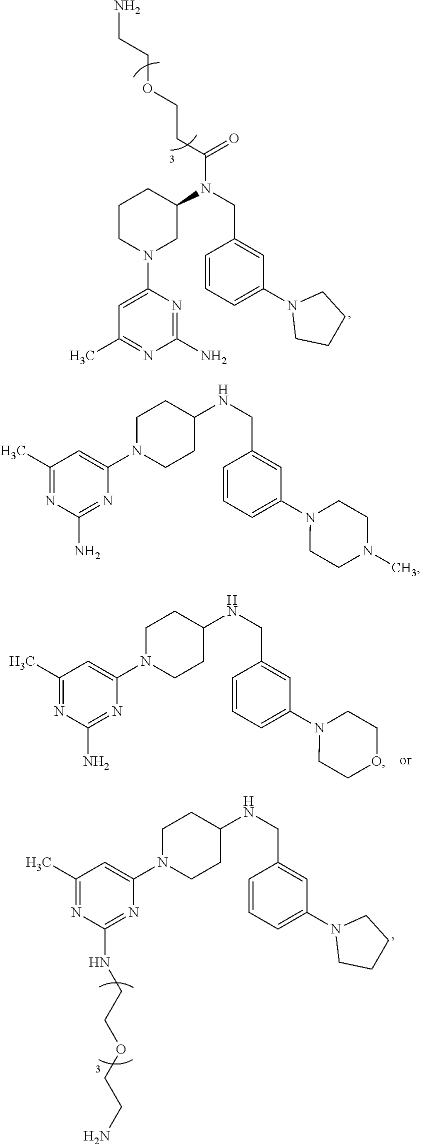

or a pharmaceutically acceptable salt, solvate, hydrate, polymorph, co-crystal, tautomer, stereoisomer, isotopically labeled derivative, or prodrug thereof.

Exemplary compounds of Formula (I) also include, but are not limited to:

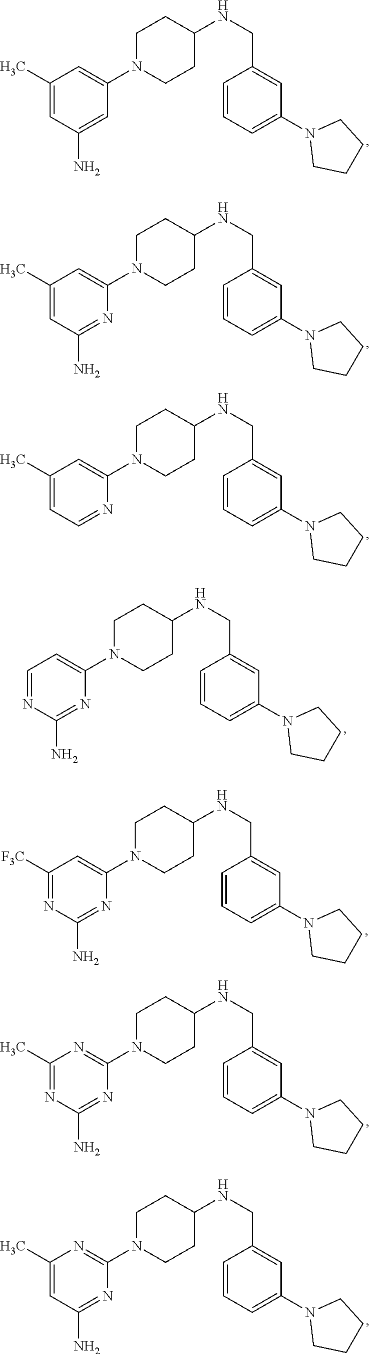

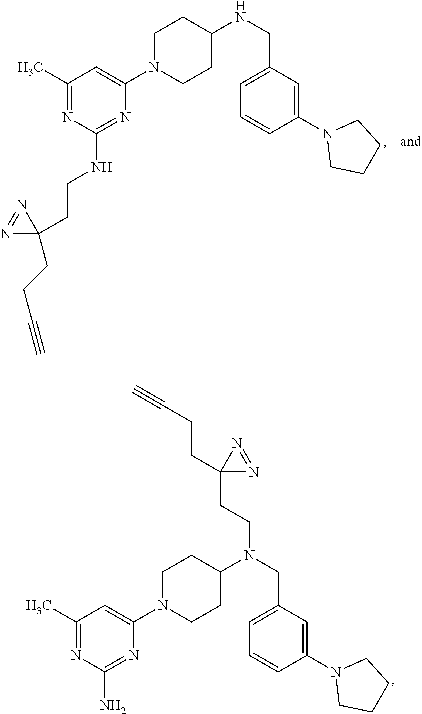 [SMILES: C#CCCC1(CCNc2nc(C)cc(N3CCC(NCc4cccc(N5CCCC5)c4)CC3)n2)N=N1 ; C#CCCC1(CCN(Cc2cccc(N3CCCC3)c2)C2CCN(c3cc(C)nc(N)n3)CC2)N=N1]

and pharmaceutically acceptable salts, solvates, hydrates, polymorphs, co-crystals, tautomers, stereoisomers, isotopically labeled derivatives, and prodrugs thereof.

Exemplary compounds of Formula (I) also include, but are not limited to:

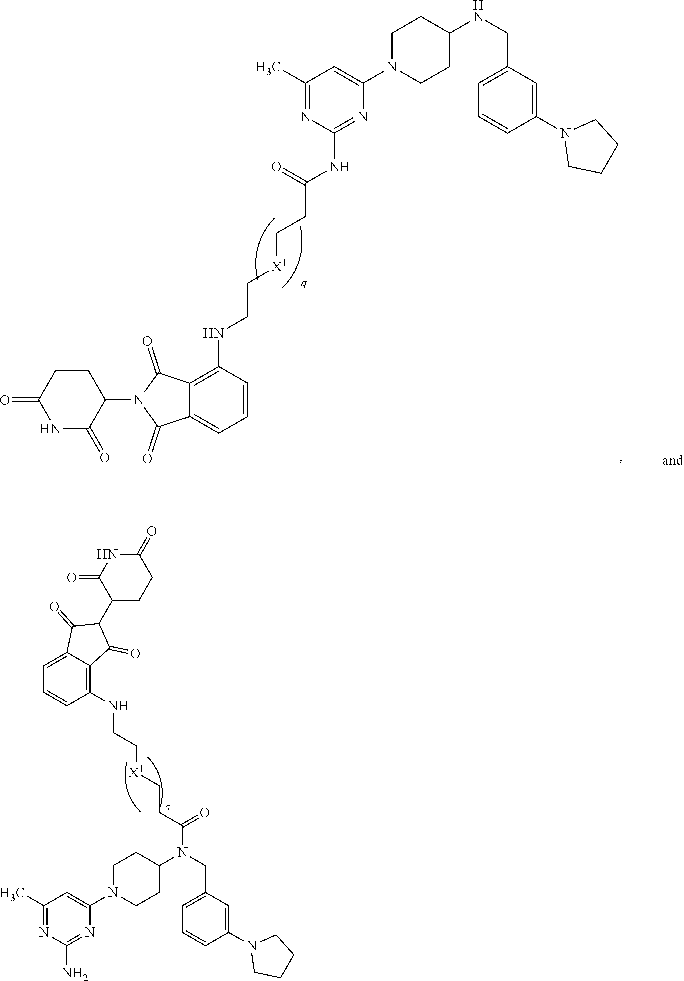

and pharmaceutically acceptable salts, solvates, hydrates, polymorphs, co-crystals, tautomers, stereoisomers, isotopically labeled derivatives, and prodrugs thereof, wherein X¹ is —O— or —CH₂—; and q is 1, 2, 3, or 4.

Exemplary compounds of Formula (I) also include, but are not limited to:

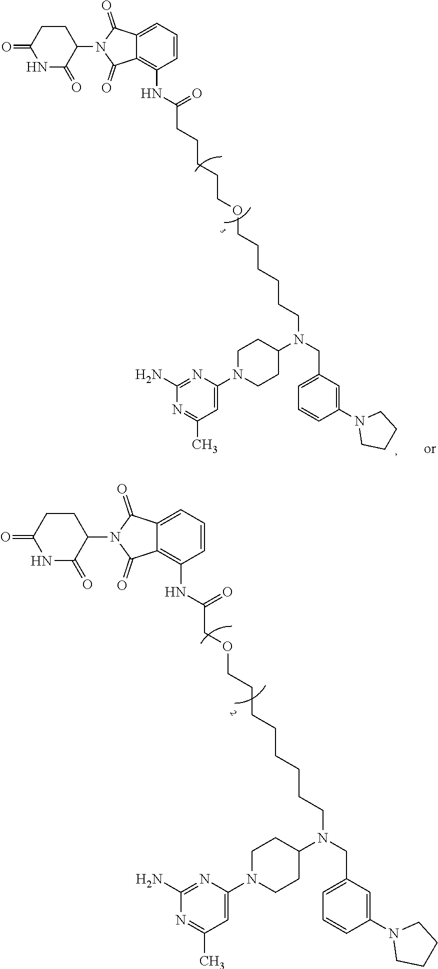

or a pharmaceutically acceptable salt, solvate, hydrate, polymorph, co-crystal, tautomer, stereoisomer, isotopically labeled derivative, or prodrug thereof.

In another aspect, provided are pharmaceutical compositions comprising a compound of Formula (I), or a pharmaceutically acceptable salt thereof, and a pharmaceutically acceptable excipient.

In another aspect, provided are methods of treating cancer in a subject in need thereof, the method comprising administering a compound of Formula (I), or a pharmaceutically acceptable salt thereof, or a pharmaceutical composition comprising a compound of Formula (I), to the subject. In certain embodiments, the cancer is a solid tumor or a hematological cancer. In certain embodiments, the cancer is a leukemia, a lymphoma, or multiple myeloma. In certain embodiments, the cancer is acute myeloid leukemia (AML) or acute lymphoblastic leukemia (ALL).

In another aspect, provided are methods of inhibiting MYB function, the method comprising contacting a compound of Formula (I), or a pharmaceutically acceptable salt, solvate, hydrate, polymorph, co-crystal, tautomer, stereoisomer, isotopically labeled derivative, or prodrug thereof, or a pharmaceutical composition comprising a compound of Formula (I), with MYB or a protein associated with MYB.

In another aspect, provided are kits comprising a compound of Formula (I), or a pharmaceutically acceptable salt thereof, or a pharmaceutical composition comprising a compound of Formula (I). In certain embodiments, the kit further comprises instructions for administration (e.g., human administration) and/or use.

The details of certain embodiments of the present disclosure are set forth in the Detailed Description of Certain Embodiments, as described below. Other features, objects, and advantages of the present disclosure will be apparent from the Definitions, Examples, Figures, and Claims.

### BRIEF DESCRIPTION OF THE DRAWINGS

FIG. 1A shows a schematic for in-cell BRET protein-protein interaction assays involving MYB. FIG. 1B is a plot showing that KI-TM-001 blocks the MYB-TAF12 interaction with an IC50 of 7.07 μM whereas a transcriptionally inactive analog does not perturb the interaction. FIGS. 1C-E are plots showing that KI-TM-001 treatment leads to a dose-dependent reduction of MYB protein in different cell lines.

FIG. 2 is a plot showing a dose curve of KI-TM-001, and its effect on MYB protein levels in Molt4 at the 6 hour timepoint.

FIG. 3A is a median area-under-the-curve (AUC) versus lineage plot suggesting the lineages most impacted by KI-TM-001 treatment include rhabdomysosarcoma, multiple myeloma, lymphoma, and leukemia. FIG. 3B is a plot showing Cell Titer Glo assay results for KI-TM-001 in several hematopoietic lines versus prostate and ovarian cell lines. MM1.S, a multiple myeloma line has a GI₅₀ of 2.01 μM.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein also include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ⁴C are within the scope of the disclosure. Such compounds are useful, for example, as analytical tools or probes in biological assays.

When a range of values is listed, it encompasses each value and sub-range within the range. For example “C₁₋₆ alkyl” is encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

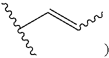

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂-8 alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), and the like. Exemplary C₃₋₁₀ carbocyclyl groups include, without limitation, the aforementioned C₃_s carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds and/or one or more carbon-heteroatom double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{aa};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{f})₂, —N(R^{f})₂, —N(R^{f})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 5-10 membered heteroaryl; or two geminal R⁹⁹ substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{cc})₃, —OP(R^{cc})₂, —OP(R^{cc})₃⁺X⁻, —OP(OR^{cc})₂, —OP(OR^{cc})₃⁺X⁻, —OP(═O)(R^{cc})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{cc} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{cc} and R^{bb} are as defined herein.

The term “silyl” refers to the group —Si(R^{aa})₃, wherein R^{cc} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, Rb, R^{cc}, and R^{dd} are as defined herein.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{ee}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and Rad are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{cc})₃, —P(R^{cc})₂, —P(R^{cc})₃⁺X⁻, —P(OR^{c})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “leaving group” is given its ordinary meaning in the art of synthetic organic chemistry and refers to an atom or a group capable of being displaced by a nucleophile. See, for example, Smith, March's Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{cc})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and claims. The present disclosure is not limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “tissue” refers to any biological tissue of a subject (including a group of cells, a body part, or an organ) or a part thereof, including blood and/or lymph vessels, which is the object to which a compound, particle, and/or composition of the present disclosure is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for MYB binding and/or disrupting the interaction of MYB with another protein (e.g., TAF12, p300, NFIB). In certain embodiments, a therapeutically effective amount is an amount sufficient for treating a cancer.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development.

Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenström's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “leukemia” refers to broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which arises from transformed cells of mesenchymal origin. Sarcomas are malignant tumors of the connective tissue and are generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

### DETAILED DESCRIPTION OF CERTAIN EMBODIMENTS

Provided herein are compounds that inhibit MYB function. The compounds may bind MYB or may bind a protein that associates with or interacts with MYB (e.g., NFIB, p300, TAF12). The compounds may disrupt interactions or associations between MYB and another protein (e.g., NFIB, p300, TAF12). In one aspect, the disclosure provides compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. The compounds are useful for the treatment of diseases associated with MYB (e.g., cancer) in a subject in need thereof.

### Compounds

In one aspect, disclosed is a compound of Formula (I):

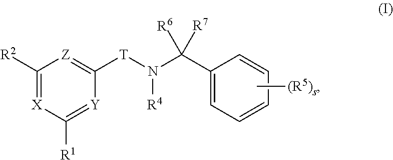

or a pharmaceutically acceptable salt, co-crystal, tautomer, stereoisomer, solvate, hydrate, polymorph, isotopically enriched derivative, or prodrug thereof, wherein:
X, Y, and Z are each independently N or CR³;
T is

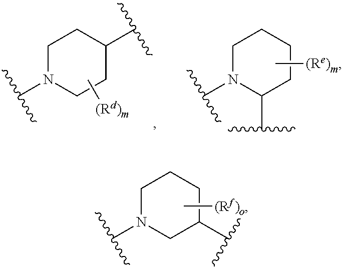

or —NR^{m}—(CR^{g}R^{h})₃—;
R¹, R², and R³ are each independently hydrogen, halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};
R⁴ is hydrogen, substituted or unsubstituted alkyl, —C(═O)OR^{A}, —C(═O)R^{A}, —C(═O)N(R^{A})₂, or a nitrogen protecting group;
each R⁵ is independently —OR^{A}, —N(R^{A})₂, substituted or unsubstituted alkyl, substituted or unsubstituted carbocyclyl, substituted or unsubstituted aryl, substituted or unsubstituted heterocyclyl, or substituted or unsubstituted heteroaryl;
R⁶ and R⁷ are each independently hydrogen, halogen, or substituted or unsubstituted alkyl, or R⁶ and R⁷ together with the carbon to which they are attached form a carbonyl;
each R^{A} is independently hydrogen, substituted or unsubstituted alkyl, substituted or unsubstituted heteroalkyl, substituted or unsubstituted acyl, an oxygen protecting group when bound to an oxygen, or a nitrogen protecting group when bound to a nitrogen, or two R^{A} groups are joined to form a substituted or unsubstituted heterocyclic ring or substituted or unsubstituted heteroaryl ring;
R^{d}, R^{e}, R^{f}, R^{g}, and R^{h} are each independently halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};
R^{m} is hydrogen, substituted or unsubstituted alkyl, —C(═O)OR^{A}, —C(═O)R^{A}, —C(═O)N(R^{A})₂, or a nitrogen protecting group;
m, n, and o are each independently 0, 1, 2, 3, 4, or 5; and
s is 0, 1, 2, 3, 4, or 5;
provided that the compound is not of formula:

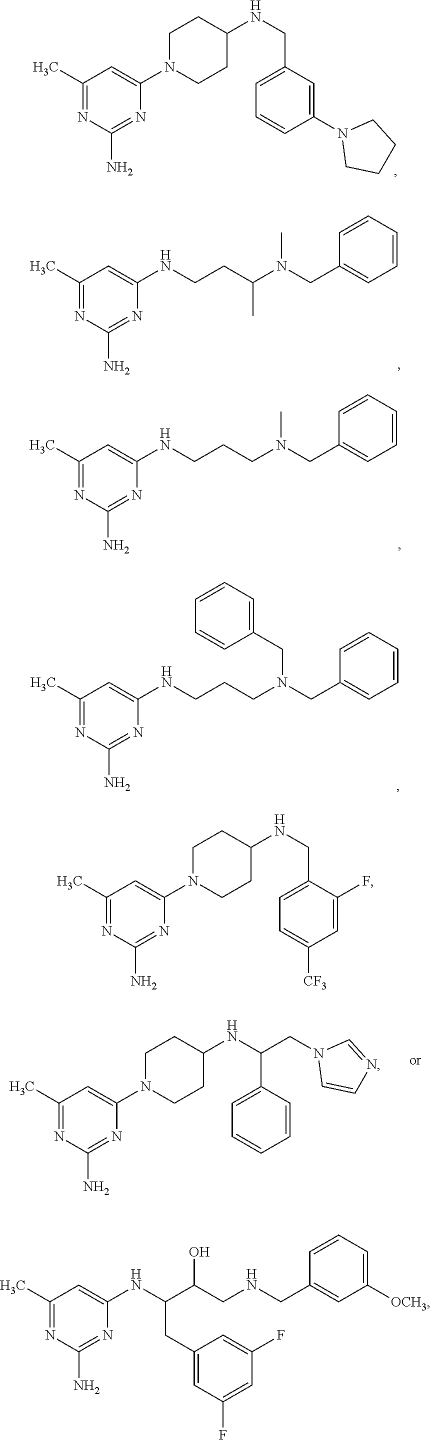

or a pharmaceutically acceptable salt, co-crystal, tautomer, stereoisomer, solvate, hydrate, polymorph, isotopically enriched derivative, or prodrug thereof.
X, Y, and Z

As described herein, X, Y, and Z are each independently N or CR³. In certain embodiments, X is CR³. In certain embodiments, X is CH. In certain embodiments, X is N. In certain embodiments, Y is CR³. In certain embodiments, Y is CH. In certain embodiments, Y is N. In certain embodiments, Z is CR³. In certain embodiments, Z is CH. In certain embodiments, Z is N.

In certain embodiments, X, Y, and Z are each CR³. In certain embodiments, X, Y, and Z are each CH. In certain embodiments, X and Z are each CR³; and Y is N. In certain embodiments, X and Z are each CH; and Y is N. In certain embodiments, X and Y are each N; and Z is CR³. In certain embodiments, X and Y are each N; and Z is CH. In certain embodiments, X, Y, and Z are each N. In certain embodiments, Y and Z are each N; and X is CR³. In certain embodiments, Y and Z are each N; and X is CH.

R¹, R², and R³

As described herein, R¹, R², and R³ are each independently hydrogen, halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A}.

In certain embodiments, R¹, R², and R³ are each independently hydrogen, halogen, cyano, substituted or unsubstituted alkyl, —NO₂, —OR^{A}, —N(R^{A})₂, or —NR^{A}C(═O)R^{A}. In certain embodiments, R¹, R², and R³ are each independently hydrogen, halogen, cyano, substituted or unsubstituted alkyl, —NO₂, —OR^{A}, —N(R^{A})₂, or —NR^{A}C(═O)R^{A}. In certain embodiments, R¹, R², and R³ are each independently hydrogen, substituted or unsubstituted alkyl, —NR^{A}C(═O)R^{A}, or —N(R^{A})₂. In certain embodiments, R¹, R², and R³ are each independently hydrogen, halogen, cyano, substituted or unsubstituted alkyl, —NO₂, —OR^{A}, or —N(R^{A})₂. In certain embodiments, R¹, R², and R³ are each independently hydrogen, substituted or unsubstituted alkyl, or —N(R^{A})₂. In certain embodiments, R¹, R², and R³ are each independently hydrogen, substituted or unsubstituted C₁₋₄ alkyl, or —N(R^{A})₂. In certain embodiments, R¹, R², and R³ are each independently hydrogen, substituted or unsubstituted C₁₋₄ alkyl, or —N(R^{A})₂, wherein R^{A} is hydrogen or unsubstituted alkyl. In certain embodiments, R¹, R², and R³ are each independently hydrogen, unsubstituted C₁₋₄ alkyl, or —N(R^{A})₂, wherein R^{A} is hydrogen or unsubstituted alkyl. In certain embodiments, R¹, R², and R³ are each independently hydrogen, substituted or unsubstituted C₁₋₄ alkyl, or —NH₂. In certain embodiments, R¹, R², and R³ are each independently hydrogen, unsubstituted C₁₋₄ alkyl, or —NH₂. In certain embodiments, R¹, R², and R³ are each independently hydrogen, unsubstituted C₁₋₃ alkyl, or —NH₂. In certain embodiments, R¹, R², and R³ are each independently hydrogen, unsubstituted C₁₋₂ alkyl, or —NH₂. In certain embodiments, R¹, R², and R³ are each independently hydrogen, methyl, or —NH₂.

In certain embodiments, R¹ is hydrogen, —N(R^{A})₂, or —NO₂. In certain embodiments, R¹ is —N(R^{A})₂ or —NO₂. In certain embodiments, R¹ is hydrogen or —N(R^{A})₂. In certain embodiments, R¹ is hydrogen or —N(R^{A})₂, wherein R^{A} is hydrogen or unsubstituted alkyl. In certain embodiments, R¹ is hydrogen or —N(R^{A})₂, wherein R^{A} is hydrogen or unsubstituted C₁₋₄ alkyl. In certain embodiments, R¹ is hydrogen or —NH₂. In certain embodiments, R¹ is hydrogen. In certain embodiments, R¹ is —N(R^{A})₂. In certain embodiments, R¹ is —N(R^{A})₂, wherein R^{A} is hydrogen or unsubstituted alkyl. In certain embodiments, R¹ is —N(R^{A})₂, wherein R^{A} is hydrogen or unsubstituted C₁₋₄ alkyl. In certain embodiments, R¹ is —NH₂.

In certain embodiments, R¹ is —N(R^{A})₂ or halogen. In certain embodiments, R¹ is —N(R^{A})₂, and each R^{A} is independently hydrogen, substituted or unsubstituted alkyl, or substituted or unsubstituted heteroalkyl. In certain embodiments, R¹ is —N(R^{A})₂, and each R^{A} is independently hydrogen, substituted alkyl, or substituted or unsubstituted heteroalkyl. In certain embodiments, R¹ is —NH₂.

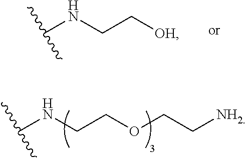

In certain embodiments, R¹ is —Cl, —NH₂,

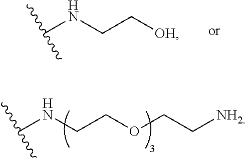

In certain embodiments, R¹ is —Cl. In certain embodiments, R¹ is

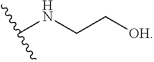 [SMILES: *NCCO]

In certain embodiments, R¹ is

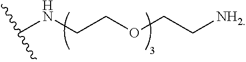

In certain embodiments, R¹ is —N(R^{A})₂, and each R^{A} is independently hydrogen or substituted or unsubstituted alkyl, wherein the substituted alkyl comprises formula

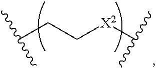

wherein X² is heterocyclyl. In certain embodiments, R¹ is —NHR^{A}, and R^{A} is of formula

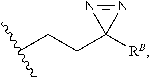

wherein R^{B} is substituted alkyl. In certain embodiments, R¹ is of formula

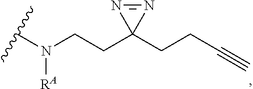

In certain embodiments, R¹ is —NR^{A}C(═O)R^{A}. In certain embodiments, R¹ is —NR^{A}C(═O)R^{A}, and each R^{A} is independently hydrogen, substituted heteroalkyl, or substituted alkyl, wherein the substituted heteroalkyl and alkyl comprise at least one instance of formula

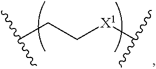

wherein X¹ is —O— or —CH₂—. In certain embodiments, R¹ is

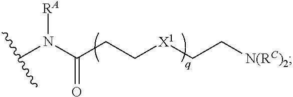

wherein: X¹ is —O— or —CH₂—; q is 1, 2, 3, or 4; and each R^{C} is independently hydrogen, substituted or unsubstituted alkyl, substituted or unsubstituted heteroalkyl, substituted or unsubstituted acyl, substituted or unsubstituted aryl, substituted or unsubstituted heteroaryl, or a nitrogen protecting group. In certain embodiments, R¹ is

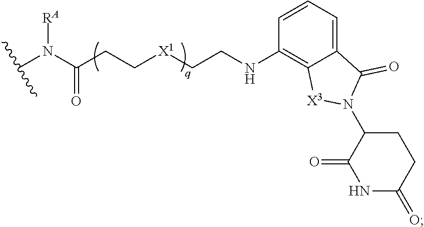

wherein: X¹ is —O— or —CH₂—; q is 1, 2, 3, or 4; and X³ is C═O or CH₂. In certain embodiments, R¹ is

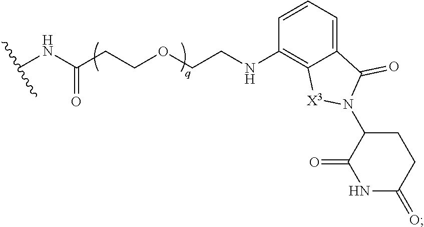

wherein: q is 1, 2, 3, or 4; and X³ is C═O or CH₂. In certain embodiments, R¹ is

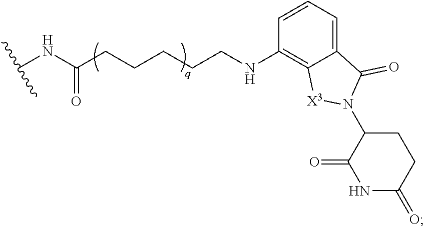

wherein: q is 1, 2, 3, or 4; and X³ is C═O or CH₂.

In certain embodiments, R² is hydrogen or substituted or unsubstituted alkyl. In certain embodiments, R² is hydrogen, unsubstituted alkyl, or haloalkyl. In certain embodiments, R² is hydrogen, unsubstituted C₁₋₃ alkyl, or C₁₋₃ haloalkyl. In certain embodiments, R² is hydrogen, —CH₃, or —CF₃. In certain embodiments, R² is hydrogen. In certain embodiments, R² is —CF₃.

In certain embodiments, R² is substituted or unsubstituted alkyl. In certain embodiments, R² is substituted or unsubstituted C₁₋₄ alkyl. In certain embodiments, R² is substituted or unsubstituted C₁₋₃ alkyl. In certain embodiments, R² is substituted or unsubstituted C₁₋₂ alkyl. In certain embodiments, R² is substituted or unsubstituted methyl. In certain embodiments, R² is unsubstituted alkyl. In certain embodiments, R² is unsubstituted C₁₋₄ alkyl. In certain embodiments, R² is unsubstituted C₁₋₃ alkyl. In certain embodiments, R² is unsubstituted C₁₋₂ alkyl. In certain embodiments, R² is —CH₃.

In certain embodiments, R³ is halogen or hydrogen. In certain embodiments, R³ is fluoro or hydrogen. In certain embodiments, R³ is hydrogen. In certain embodiments, R³ is hydrogen or substituted or unsubstituted alkyl. In certain embodiments, R³ is hydrogen or substituted or unsubstituted C₁₋₄ alkyl. In certain embodiments, R³ is hydrogen or —CH₃. In certain embodiments, R³ is substituted or unsubstituted alkyl. In certain embodiments, R³ is substituted or unsubstituted C₁₋₄ alkyl. In certain embodiments, R³ is —CH₃.

In certain embodiments, R¹ is hydrogen, —N(R^{A})₂, or —NO₂; R² is substituted or unsubstituted alkyl; and R³ is hydrogen. In certain embodiments, R¹ is hydrogen or —N(R^{A})₂; R² is substituted or unsubstituted alkyl; and R³ is hydrogen. In certain embodiments, R¹ is —N(R^{A})₂; R² is substituted or unsubstituted alkyl; and R³ is hydrogen.

In certain embodiments, R¹ is —N(R^{A})₂; R² is substituted or unsubstituted C₁₋₄ alkyl; and R³ is hydrogen. In certain embodiments, R¹ is —N(R^{A})₂; R² is unsubstituted alkyl; and R³ is hydrogen. In certain embodiments, R¹ is —N(R^{A})₂; R² is unsubstituted C₁₋₄ alkyl; and R³ is hydrogen. In certain embodiments, R¹ is —N(R^{A})₂; R² is unsubstituted C₁₋₃ alkyl; and R³ is hydrogen. In certain embodiments, R¹ is —N(R^{A})₂; R² is unsubstituted C₁₋₂ alkyl; and R³ is hydrogen. In certain embodiments, R¹ is —N(R^{A})₂; R² is unsubstituted methyl; and R³ is hydrogen.

In certain embodiments, R¹ is —N(R^{A})₂, wherein R^{A} is hydrogen or unsubstituted alkyl; R² is substituted or unsubstituted C₁₋₄ alkyl; and R³ is hydrogen. In certain embodiments, R¹ is —N(R^{A})₂, wherein R^{A} is hydrogen or unsubstituted alkyl; R² is unsubstituted alkyl; and R³ is hydrogen. In certain embodiments, R¹ is —N(R^{A})₂, wherein R^{A} is hydrogen or unsubstituted alkyl; R² is unsubstituted C₁₋₄ alkyl; and R³ is hydrogen. In certain embodiments, R¹ is —N(R^{A})₂, wherein R^{A} is hydrogen or unsubstituted alkyl; R² is unsubstituted C₁₋₃ alkyl; and R³ is hydrogen. In certain embodiments, R¹ is —N(R^{A})₂, wherein R^{A} is hydrogen or unsubstituted alkyl; R² is unsubstituted C₁₋₂ alkyl; and R³ is hydrogen. In certain embodiments, R¹ is —N(R^{A})₂, wherein R^{A} is hydrogen or unsubstituted alkyl; R² is unsubstituted methyl; and R³ is hydrogen.

In certain embodiments, R¹ is —NH₂; R² is substituted or unsubstituted C₁₋₄ alkyl; and R³ is hydrogen. In certain embodiments, R¹ is —NH₂; R² is unsubstituted alkyl; and R³ is hydrogen. In certain embodiments, R¹ is —NH₂; R² is unsubstituted C₁₋₄ alkyl; and R³ is hydrogen. In certain embodiments, R¹ is —NH₂; R² is unsubstituted C₁₋₃ alkyl; and R³ is hydrogen. In certain embodiments, R¹ is —NH₂; R² is unsubstituted C₁₋₂ alkyl; and R³ is hydrogen. In certain embodiments, R¹ is —NH₂; R² is unsubstituted methyl; and R³ is hydrogen.

In certain embodiments, R¹ is —N(R^{A})₂ or halogen; R² is substituted or unsubstituted C₁₋₄ alkyl; and R³ is hydrogen. In certain embodiments, R¹ is —N(R^{A})₂, wherein each R^{A} is independently hydrogen, substituted or unsubstituted alkyl, or substituted or unsubstituted heteroalkyl; R² is substituted or unsubstituted C₁₋₄ alkyl; and R³ is hydrogen. In certain embodiments, R¹ is —N(R^{A})₂, wherein each R^{A} is independently hydrogen, substituted alkyl, or substituted or unsubstituted heteroalkyl; R² is substituted or unsubstituted C₁₋₄ alkyl; and R³ is hydrogen. In certain embodiments, R¹ is —NH₂,

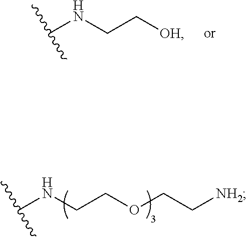

R² is substituted or unsubstituted C₁₋₄ alkyl; and R³ is hydrogen. In certain embodiments, R¹ is —Cl, —NH₂,

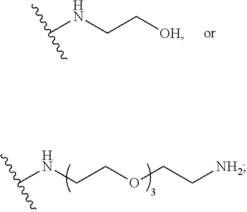

R² is substituted or unsubstituted C₁₋₄ alkyl; and R³ is hydrogen. In certain embodiments, R¹ is —Cl; R² is substituted or unsubstituted C₁₋₄ alkyl; and R³ is hydrogen. In certain embodiments, R¹ is

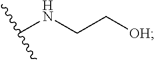 [SMILES: *NCCO]

R² is substituted or unsubstituted C₁₋₄ alkyl; and R³ is hydrogen. In certain embodiments, R¹ is

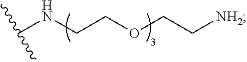

R² is substituted or unsubstituted C₁₋₄ alkyl; and R³ is hydrogen.

In certain embodiments, R¹ is —N(R^{A})₂ or halogen; R² is unsubstituted C₁₋₄ alkyl; and R³ is hydrogen. In certain embodiments, R¹ is —N(R^{A})₂, wherein each R^{A} is independently hydrogen, substituted or unsubstituted alkyl, or substituted or unsubstituted heteroalkyl; R² is unsubstituted C₁₋₄ alkyl; and R³ is hydrogen. In certain embodiments, R¹ is —N(R^{A})₂, wherein each R^{A} is independently hydrogen, substituted alkyl, or substituted or unsubstituted heteroalkyl; R² is unsubstituted C₁₋₄ alkyl; and R³ is hydrogen. In certain embodiments, R¹ is —NH₂,

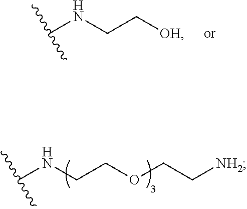

R² is unsubstituted C₁₋₄ alkyl; and R³ is hydrogen. In certain embodiments, R¹ is —Cl, —NH₂,

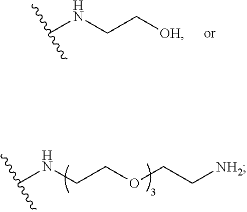

R² is unsubstituted C₁₋₄ alkyl; and R³ is hydrogen. In certain embodiments, R¹ is —Cl; R² is unsubstituted C₁₋₄ alkyl; and R³ is hydrogen. In certain embodiments, R¹ is

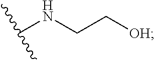 [SMILES: *NCCO]

R² is unsubstituted C₁₋₄ alkyl; and R³ is hydrogen. In certain embodiments, R¹ is

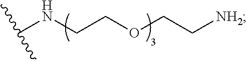

R² is unsubstituted C₁₋₄ alkyl; and R³ is hydrogen.

In certain embodiments, R¹ is —N(R^{A})₂ or halogen; R² is unsubstituted methyl; and R³ is hydrogen. In certain embodiments, R¹ is —N(R^{A})₂, wherein each R^{A} is independently hydrogen, substituted or unsubstituted alkyl, or substituted or unsubstituted heteroalkyl; R² is unsubstituted methyl; and R³ is hydrogen. In certain embodiments, R¹ is —N(R^{A})₂, wherein each R^{A} is independently hydrogen, substituted alkyl, or substituted or unsubstituted heteroalkyl; R² is unsubstituted methyl; and R³ is hydrogen. In certain embodiments, R¹ is —NH₂,

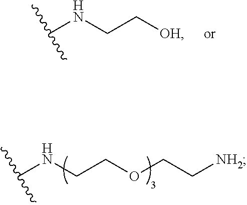

R² is unsubstituted methyl; and R³ is hydrogen. In certain embodiments, R¹ is —Cl, —NH₂,

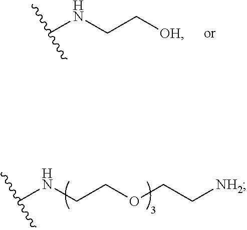

R² is unsubstituted methyl; and R³ is hydrogen. In certain embodiments, R¹ is —Cl; R² is unsubstituted methyl; and R³ is hydrogen. In certain embodiments, R¹ is

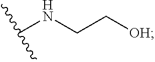 [SMILES: *NCCO]

R² is unsubstituted C₁₋₄ alkyl; and R³ is hydrogen. In certain embodiments, R¹ is

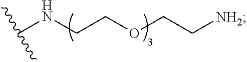

R² is unsubstituted methyl; and R³ is hydrogen.

### R⁴

As described herein, R⁴ is hydrogen, substituted or unsubstituted alkyl, —C(═O)OR^{A}, —C(═O)R^{A}, —C(═O)N(R^{A})₂, or a nitrogen protecting group. In certain embodiments, R⁴ is hydrogen, —C(═O)OR^{A}, —C(═O)R^{A}, —C(═O)N(R^{A})₂, or a nitrogen protecting group. In certain embodiments, R⁴ is hydrogen, —C(═O)R^{A}, or —C(═O)N(R^{A})₂. In certain embodiments, R⁴ is hydrogen, —C(═O)R^{A}, or —C(═O)NHR^{A}. In certain embodiments, R⁴ is hydrogen or —C(═O)N(R^{A})₂. In certain embodiments, R⁴ is hydrogen or —C(═O)NHR^{A}. In certain embodiments, R⁴ is hydrogen or —C(═O)R^{A}. In certain embodiments, R⁴ is —C(═O)R^{A} or —C(═O)N(R^{A})₂. In certain embodiments, R⁴ is —C(═O)R^{A} or —C(═O)NHR^{A}. In certain embodiments, R⁴ is hydrogen.

In certain embodiments, R⁴ is —C(═O)R^{A}. In certain embodiments, R⁴ is —C(═O)R^{A}, wherein R^{A} is unsubstituted alkyl. In certain embodiments, R⁴ is —C(═O)R^{A}, wherein R^{A} is unsubstituted C₁₋₄ alkyl. In certain embodiments, R⁴ is —C(═O)R^{A}, wherein R^{A} is unsubstituted C₁₋₃ alkyl. In certain embodiments, R⁴ is —C(═O)R^{A}, wherein R^{A} is unsubstituted C₁₋₂ alkyl. In certain embodiments, R⁴ is —C(═O)R^{A}, wherein R^{A} is unsubstituted methyl.

In certain embodiments, R⁴ is —C(═O)OR^{A}. In certain embodiments, R⁴ is —C(═O)OR^{A}, wherein R^{A} is unsubstituted alkyl. In certain embodiments, R⁴ is —C(═O)OR^{A}, wherein R^{A} is unsubstituted C₁₋₄ alkyl. In certain embodiments, R⁴ is —C(═O)OR^{A}, wherein R^{A} is tert-butyl.

In certain embodiments, R⁴ is —C(═O)R^{A}, wherein R^{A} is substituted heteroalkyl or substituted alkyl. In certain embodiments, R⁴ is —C(═O)R^{A}, and each R^{A} is independently substituted heteroalkyl or substituted alkyl, wherein the substituted heteroalkyl and alkyl comprise at least one repeat unit of formula

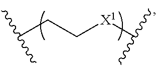

wherein X¹ is —O— or —CH₂—. In certain embodiments, R⁴ is

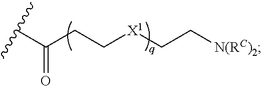

wherein: X¹ is —O— or —CH₂—; q is 1, 2, 3, or 4; and each R^{C} is independently hydrogen, substituted or unsubstituted alkyl, substituted or unsubstituted heteroalkyl, substituted or unsubstituted acyl, substituted or unsubstituted aryl, substituted or unsubstituted heteroaryl, or a nitrogen protecting group. In certain embodiments, R⁴ is

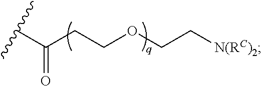

wherein: q is 1, 2, 3, or 4; and each R^{C} is independently hydrogen, substituted or unsubstituted alkyl, substituted or unsubstituted heteroalkyl, substituted or unsubstituted acyl, substituted or unsubstituted aryl, substituted or unsubstituted heteroaryl, or a nitrogen protecting group. In certain embodiments, R⁴ is

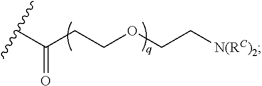

wherein: q is 3; and each R^{C} is independently hydrogen, substituted or unsubstituted alkyl, substituted or unsubstituted heteroalkyl, substituted or unsubstituted acyl, substituted or unsubstituted aryl, substituted or unsubstituted heteroaryl,
or a nitrogen protecting group. In certain embodiments, R⁴ is

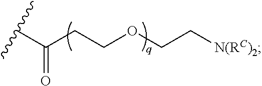

wherein: q is 3; and each R^{C} is independently hydrogen, or substituted or unsubstituted alkyl, or a nitrogen protecting group. In certain embodiments, R⁴ is

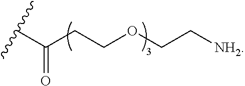

In certain embodiments, R⁴ is

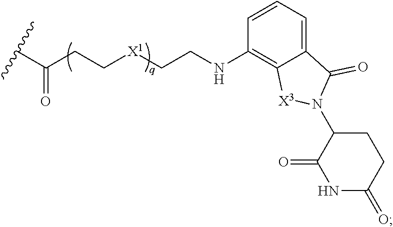

wherein: X¹ is —O— or —CH₂—; q is 1, 2, 3, or 4; and X³ is C═O or CH₂. In certain embodiments, R⁴ is

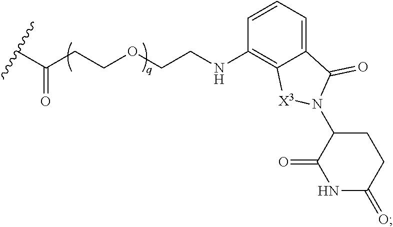

wherein: q is 1, 2, 3, or 4; and X³ is C═O or CH₂. In certain embodiments, R⁴ is

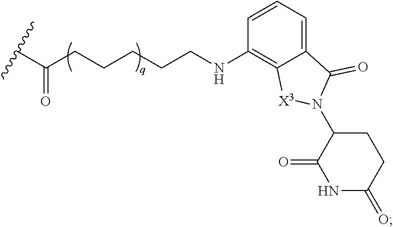

wherein: q is 1, 2, 3, or 4;
and X³ is C═O or CH₂.

In certain embodiments, R⁴ is —C(═O)N(R^{A})₂. In certain embodiments, R⁴ is —C(═O)NHR^{A}. In certain embodiments, R⁴ is —C(═O)NHR^{A}, wherein R^{A} is unsubstituted alkyl. In certain embodiments, R⁴ is —C(═O)NHR^{A}, wherein R^{A} is unsubstituted C₁₋₄ alkyl. In certain embodiments, R⁴ is —C(═O)NHR^{A}, wherein R^{A} is unsubstituted C₁₋₃ alkyl. In certain embodiments, R⁴ is —C(═O)NHR^{A}, wherein R^{A} is unsubstituted C₁₋₂ alkyl. In certain embodiments, R⁴ is —C(═O)NHR^{A}, wherein R^{A} is unsubstituted isopropyl or unsubstituted n-propyl. In certain embodiments, R⁴ is —C(═O)NHR^{A}, wherein R^{A} is unsubstituted isopropyl. In certain embodiments, R⁴ is —C(═O)NHR^{A}, wherein R^{A} is unsubstituted n-propyl.

In certain embodiments, R⁴ is hydrogen or substituted or unsubstituted alkyl. In certain embodiments, R⁴ is hydrogen or substituted or unsubstituted C₁₋₄ alkyl. In certain embodiments, R⁴ is hydrogen.

In certain embodiments, R⁴ is substituted alkyl.

In certain embodiments, R⁴ is alkyl substituted with a substituted or unsubstituted heteroalkyl and a moiety capable of binding an E3 ligase and promoting protein degradation (e.g., degrading MYB). In certain embodiments, R⁴ is alkyl substituted with a a substituted or unsubstituted heteroalkyl and a moiety capable of binding cereblon and promoting protein degradation (e.g., degrading MYB). In certain embodiments, R⁴ is

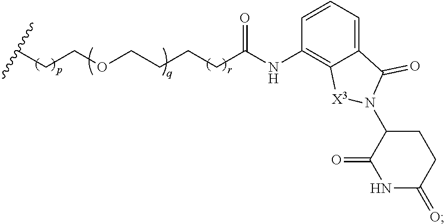

wherein: p is 1-10; q is 1-4; r is 0-6; and X³ is C═O or CH₂. In certain embodiments, R⁴ is

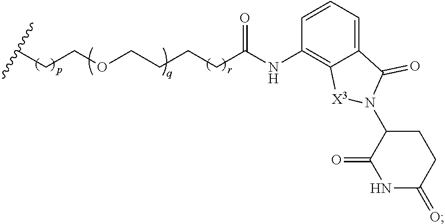

wherein: p is 1-5; q is 1-4; r is 0-3; and X³ is C═O or CH₂. In certain embodiments, R⁴ is

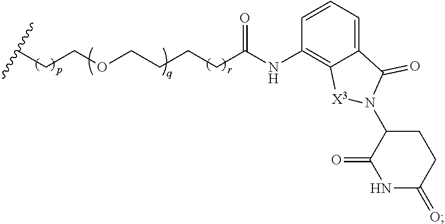

wherein: p is 1-5; q is 3; r is 0-3; and X³ is C═O or CH₂. In certain embodiments, R⁴ is

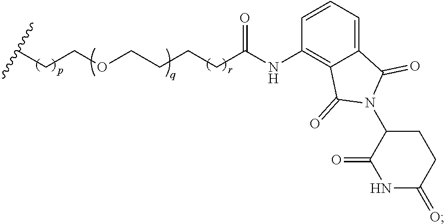

wherein: p is 1-5; q is 3; and r is 0-3. In certain embodiments, R⁴ is

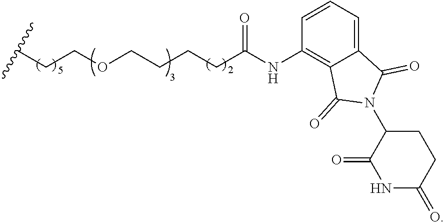

In certain embodiments, R⁴ is

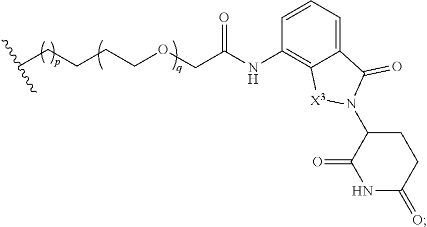

wherein: p is 1-10; q is 1-4; and X³ is C═O or CH₂. In certain embodiments, R⁴ is

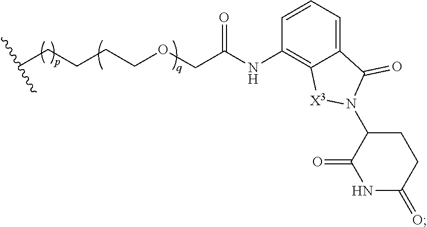

wherein: p is 1-4; q is 1-4; and X³ is C═O or CH₂. In certain embodiments, R⁴ is

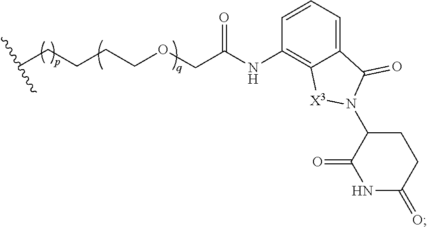

wherein: p is 3; q is 1-4; and X³ is C═O or CH₂. In certain embodiments, R⁴ is

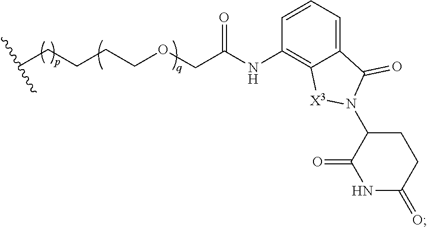

wherein: p is 3; q is 2; and X³ is C═O or CH₂. In certain embodiments, R⁴ is

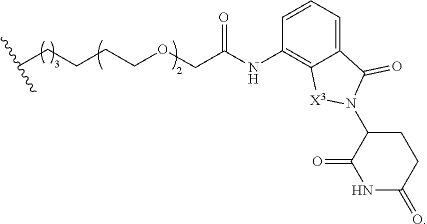

In certain embodiments, R⁴ is substituted alkyl comprising at least one instance of formula

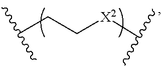

wherein X² is heterocyclyl. In certain embodiments, R⁴ is of formula

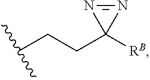

wherein R^{B} is substituted alkyl. In certain embodiments, R⁴ is of formula

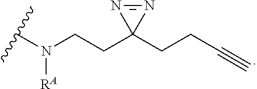

In certain embodiments, R⁴ is of formula

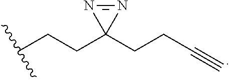 [SMILES: *CCC1(CCC#C)N=N1]

### R⁵

As described herein, each R⁵ is independently —OR^{A}, —N(R^{A})₂, substituted or unsubstituted alkyl, substituted or unsubstituted carbocyclyl, substituted or unsubstituted aryl, substituted or unsubstituted heterocyclyl, or substituted or unsubstituted heteroaryl. In certain embodiments, R⁵ is —OR^{A}, —N(R^{A})₂, substituted or unsubstituted alkyl, substituted or unsubstituted aryl, substituted or unsubstituted heterocyclyl, or substituted or unsubstituted heteroaryl. In certain embodiments, R⁵ is —OR^{A}, —N(R^{A})₂, substituted or unsubstituted heterocyclyl, or substituted or unsubstituted heteroaryl. In certain embodiments, R⁵ is —OR^{A}, —N(R^{A})₂, or substituted or unsubstituted heteroaryl. In certain embodiments, R⁵ is —OR^{A}, —N(R^{A})₂, or substituted or unsubstituted heteroaryl; and s is 1. In certain embodiments, R⁵ is —OR^{A}, —N(R^{A})₂, or substituted or unsubstituted heterocyclyl; and s is 1.

In certain embodiments, R⁵ is 5- to 6-membered heterocyclyl or 5- to 6-membered heteroaryl. In certain embodiments, R⁵ is 5- to 6-membered heterocyclyl. In certain embodiments, R⁵ is 5- to 6-membered heteroaryl. In certain embodiments, R⁵ is morpholinyl, piperazinyl, piperidinyl, pyrrolidinyl, pyrrolyl, pyrazolyl, pyrazolinyl, triazolyl, tetrazolyl, or imidazolyl. In certain embodiments, R⁵ is piperidinyl, pyrrolidinyl, pyrrolyl, pyrazolyl, pyrazolinyl, triazolyl, tetrazolyl, or imidazolyl. In certain embodiments, R⁵ is morpholinyl, piperazinyl, piperidinyl, pyrrolidinyl, or pyrazolinyl. In certain embodiments, R⁵ is piperidinyl, pyrrolidinyl, or pyrazolinyl. In certain embodiments, R⁵ is pyrrolyl, pyrazolyl, triazolyl, tetrazolyl, or imidazolyl. In certain embodiments, R⁵ is morpholinyl. In certain embodiments, R⁵ is piperazinyl. In certain embodiments, R⁵ is N-methylpiperazinyl. In certain embodiments, R⁵ is pyrrolidinyl. In certain embodiments, R⁵ is imidazolyl. In certain embodiments, R⁵ is pyrazolyl. In certain embodiments, R⁵ is piperidinyl. In certain embodiments, R⁵ is pyrrolyl. In certain embodiments, R⁵ is pyrazolinyl. In certain embodiments, R⁵ is triazolyl. In certain embodiments, R⁵ is tetrazolyl.

In certain embodiments, R⁵ is —OR^{A}. In certain embodiments, R⁵ is —OR^{A}, wherein R^{A} is substituted or unsubstituted alkyl. In certain embodiments, R⁵ is —OR^{A}, wherein R^{A} is substituted or unsubstituted alkyl. In certain embodiments, R⁵ is —OR^{A}, wherein R^{A} is substituted or unsubstituted C₁₋₄ alkyl. In certain embodiments, R⁵ is —OR^{A}, wherein R^{A} is substituted or unsubstituted C₁₋₃ alkyl. In certain embodiments, R⁵ is —OR^{A}, wherein R^{A} is substituted or unsubstituted C₁₋₂ alkyl. In certain embodiments, R⁵ is —OCH₃.

In certain embodiments, R⁵ is —N(R^{A})₂. In certain embodiments, R⁵ is —N(R^{A})₂, wherein R^{A} is substituted or unsubstituted alkyl. In certain embodiments, R⁵ is —N(R^{A})₂, wherein R^{A} is substituted or unsubstituted alkyl. In certain embodiments, R⁵ is —N(R^{A})₂, wherein R^{A} is substituted or unsubstituted C₁₋₄ alkyl. In certain embodiments, R⁵ is —N(R^{A})₂, wherein R^{A} is substituted or unsubstituted C₁₋₃ alkyl. In certain embodiments, R⁵ is —N(R^{A})₂, wherein R^{A} is substituted or unsubstituted C₁₋₂ alkyl. In certain embodiments, R⁵ is —N(CH₃)₂ or —N(Et)₂. In certain embodiments, R⁵ is —N(Et)₂. In certain embodiments, R⁵ is —N(CH₃)₂.

In certain embodiments, R⁵ is substituted or unsubstituted alkyl. In certain embodiments, R⁵ is substituted or unsubstituted C₁₋₆ alkyl. In certain embodiments, R⁵ is unsubstituted C₁₋₆ alkyl. In certain embodiments, R⁵ is t-butyl.

In certain embodiments, s is 0, 1, 2, 3, 4, or 5. In certain embodiments, s is 0, 1, 2, 3, or 4. In certain embodiments, s is 0, 1, 2, or 3. In certain embodiments, s is 0, 1, or 2. In certain embodiments, s is 0 or 1. In certain embodiments, s is 0. In certain embodiments, s is 1. In certain embodiments, s is 2. In certain embodiments, s is 3. In certain embodiments, s is 4. In certain embodiments, s is 5.

R⁶ and R⁷

As described herein, R⁶ and R⁷ are each independently hydrogen, halogen, or substituted or unsubstituted alkyl, or R⁶ and R⁷ together with the carbon to which they are attached form a carbonyl. In certain embodiments, R⁶ and R⁷ are each independently hydrogen or halogen, or R⁶ and R⁷ together with the carbon to which they are attached form a carbonyl. In certain embodiments, R⁶ and R⁷ are each hydrogen, or R⁶ and R⁷ together with the carbon to which they are attached form a carbonyl. In certain embodiments, R⁶ and R⁷ are each hydrogen. In certain embodiments, R⁶ and R⁷ together with the carbon to which they are attached form a carbonyl.

### T

As described herein, T is

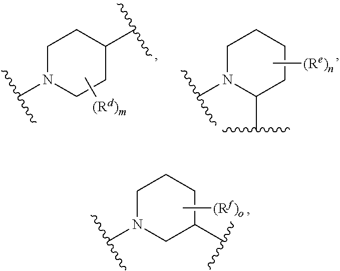

or —NR^{m}—(CR^{g}R^{h})₃—.

As described herein, T is

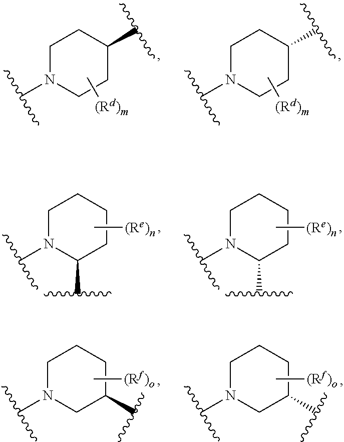

or —NR^{m}—(CR^{g}R^{h})₃—.

As described herein, T is

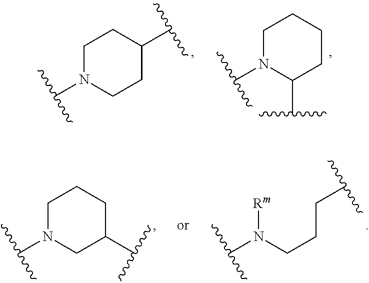

or —NR^{m}—(CR^{g}R^{h})₃—.

As described herein, T is

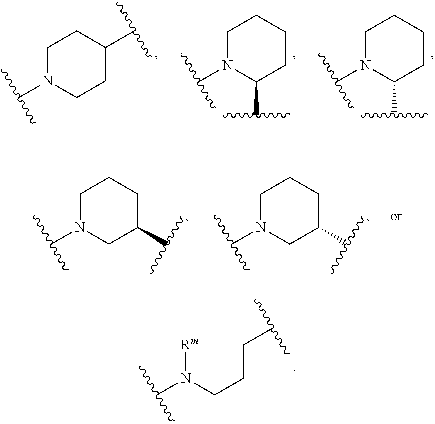

or —NR^{m}—(CR^{g}R^{h})₃—.

As described herein, T is

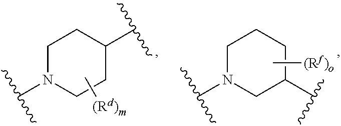

or —NR^{m}—(CR^{g}R^{h})₃—.

As described herein, T is

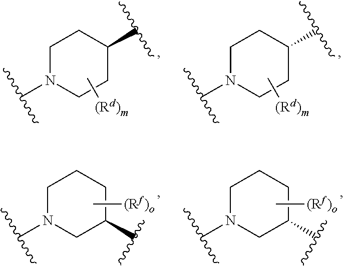

or —NR^{m}—(CR^{g}R^{h})₃—.

In certain embodiments, T is:

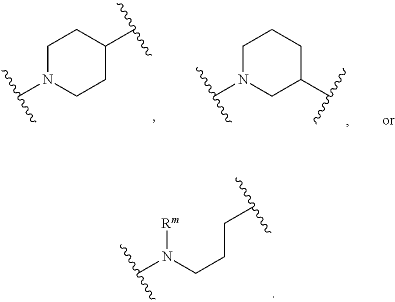

In certain embodiments, T is:

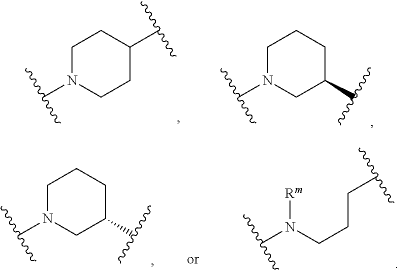

In certain embodiments, T is

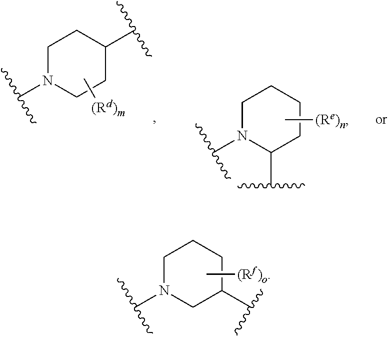

In certain embodiments, T is

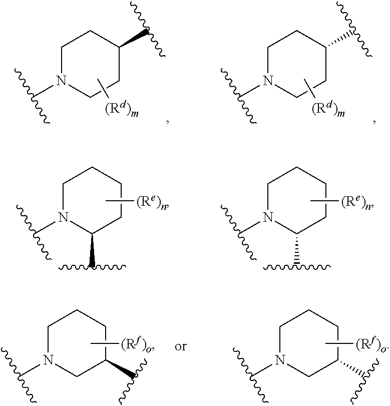

In certain embodiments, T is:

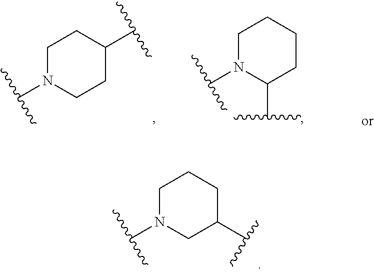 [SMILES: *C1CCN(*)CC1 ; *C1CCCCN1* ; *C1CCCN(*)C1]

In certain embodiments, T is:

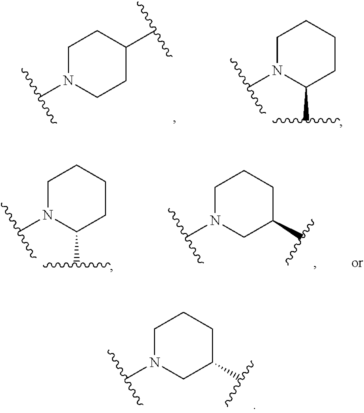 [SMILES: *C1CCN(*)CC1 ; *[C@@H]1CCCCN1* ; *[C@H]1CCCCN1* ; *[C@@H]1CCCN(*)C1 ; *[C@H]1CCCN(*)C1]

In certain embodiments, T is

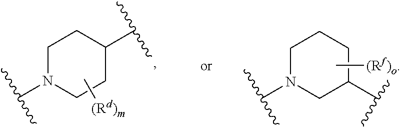

In certain embodiments, T is

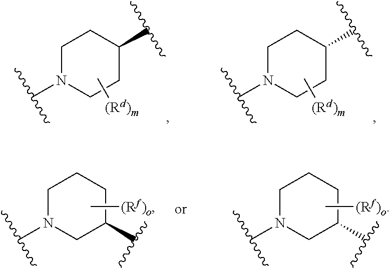

In certain embodiments, T is:

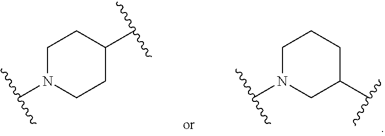 [SMILES: *C1CCN(*)CC1 ; *C1CCCN(*)C1]

In certain embodiments, T is:

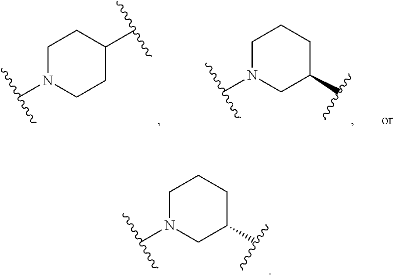 [SMILES: *C1CCN(*)CC1 ; *[C@@H]1CCCN(*)C1 ; *[C@H]1CCCN(*)C1]

In certain embodiments, T is:

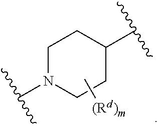

In certain embodiments, T is:

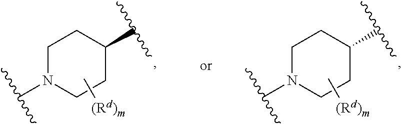

In certain embodiments, T is:

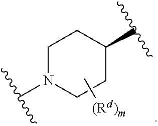

In certain embodiments, T is:

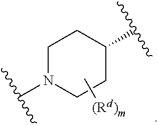

In certain embodiments, T is:

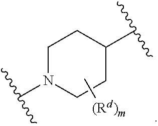

In certain embodiments, T is:

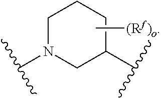

In certain embodiments, T is:

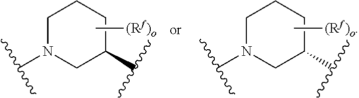

In certain embodiments, T is:

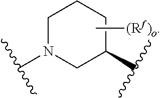

In certain embodiments, T is:

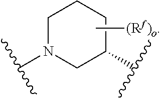

In certain embodiments, T is:

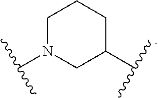 [SMILES: *C1CCCN(*)C1]

In certain embodiments, T is:

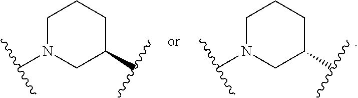 [SMILES: *[C@@H]1CCCN(*)C1 ; *[C@H]1CCCN(*)C1]

In certain embodiments, T is:

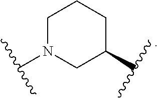 [SMILES: *[C@@H]1CCCN(*)C1]

In certain embodiments, T is:

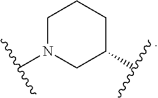 [SMILES: *[C@H]1CCCN(*)C1]

In certain embodiments, T is:

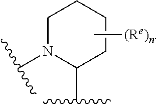

In certain embodiments, T is

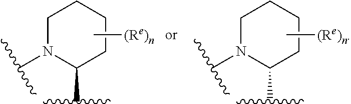

In certain embodiments, T is:

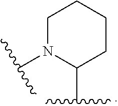 [SMILES: *C1CCCCN1*]

In certain embodiments, T is:

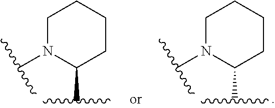 [SMILES: *[C@@H]1CCCCN1* ; *[C@H]1CCCCN1*]

In certain embodiments, T is —NR^{m}—(CR^{g}R^{h})₃—.

In certain embodiments, T is:

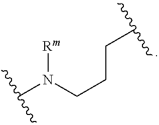

### R^{m}

As described herein, R^{m} is hydrogen, substituted or unsubstituted alkyl, —C(═O)OR^{A}, —C(═O)R^{A}, —C(═O)N(R^{A})₂, or a nitrogen protecting group. In certain embodiments, R^{m} is hydrogen, —C(═O)OR^{A}, —C(═O)R^{A}, —C(═O)N(R^{A})₂, or a nitrogen protecting group. In certain embodiments, R^{m} is hydrogen, —C(═O)R^{A}, or —C(═O)N(R^{A})₂. In certain embodiments, R^{m} is hydrogen, —C(═O)R^{A}, or —C(═O)NHR^{A}. In certain embodiments, R^{m} is hydrogen or —C(═O)N(R^{A})₂. In certain embodiments, R^{m} is hydrogen or —C(═O)NHR^{A}. In certain embodiments, R^{m} is hydrogen or —C(═O)R^{A}. In certain embodiments, R⁴ is —C(═O)R^{A} or —C(═O)N(R^{A})₂. In certain embodiments, R^{m} is —C(═O)R^{A} or —C(═O)NHR^{A}.

In certain embodiments, R^{m} is —C(═O)R^{A}. In certain embodiments, R⁴ is —C(═O)R^{A}, wherein R^{A} is unsubstituted alkyl. In certain embodiments, R^{m} is —C(═O)R^{A}, wherein R^{A} is unsubstituted C₁₋₄ alkyl. In certain embodiments, R^{m} is —C(═O)R^{A}, wherein R^{A} is unsubstituted C₁₋₃ alkyl. In certain embodiments, R^{m} is —C(═O)R^{A}, wherein R^{A} is unsubstituted C₁₋₂ alkyl.

In certain embodiments, R^{m} is —C(═O)N(R^{A})₂. In certain embodiments, R^{m} is —C(═O)NHR^{A}. In certain embodiments, R^{m} is —C(═O)NHR^{A}, wherein R^{A} is unsubstituted alkyl. In certain embodiments, R^{m} is —C(═O)NHR^{A}, wherein R^{A} is unsubstituted C₁₋₄ alkyl. In certain embodiments, R^{m} is —C(═O)NHR^{A}, wherein R^{A} is unsubstituted C₁₋₃ alkyl. In certain embodiments, R^{m} is —C(═O)NHR^{A}, wherein R^{A} is unsubstituted C₁₋₂ alkyl.

In certain embodiments, R^{m} is hydrogen or substituted or unsubstituted alkyl. In certain embodiments, R^{m} is hydrogen or substituted or unsubstituted C₁₋₄ alkyl. In certain embodiments, R^{m} is hydrogen.

R^{d}, R^{e}, R^{f}, R^{g}, and R^{h}

As described herein, R^{d}, R^{e}, R^{f}, R^{g}, and R^{h} are each independently halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A}.

In certain embodiments, R^{d}, R^{e}, R^{f}, R^{g}, and R^{h} are each independently halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroalkyl. In certain embodiments, R^{d}, R^{e}, R^{f}, R^{g}, and R^{h} are each independently halogen, cyano, substituted or unsubstituted alkyl, substituted or unsubstituted alkenyl, substituted or unsubstituted alkynyl, substituted or unsubstituted carbocyclyl, or substituted or unsubstituted heterocyclyl. In certain embodiments, R^{d}, R^{e}, R^{f}, R^{g}, and R^{h} are each independently halogen, cyano, substituted or unsubstituted alkyl, substituted or unsubstituted alkenyl, or substituted or unsubstituted alkynyl. In certain embodiments, R^{d}, R^{e}, R^{f}, R^{g}, and R^{h} are each independently halogen, cyano, or substituted or unsubstituted alkyl. In certain embodiments, R^{d}, R^{e}, R^{f}, R^{g}, and R^{h} are each independently halogen, or substituted or unsubstituted alkyl. In certain embodiments, R^{d}, R^{e}, R^{f}, R^{g}, and R^{h} are each independently halogen. In certain embodiments, R^{d}, R^{e}, R^{f}, R^{g}, and R^{h} are each independently substituted or unsubstituted alkyl. In certain embodiments, R^{d}, R^{e}, R^{f}, R⁹, and R^{h} are each independently unsubstituted alkyl.

In certain embodiments, m is 0, 1, 2, 3, 4, or 5. In certain embodiments, m is 0, 1, 2, 3, or 4. In certain embodiments, m is 0, 1, 2, or 3. In certain embodiments, m is 0, 1, or 2. In certain embodiments, m is 0 or 1. In certain embodiments, m is 0. In certain embodiments, m is 1.

In certain embodiments, n is 0, 1, 2, 3, 4, or 5. In certain embodiments, n is 0, 1, 2, 3, or 4. In certain embodiments, n is 0, 1, 2, or 3. In certain embodiments, n is 0, 1, or 2. In certain embodiments, n is 0 or 1. In certain embodiments, n is 0. In certain embodiments, n is 1.

In certain embodiments, o is 0, 1, 2, 3, 4, or 5. In certain embodiments, o is 0, 1, 2, 3, or 4. In certain embodiments, o is 0, 1, 2, or 3. In certain embodiments, o is 0, 1, or 2. In certain embodiments, o is 0 or 1. In certain embodiments, o is 0. In certain embodiments, o is 1.

### R^{A}

As described herein, each occurrence of R^{A} is, independently, hydrogen, substituted or unsubstituted alkyl, substituted or unsubstituted heteroalkyl, substituted or unsubstituted acyl, an oxygen protecting group when bound to an oxygen, or a nitrogen protecting group when bound to a nitrogen, or two R^{A} groups are joined to form a substituted or unsubstituted heterocyclic ring or substituted or unsubstituted heteroaryl ring.

In certain embodiments, each occurrence of R^{A} is, independently, hydrogen, substituted or unsubstituted C₁₋₆ alkyl, substituted or unsubstituted heteroalkyl, or two R^{A} groups are joined to form a substituted or unsubstituted heterocyclic ring.

In certain embodiments, each occurrence of R^{A} is, independently, hydrogen, substituted or unsubstituted C₁₋₆ alkyl, substituted or unsubstituted C₁₋₃₀ heteroalkyl, or two R^{A} groups are joined to form a substituted or unsubstituted heterocyclic ring.

In certain embodiments, each occurrence of R^{A} is, independently, hydrogen, substituted or unsubstituted C₁₋₆ alkyl, or substituted or unsubstituted C₁₋₃₀ heteroalkyl. In certain embodiments, each occurrence of R^{A} is, independently, hydrogen, substituted or unsubstituted C₁₋₆ alkyl, or substituted or unsubstituted C₁₋₂₀ heteroalkyl. In certain embodiments, each occurrence of R^{A} is, independently, hydrogen, or substituted or unsubstituted C₁₋₆ alkyl. In certain embodiments, each occurrence of R^{A} is hydrogen.

### Further Embodiments of Formula (I)

In certain embodiments, the compound of Formula (I) is a compound of Formula (I-a):

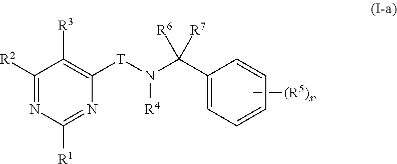

or a pharmaceutically acceptable salt, co-crystal, tautomer, stereoisomer, solvate, hydrate, polymorph, isotopically enriched derivative, or prodrug thereof, wherein T, R¹, R², R³, R⁴, R⁵, R⁶, R⁷, and s are as defined herein.

In certain embodiments, the compound of Formula (I) is a compound of Formula (I-b):

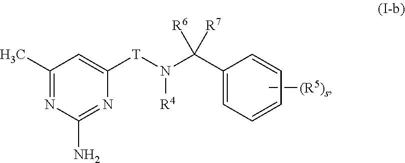

or a pharmaceutically acceptable salt, co-crystal, tautomer, stereoisomer, solvate, hydrate, polymorph, isotopically enriched derivative, or prodrug thereof, wherein T, R⁴, R⁵, R⁶, R⁷, and s are as defined herein.

In certain embodiments, the compound of Formula (I) is a compound of Formula (I-c):

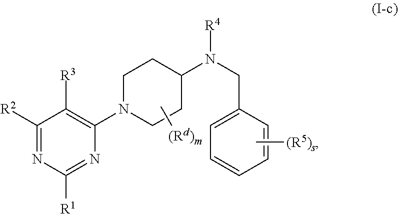

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R^{d}, m, and s are as defined herein.

In certain embodiments, the compound of Formula (I) is a compound of Formula (I-c-1):

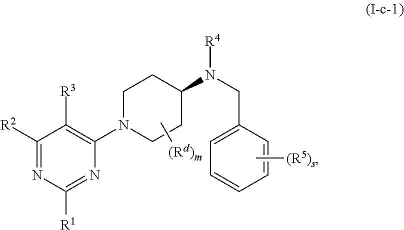

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R^{d}, m, and s are as defined herein; and m is at least 1.

In certain embodiments, the compound of Formula (I) is a compound of Formula (I-b-2):

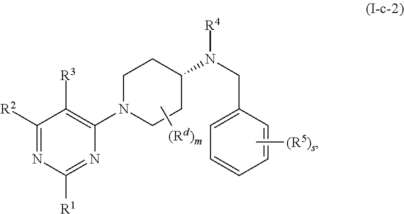

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R^{d}, m, and s are as defined herein; and m is at least 1.

In certain embodiments, the compound of Formula (I) is a compound of Formula (I-c-3):

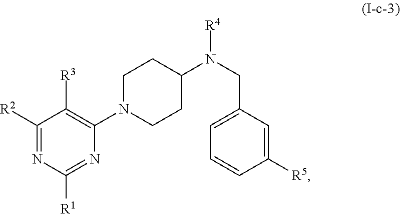

or a pharmaceutically acceptable salt, co-crystal, tautomer, stereoisomer, solvate, hydrate, polymorph, isotopically enriched derivative, or prodrug thereof, wherein R¹, R², R³, R⁴, and R⁵ are as defined herein.

In certain embodiments, the compound of Formula (I) is a compound of Formula (I-c-4):

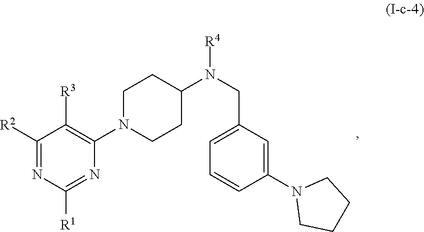

or a pharmaceutically acceptable salt, co-crystal, tautomer, stereoisomer, solvate, hydrate, polymorph, isotopically enriched derivative, or prodrug thereof, wherein R¹, R², R³, R⁴, and R⁵ are as defined herein.

In certain embodiments, the compound of Formula (I) is a compound of Formula (I-d):

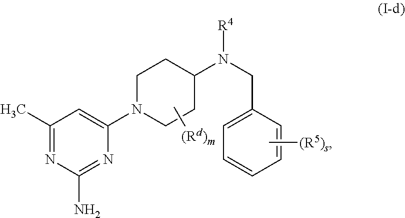

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-d-1):

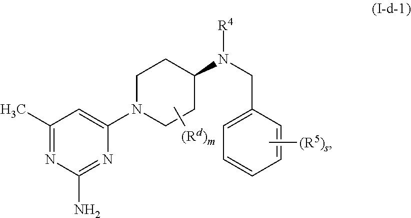

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein; and m is at least 1.

In certain embodiments, the compound of Formula (I) is a compound of Formula (I-d-2):

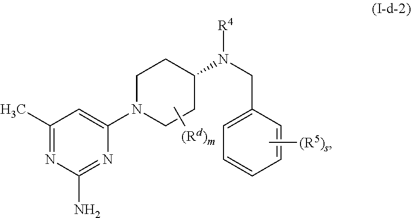

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein; and m is at least 1.

In certain embodiments, the compound of Formula (I) is a compound of Formula (I-e):

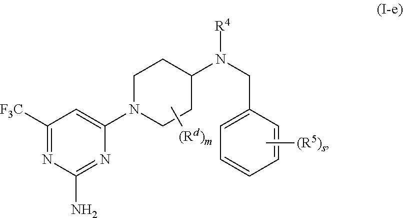

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-f):

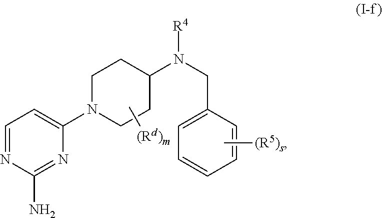

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-g):

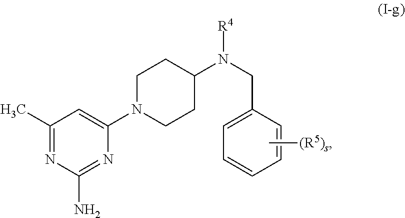

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-g-1):

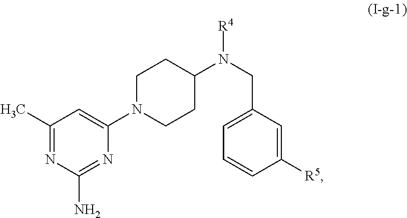

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula (I-g-2):

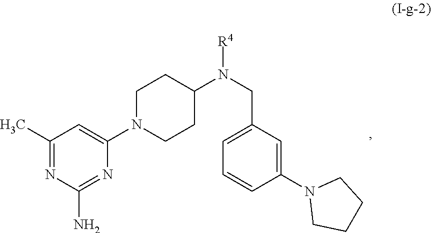

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of Formula (I-g-3):

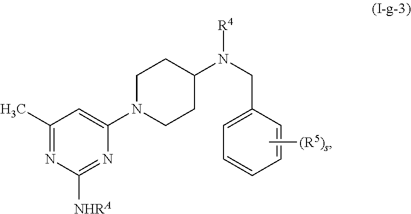

or a pharmaceutically acceptable salt, co-crystal, tautomer, stereoisomer, solvate, hydrate, polymorph, isotopically enriched derivative, or prodrug thereof, wherein R^{A}, R⁴, R⁵, and s are as defined herein.

In certain embodiments, the compound of Formula (I) is a compound of Formula (I-g-4):

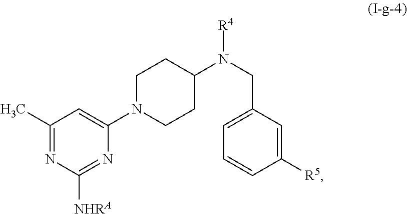

or a pharmaceutically acceptable salt, co-crystal, tautomer, stereoisomer, solvate, hydrate, polymorph, isotopically enriched derivative, or prodrug thereof, wherein R^{A}, R⁴ and R⁵ are as defined herein.

In certain embodiments, the compound of Formula (I) is a compound of Formula (I-g-5):

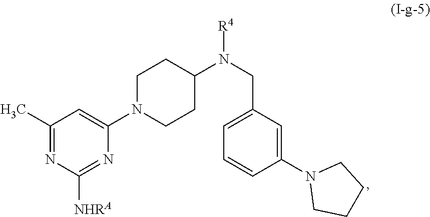

or a pharmaceutically acceptable salt, co-crystal, tautomer, stereoisomer, solvate, hydrate, polymorph, isotopically enriched derivative, or prodrug thereof, wherein R^{A} and R⁴ are as defined herein.

In certain embodiments, the compound of Formula (I) is a compound of Formula (I-g-6):

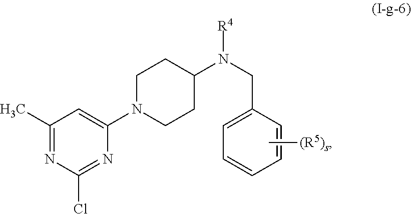

or a pharmaceutically acceptable salt, co-crystal, tautomer, stereoisomer, solvate, hydrate, polymorph, isotopically enriched derivative, or prodrug thereof, wherein R^{A}, R⁴, R⁵, and s are as defined herein.

In certain embodiments, the compound of Formula (I) is a compound of Formula (I-g-7):

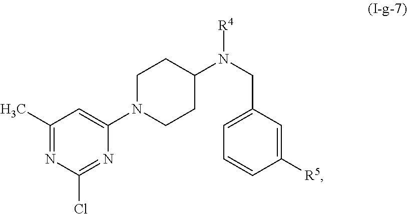

or a pharmaceutically acceptable salt, co-crystal, tautomer, stereoisomer, solvate, hydrate, polymorph, isotopically enriched derivative, or prodrug thereof, wherein R^{A}, R⁴ and R⁵ are as defined herein.

In certain embodiments, the compound of Formula (I) is a compound of Formula (I-g-8):

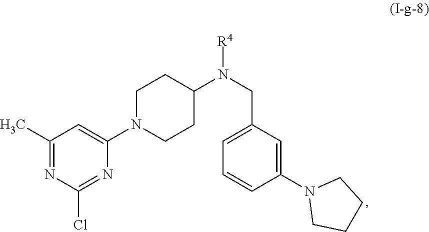

or a pharmaceutically acceptable salt, co-crystal, tautomer, stereoisomer, solvate, hydrate, polymorph, isotopically enriched derivative, or prodrug thereof, wherein R^{A} and R⁴ are as defined herein.

In certain embodiments, the compound of Formula (I) is a compound of Formula (I-g-9):

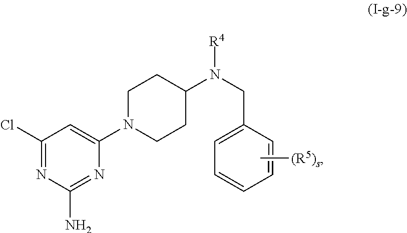

or a pharmaceutically acceptable salt, co-crystal, tautomer, stereoisomer, solvate, hydrate, polymorph, isotopically enriched derivative, or prodrug thereof, wherein R^{A}, R⁴, R⁵, and s are as defined herein.

In certain embodiments, the compound of Formula (I) is a compound of Formula (I-g-10):

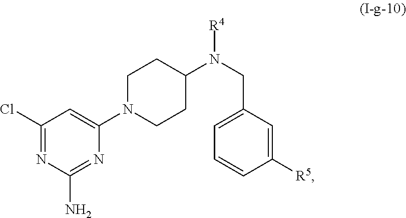

or a pharmaceutically acceptable salt, co-crystal, tautomer, stereoisomer, solvate, hydrate, polymorph, isotopically enriched derivative, or prodrug thereof, wherein R^{A}, R⁴ and R⁵ are as defined herein.

In certain embodiments, the compound of Formula (I) is a compound of Formula (I-g-11):

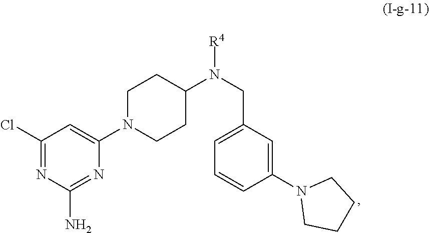

or a pharmaceutically acceptable salt, co-crystal, tautomer, stereoisomer, solvate, hydrate, polymorph, isotopically enriched derivative, or prodrug thereof, wherein R^{A} and R⁴ are as defined herein.

In certain embodiments, the compound of Formula (I) is a compound of Formula (I-g-12):

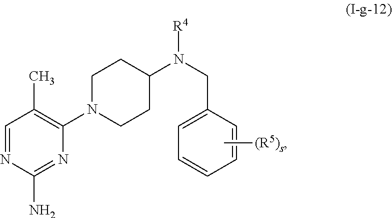

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-g-13):

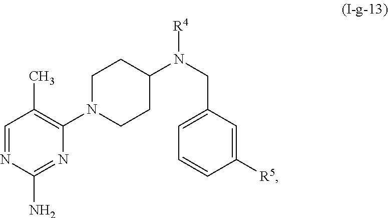

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula (I-g-14):

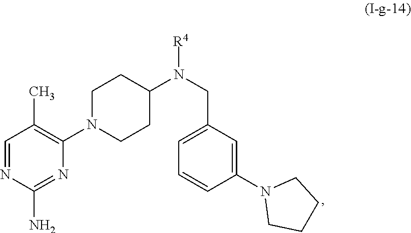

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of Formula (I-h):

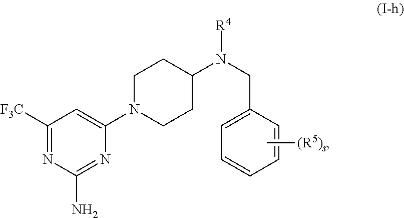

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-h-1):

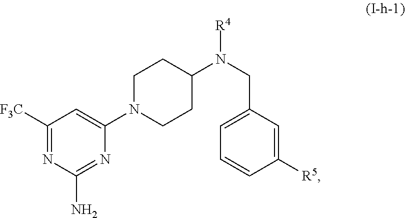

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula (I-h-2):

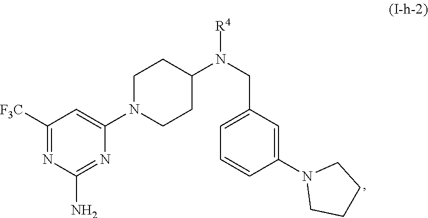

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of Formula (I-i):

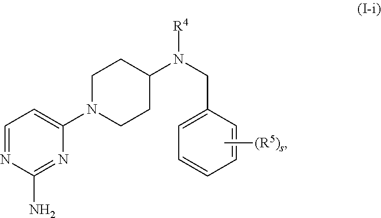

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-j):

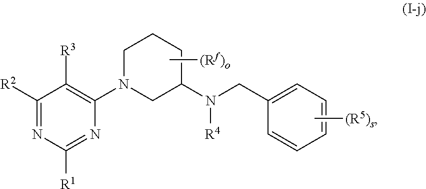

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R^{f}, o, and s are as defined herein.

In certain embodiments, the compound of Formula (I) is a compound of Formula (I-j-1):

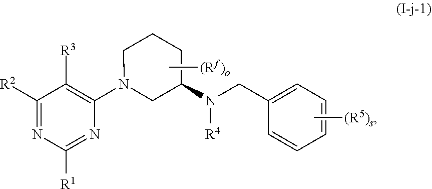

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R^{f}, o, and s are as defined herein.

In certain embodiments, the compound of Formula (I) is a compound of Formula (I-j-2):

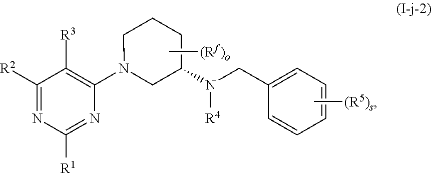

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R^{f}, o, and s are as defined herein.

In certain embodiments, the compound of Formula (I) is a compound of Formula (I-k):

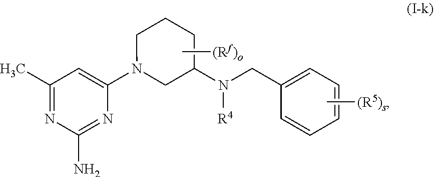

or a pharmaceutically acceptable salt, co-crystal, tautomer, stereoisomer, solvate, hydrate, polymorph, isotopically enriched derivative, or prodrug thereof, wherein R⁴, R⁵, R^{f}, o, and s are as defined herein.

In certain embodiments, the compound of Formula (I) is a compound of Formula (I-k-1):

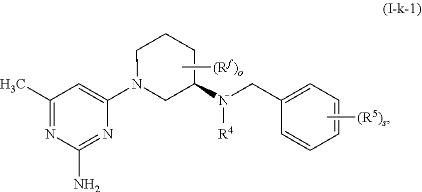

or a pharmaceutically acceptable salt, co-crystal, tautomer, stereoisomer, solvate, hydrate, polymorph, isotopically enriched derivative, or prodrug thereof, wherein R⁴, R⁵, R^{f}, o, and s are as defined herein.

In certain embodiments, the compound of Formula (I) is a compound of Formula (I-k-2):

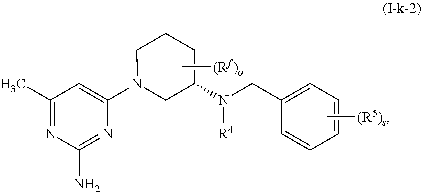

or a pharmaceutically acceptable salt, co-crystal, tautomer, stereoisomer, solvate, hydrate, polymorph, isotopically enriched derivative, or prodrug thereof, wherein R⁴, R⁵, R^{f}, o, and s are as defined herein.

In certain embodiments, the compound of Formula (I) is a compound of Formula (I-i):

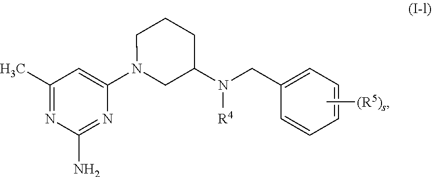

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-l-1):

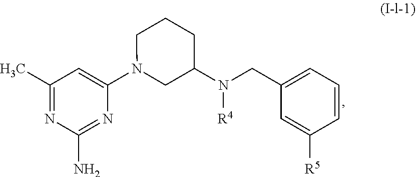

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula (I-l-2):

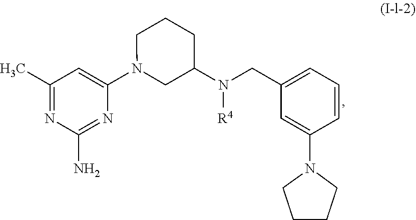

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of Formula (I-m):

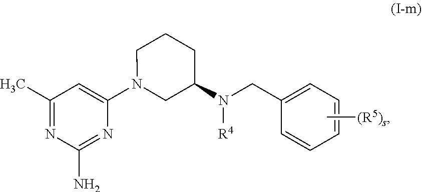

or a pharmaceutically acceptable salt, co-crystal, tautomer, stereoisomer, solvate, hydrate, polymorph, isotopically enriched derivative, or prodrug thereof, wherein R⁵ and s are as defined herein.

In certain embodiments, the compound of Formula (I) is a compound of Formula (I-m-1):

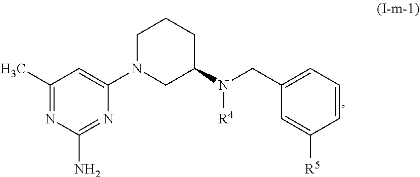

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula (I-m-2):

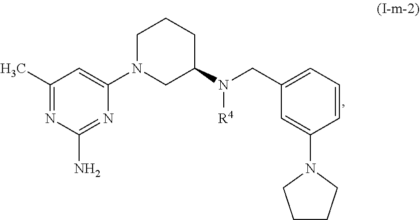

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of Formula (I-n):

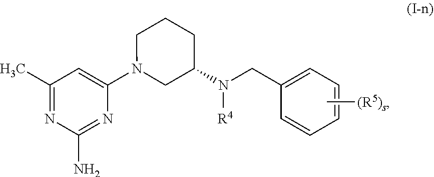

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-o):

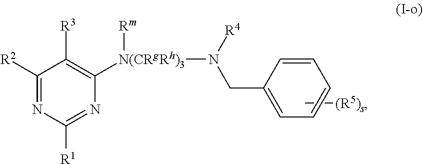

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R^{aa}, R^{g}, R^{h}, and s are as defined herein.

In certain embodiments, the compound of Formula (I) is a compound of Formula (I-p):

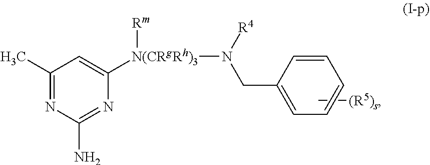

or a pharmaceutically acceptable salt, co-crystal, tautomer, stereoisomer, solvate, hydrate, polymorph, isotopically enriched derivative, or prodrug thereof, wherein R⁴, R⁵, R^{m}, R^{g}, R^{h} and s are as defined herein.

In certain embodiments, the compound of Formula (I) is a compound of Formula (I-q):

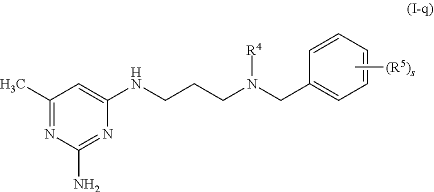

or a pharmaceutically acceptable salt, co-crystal, tautomer, stereoisomer, solvate, hydrate, polymorph, isotopically enriched derivative, or prodrug thereof, wherein R⁴, R⁵ and s are as defined herein.

In certain embodiments, the compound of Formula (I) is a compound of Formula (I-r):

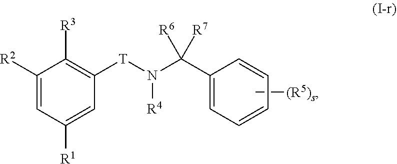

or a pharmaceutically acceptable salt, co-crystal, tautomer, stereoisomer, solvate, hydrate, polymorph, isotopically enriched derivative, or prodrug thereof, wherein T, R¹, R², R³, R⁴, R⁵, R⁶, R⁷, and s are as defined herein.

In certain embodiments, the compound of Formula (I) is a compound of Formula (I-s):

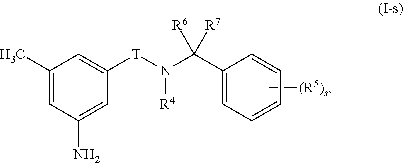

or a pharmaceutically acceptable salt, co-crystal, tautomer, stereoisomer, solvate, hydrate, polymorph, isotopically enriched derivative, or prodrug thereof, wherein T, R⁴, R⁵, R⁶, R⁷, and s are as defined herein.

In certain embodiments, the compound of Formula (I) is a compound of Formula (I-t):

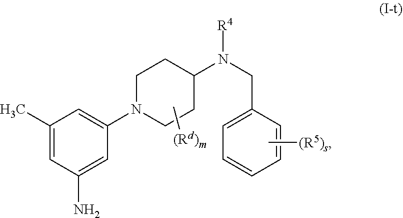

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-u):

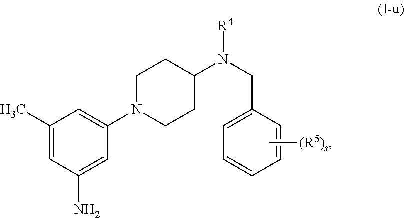

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-v):

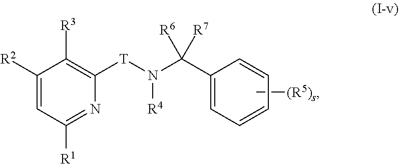

or a pharmaceutically acceptable salt, co-crystal, tautomer, stereoisomer, solvate, hydrate, polymorph, isotopically enriched derivative, or prodrug thereof, wherein T, R¹, R², R³, R⁴, R⁵, R⁶, R⁷, and s are as defined herein.

In certain embodiments, the compound of Formula (I) is a compound of Formula (I-w):

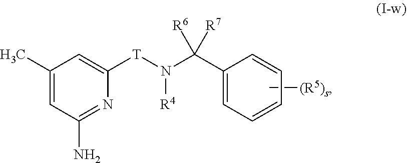

or a pharmaceutically acceptable salt, co-crystal, tautomer, stereoisomer, solvate, hydrate, polymorph, isotopically enriched derivative, or prodrug thereof, wherein T, R⁴, R⁵, R⁶, R⁷, and s are as defined herein.

In certain embodiments, the compound of Formula (I) is a compound of Formula (I-x):

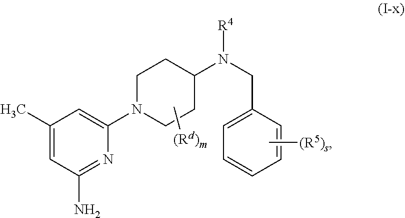

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-y):

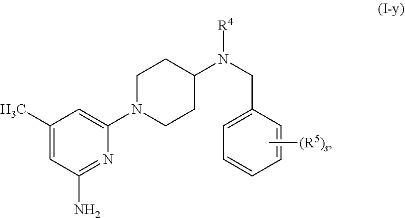

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-y-1):

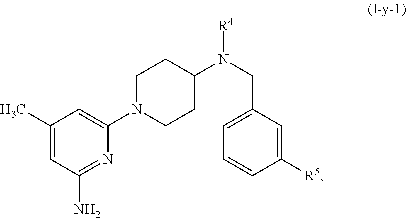

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula (I-y-2):

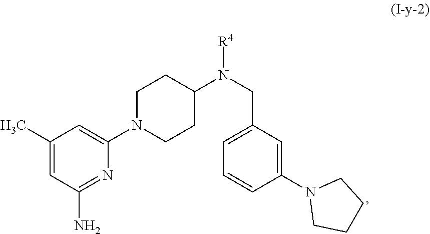

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of Formula (I-z):

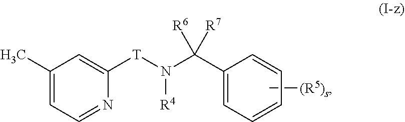

or a pharmaceutically acceptable salt, co-crystal, tautomer, stereoisomer, solvate, hydrate, polymorph, isotopically enriched derivative, or prodrug thereof, wherein T, R⁴, R⁵, R⁶, R⁷, and s are as defined herein.

In certain embodiments, the compound of Formula (I) is a compound of Formula (I-aa):

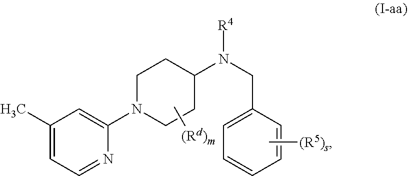

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-bb):

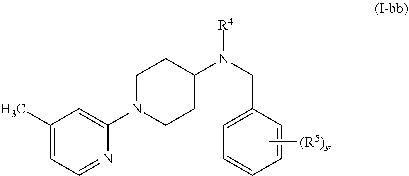

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-bb-1):

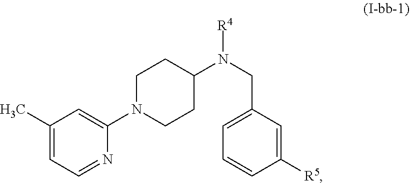

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula

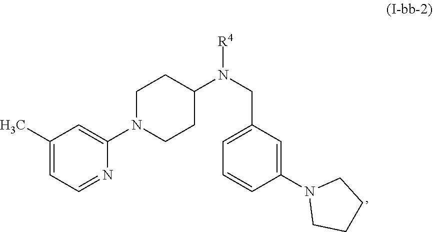

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of Formula (I-cc):

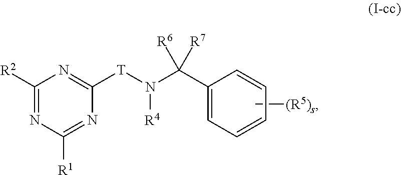

or a pharmaceutically acceptable salt, co-crystal, tautomer, stereoisomer, solvate, hydrate, polymorph, isotopically enriched derivative, or prodrug thereof, wherein T, R¹, R², R⁴, R⁵, R⁶, R⁷, and s are as defined herein.

In certain embodiments, the compound of Formula (I) is a compound of Formula (I-dd):

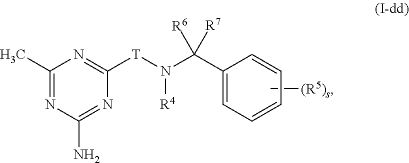

or a pharmaceutically acceptable salt, co-crystal, tautomer, stereoisomer, solvate, hydrate, polymorph, isotopically enriched derivative, or prodrug thereof, wherein T, R⁴, R⁵, R⁶, R⁷, and s are as defined herein.

In certain embodiments, the compound of Formula (I) is a compound of Formula (I-ee):

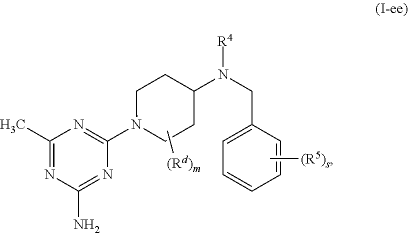

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-ff):

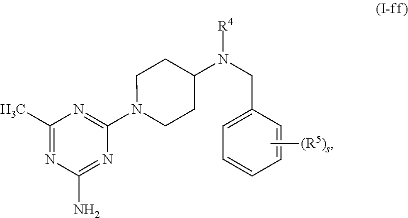

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-gg):

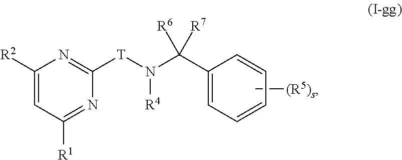

or a pharmaceutically acceptable salt, co-crystal, tautomer, stereoisomer, solvate, hydrate, polymorph, isotopically enriched derivative, or prodrug thereof, wherein T, R¹, R², R⁴, R⁵, R⁶, R⁷, and s are as defined herein.

In certain embodiments, the compound of Formula (I) is a compound of Formula (I-hh):

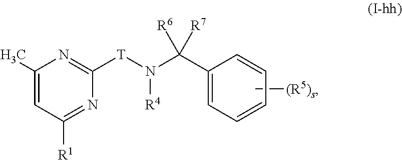

or a pharmaceutically acceptable salt, co-crystal, tautomer, stereoisomer, solvate, hydrate, polymorph, isotopically enriched derivative, or prodrug thereof, wherein R⁴, R⁵, R⁶, R⁷, and s are as defined herein.

In certain embodiments, the compound of Formula (I) is a compound of Formula (I-ii):

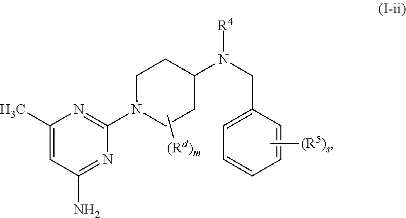

or a pharmaceutically acceptable salt, co-crystal, tautomer, stereoisomer, solvate, hydrate, polymorph, isotopically enriched derivative, or prodrug thereof, wherein R⁴, R⁵, R^{d}, m, and s are as defined herein.

In certain embodiments, the compound of Formula (I) is a compound of Formula (I-jj):

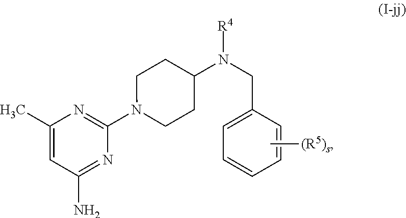

or a pharmaceutically acceptable salt, co-crystal, tautomer, stereoisomer, solvate, hydrate, polymorph, isotopically enriched derivative, or prodrug thereof, wherein R⁴, R⁵, and s are as defined herein.

In certain embodiments, the compound of Formula (I) is a compound of Formula (I-jj-1):

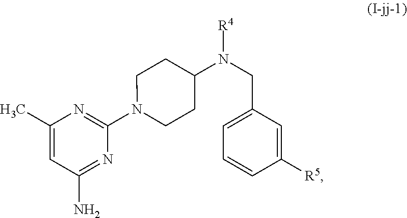

or a pharmaceutically acceptable salt, co-crystal, tautomer, stereoisomer, solvate, hydrate, polymorph, isotopically enriched derivative, or prodrug thereof, wherein R⁴ and R⁵ are as defined herein.

In certain embodiments, the compound of Formula (I) is a compound of Formula (I-jj-2):

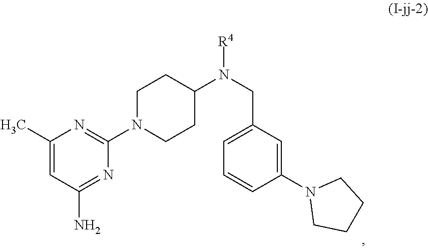

or a pharmaceutically acceptable salt, co-crystal, tautomer, stereoisomer, solvate, hydrate, polymorph, isotopically enriched derivative, or prodrug thereof, wherein R⁴ is as defined herein.

In certain embodiments, the compound of Formula (I) is a compound of the formula:

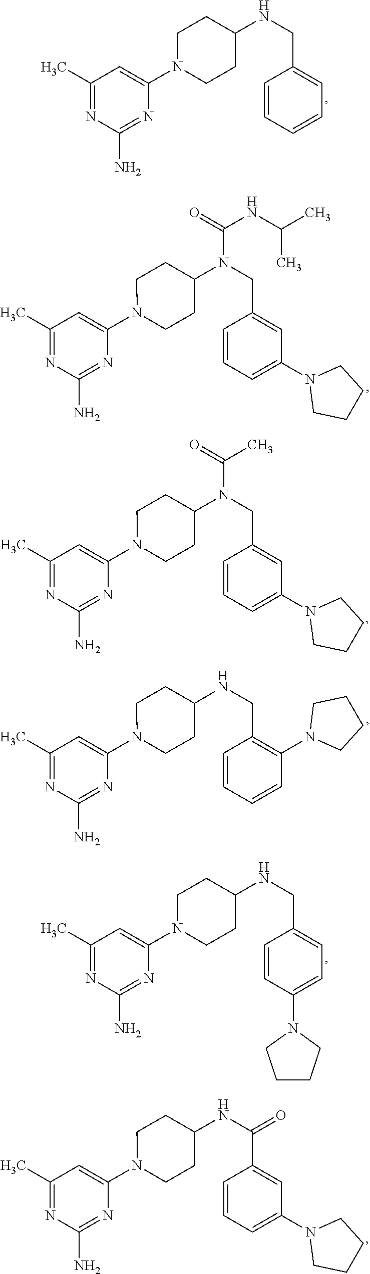

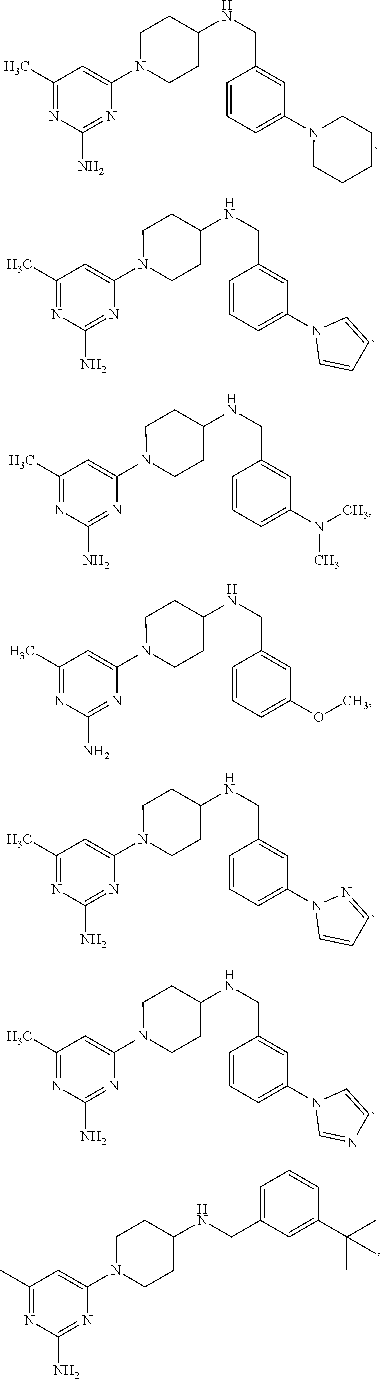

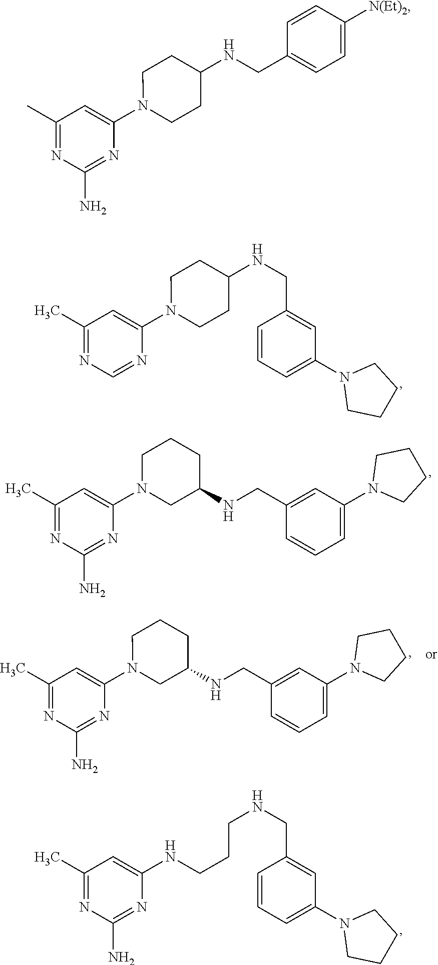

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

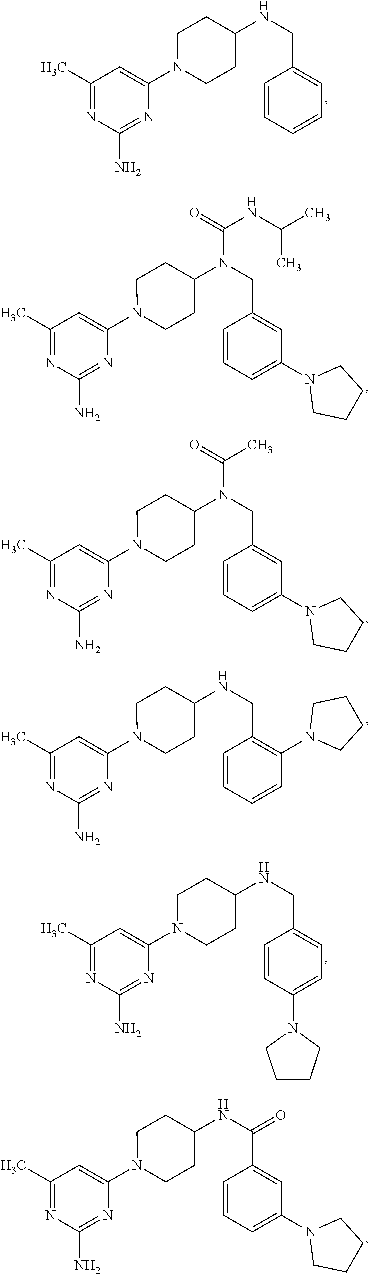

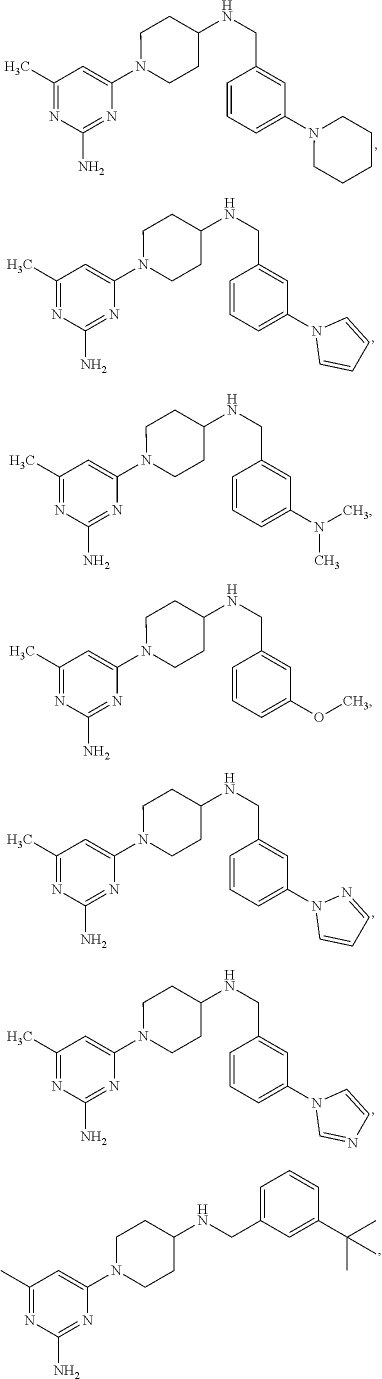

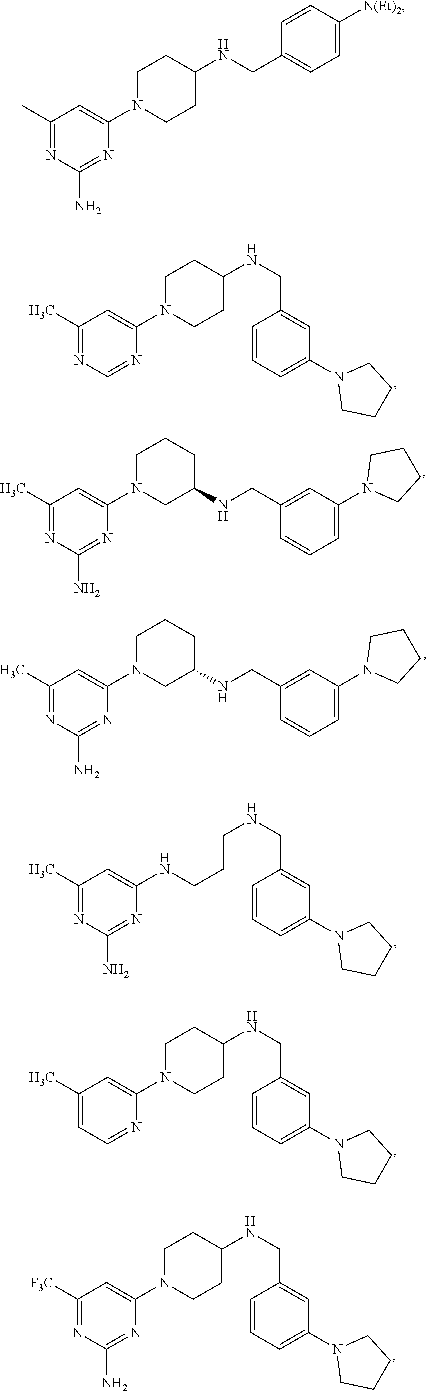

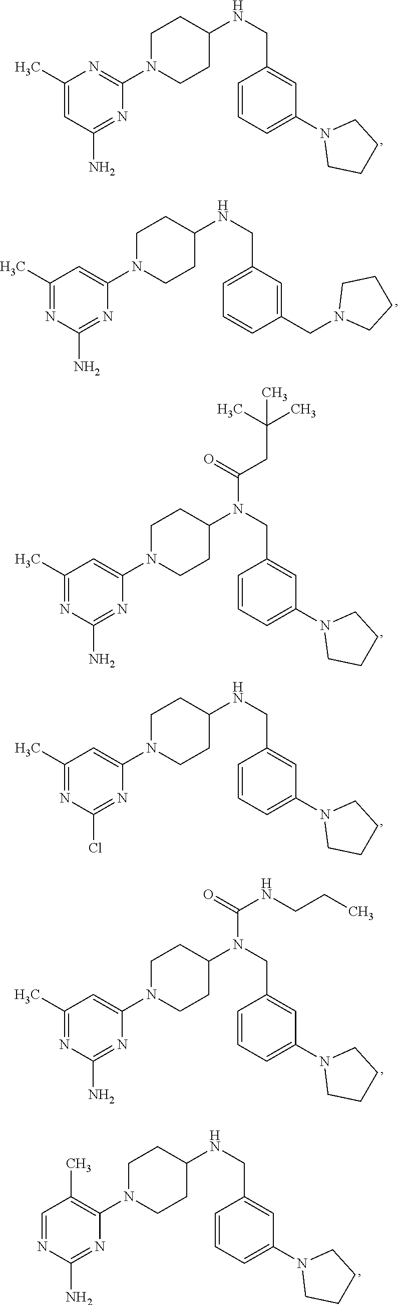 [SMILES: Cc1cc(N)nc(N2CCC(NCc3cccc(N4CCCC4)c3)CC2)n1 ; Cc1cc(N2CCC(NCc3cccc(CN4CCCC4)c3)CC2)nc(N)n1 ; Cc1cc(N2CCC(N(Cc3cccc(N4CCCC4)c3)C(=O)CC(C)(C)C)CC2)nc(N)n1 ; Cc1cc(N2CCC(NCc3cccc(N4CCCC4)c3)CC2)nc(Cl)n1 ; CCCNC(=O)N(Cc1cccc(N2CCCC2)c1)C1CCN(c2cc(C)nc(N)n2)CC1 ; Cc1cnc(N)nc1N1CCC(NCc2cccc(N3CCCC3)c2)CC1]

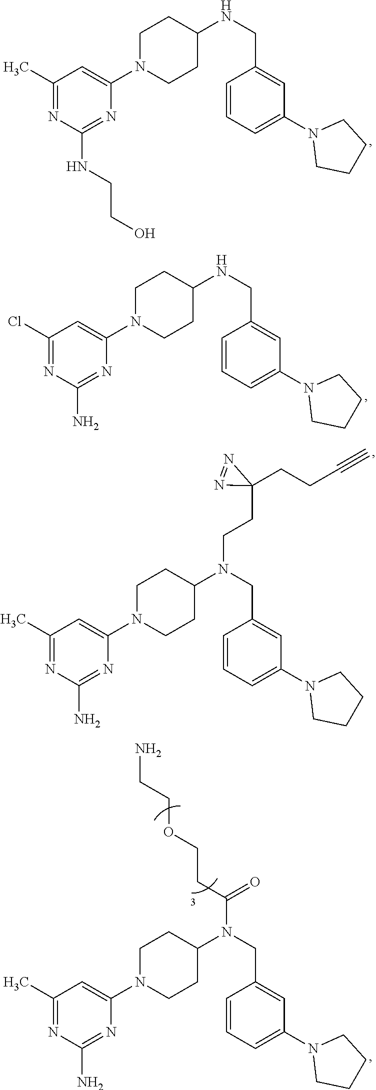

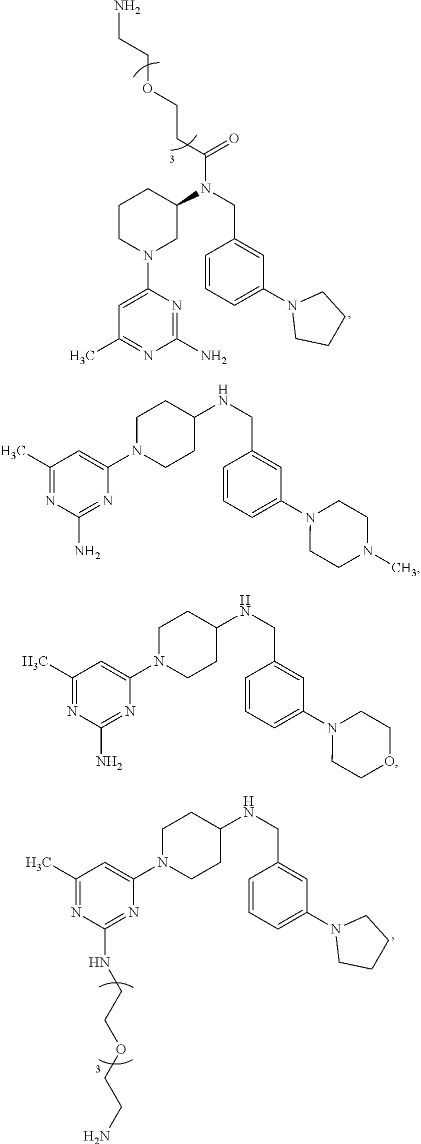

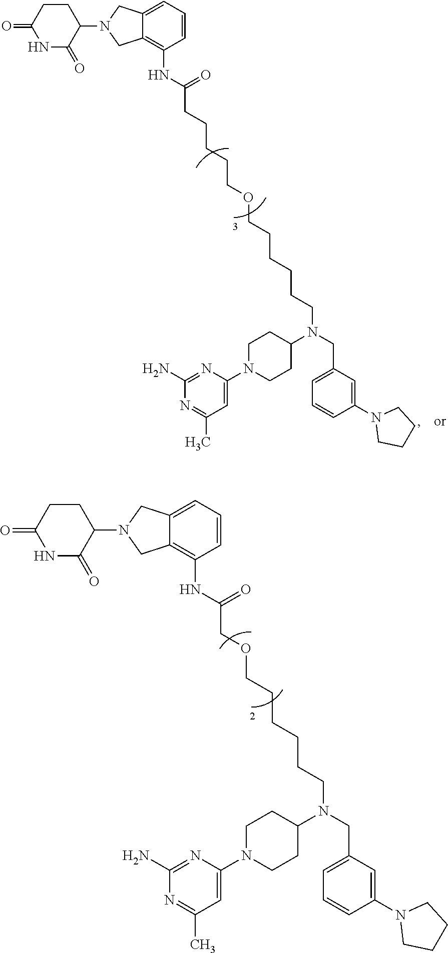

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

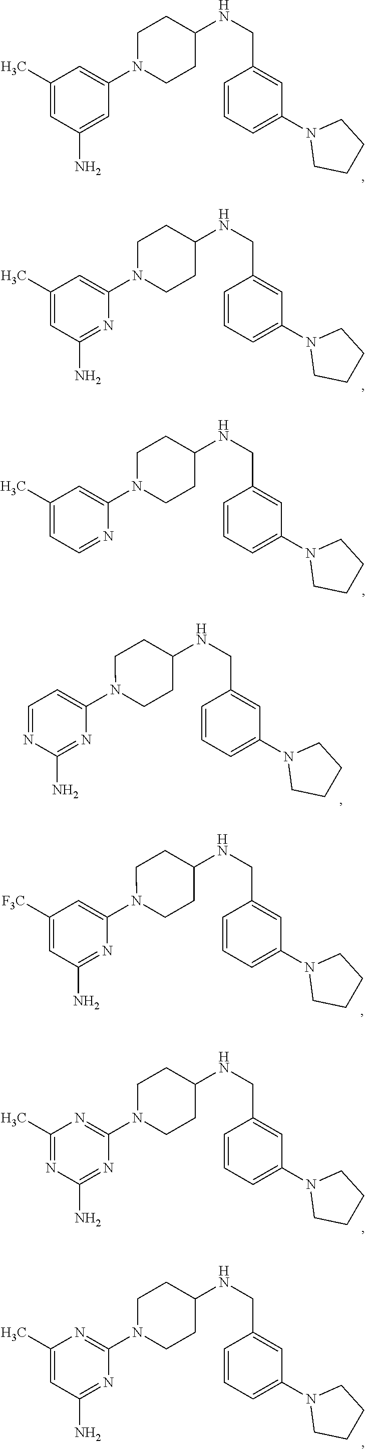

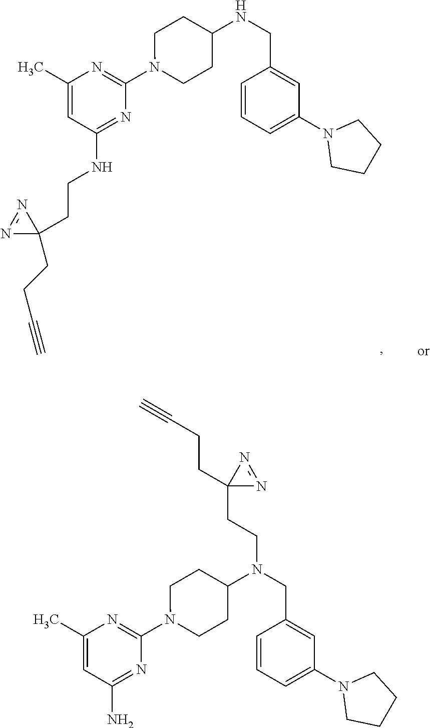 [SMILES: C#CCCC1(CCNc2cc(C)nc(N3CCC(NCc4cccc(N5CCCC5)c4)CC3)n2)N=N1 ; C#CCCC1(CCN(Cc2cccc(N3CCCC3)c2)C2CCN(c3nc(C)cc(N)n3)CC2)N=N1]

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

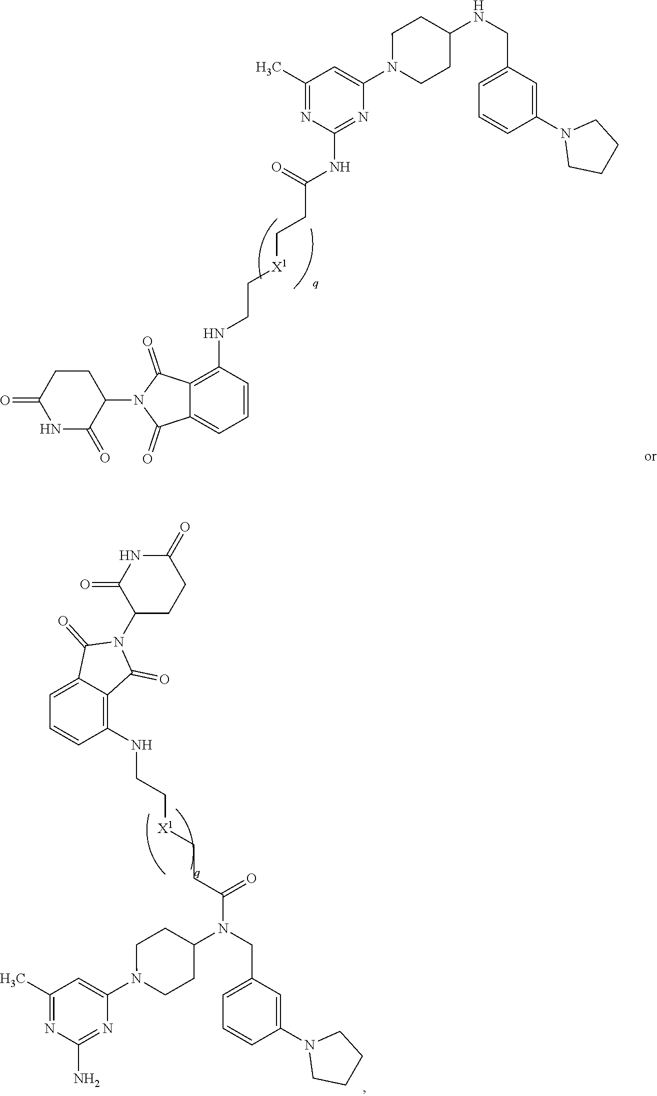

or a pharmaceutically acceptable salt, co-crystal, tautomer, stereoisomer, solvate, hydrate, polymorph, isotopically enriched derivative, or prodrug thereof, wherein X¹ is —O— or —CH₂—; and q is 1, 2, 3, or 4.

In certain embodiments, the compound of Formula (I) is a compound of the formula:

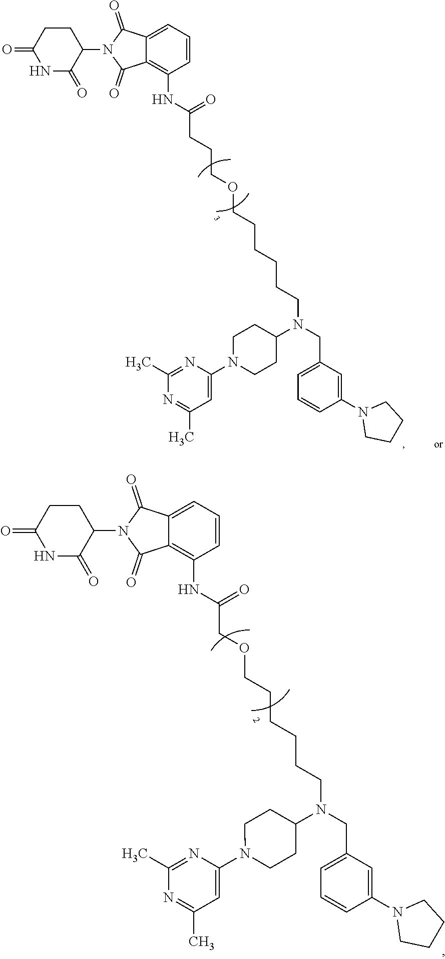

or a pharmaceutically acceptable salt, co-crystal, tautomer, stereoisomer, solvate, hydrate, polymorph, isotopically enriched derivative, or prodrug thereof.

In certain embodiments, the compound of Formula (I) binds MYB with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) inhibits MYB with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) selectively binds and/or inhibits MYB over another protein. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) disrupts and/or inhibits the interaction of a protein with MYB with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) disrupts and/or inhibits the interaction of NFIB with MYB with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) disrupts and/or inhibits the interaction of TAF12 with MYB with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) disrupts and/or inhibits the interaction of p300 with MYB with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) promotes the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of MYB at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound of Formula (I) increases the rate of MYB degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

### Pharmaceutical Compositions, Kits, and Administration

The present disclosure provides pharmaceutical compositions comprising a compound of Formula (I), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound of Formula (I) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating cancer in a subject in need thereof. In certain embodiments, the effective amount is an amount effective for preventing cancer in a subject in need thereof. In certain embodiments, the effective amount is an amount effective for treating a solid tumor or a hematological cancer in a subject in need thereof. In certain embodiments, the effective amount is an amount effective for treating a leukemia, a lymphoma, or multiple myeloma in a subject in need thereof. In certain embodiments, the effective amount is an amount effective for acute myeloid leukemia (AML) or acute lymphoblastic leukemia (ALL) in a subject in need thereof. In certain embodiments, the effective amount is an amount effective for acute myeloid leukemia (AML) in a subject in need thereof. In certain embodiments, the effective amount is an amount effective for treating rhabdomyosarcoma or adenoid cystic carcinoma in a subject in need thereof.

The present disclosure provides pharmaceutical compositions comprising a compound that inhibits the function of MYB by interacting with MYB and/or a protein associated with MYB (e.g., NFIB, p300, TAF12) for use in treating cancer in a subject in need thereof. In certain embodiments, the composition is for use in treating a solid tumor or a hematological cancer. In certain embodiments, the composition is for use in treating a leukemia, a lymphoma, or multiple myeloma. In certain embodiments, the composition is for use in treating acute myeloid leukemia (AML) or acute lymphoblastic leukemia (ALL). In certain embodiments, the composition is for use in treating acute myeloid leukemia (AML). In certain embodiments, the composition is for use in treating rhabdomyosarcoma or adenoid cystic carcinoma.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological disorder, neurodegenerative disease, and/or tauopathy). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of this disclosure can be administered to humans and other animals orally, parenterally, intracisternally, intraperitoneally, topically, bucally, or the like, depending on the disease or condition being treated.

In certain embodiments, a pharmaceutical composition comprising a compound of Formula (I) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the present disclosure are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this disclosure. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating cancer (e.g., a solid tumor or a hematological cancer) in a subject in need thereof. In certain embodiments, the kits are useful for preventing cancer (e.g., a solid tumor or a hematological cancer) in a subject in need thereof. In certain embodiments, the kits are useful for reducing the risk of developing cancer (e.g., a solid tumor or a hematological cancer) in a subject in need thereof. In certain embodiments, the kits are useful for inhibiting MYB function in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate composition.

### Methods of Use/Treatment

MYB plays a significant role in hematopoiesis regulation, and is a protooncogene that is widely deregulated in several cancers, including hematological cancers and solid tumors. Thus, inhibition of MYB function is an attractive method for the treatment of cancer.

Accordingly, use of a compound that binds and/or inhibits MYB or disrupts interactions of MYB with proteins essential for its function in cancer pathways provides a method of treating cancers that rely on MYB activity.

The present disclosure thus provides methods of inhibiting MYB function. In certain embodiments, the application provides a method of inhibiting MYB function by contacting a compound of the disclosure with MYB or a protein associated with MYB. In certain embodiments, the application provides a method of inhibiting MYB function by contacting a compound of the disclosure with MYB or a protein associated with MYB, thus disrupting an interaction between MYB and the associated protein. In certain embodiments, the application provides a method of disrupting the interaction between MYB and a protein associated with MYB. In certain embodiments, the protein associated with MYB is TAF12, NFIB, or p300. In certain embodiments, the protein associated with MYB is TAF12 or p300. In certain embodiments, the protein associated with MYB is TAF12 or NFIB. In certain embodiments, the protein associated with MYB is TAF12. In certain embodiments, the protein associated with MYB is NFIB. In certain embodiments, the protein associated with MYB is p300.

In certain embodiments, the application provides a method of inhibiting MYB function by contacting a compound of the disclosure with MYB or TAF12. In certain embodiments, the application provides a method of inhibiting MYB function by disrupting the interaction between MYB and TAF12.

In certain embodiments, the application provides a method of inhibiting MYB function by contacting a compound of the disclosure with MYB or NFIB. In certain embodiments, the application provides a method of inhibiting MYB function by disrupting the interaction between MYB and NFIB.

In certain embodiments, the application provides a method of inhibiting MYB function by contacting a compound of the disclosure with MYB or p300. In certain embodiments, the application provides a method of inhibiting MYB function by disrupting the interaction between MYB and p300.

In certain embodiments, the application provides a method of promoting the degradation of MYB or a protein associated with MYB. In certain embodiments, the protein associated with MYB is TAF12, NFIB, or p300. In certain embodiments, the protein associated with MYB is TAF12 or p300. In certain embodiments, the protein associated with MYB is TAF12 or NFIB. In certain embodiments, the protein associated with MYB is TAF12. In certain embodiments, the protein associated with MYB is NFIB. In certain embodiments, the protein associated with MYB is p300.

The present disclosure provides methods for treating cancer. In certain embodiments, the application provides a method of treating a solid tumor or a hematological cancer. In certain embodiments, the application provides a method of treating a hematological cancer. In certain embodiments, the application provides a method of treating a leukemia, a lymphoma, or multiple myeloma. In certain embodiments, the application provides a method of treating acute myeloid leukemia (AML) or acute lymphoblastic leukemia (ALL). In certain embodiments, the application provides a method of treating acute myeloid leukemia (AML).

In certain embodiments, the application provides a method of treating acute lymphoblastic leukemia (ALL). In certain embodiments, the application provides a method of treating a solid tumor. In certain embodiments, the application provides a method of treating rhabdomyosarcoma or adenoid cystic carcinoma. In certain embodiments, the application provides a method of treating rhabdomyosarcoma. In certain embodiments, the application provides a method of treating adenoid cystic carcinoma.

In certain embodiments, the methods described herein comprise administering to a subject in need thereof (e.g., a subject with a cancer) a compound that interacts with MYB, for example, a compound that is an inhibitor of MYB, a modulator of MYB, a binder of MYB, a compound that modifies MYB, a compound that promotes the degradation of MYB, or a compound that disrupts the interaction between MYB and a protein associated with MYB (e.g., NFIB, TAF12, p300). The compound may be an inhibitor of TAF12, a modulator of TAF12, a binder of TAF12, a compound that modifies TAF12, or a compound that disrupts the interaction of TAF12 with another protein. The compound may be an inhibitor of NFIB, a modulator of NFIB, a binder of NFIB, a compound that modifies NFIB, or a compound that disrupts the interaction of NFIB with another protein. The compound may be an inhibitor of p300, a modulator of p300, a binder of p300, a compound that modifies p300, or a compound that disrupts the interaction of p300 with another protein.

In certain embodiments, the methods described herein comprise administering a compound of Formula (I), or a pharmaceutically acceptable salt, co-crystal, tautomer, stereoisomer, solvate, hydrate, polymorph, isotopically enriched derivative, or prodrug, or composition thereof, to a subject in need thereof (e.g., a subject with a cancer). In some embodiments, the method comprises administering a pharmaceutical composition comprising a compound of Formula (I), or a pharmaceutically acceptable salt, co-crystal, tautomer, stereoisomer, solvate, hydrate, polymorph, isotopically enriched derivative, or prodrug, or composition thereof, to a subject in need thereof.

In certain embodiments, the methods of the disclosure comprise administering to the subject an effective amount of a compound of Formula (I), or a pharmaceutically acceptable salt, co-crystal, tautomer, stereoisomer, solvate, hydrate, polymorph, isotopically enriched derivative, or prodrug, or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formula (I), or at different times than the compound of Formula (I). For example, the compound of Formula (I) and any additional pharmaceutical agent(s) may be on the same dosing schedule or different dosing schedules. All or some doses of the compound of Formula (I) may be administered before all or some doses of an additional pharmaceutical agent, after all or some does an additional pharmaceutical agent, within a dosing schedule of an additional pharmaceutical agent, or a combination thereof. The timing of administration of the compound of Formula (I) and additional pharmaceutical agents may be different for different additional pharmaceutical agents.

In certain embodiments, the additional pharmaceutical agent comprises an agent useful in the treatment of cancer. In certain embodiments, the additional pharmaceutical agent is useful in the treatment of a solid tumor or a hematological cancer. In certain embodiments, the additional pharmaceutical agent is useful in the treatment of a hematological cancer. In certain embodiments, the additional pharmaceutical agent cancer is useful in the treatment of a leukemia, a lymphoma, or multiple myeloma. In certain embodiments, the additional pharmaceutical agent is useful in the treatment of acute myeloid leukemia (AML) or acute lymphoblastic leukemia (ALL). In certain embodiments, the additional pharmaceutical agent is useful in the treatment of acute myeloid leukemia (AML). In certain embodiments, the additional pharmaceutical agent is useful in the treatment of acute lymphoblastic leukemia (ALL). In certain embodiments, the additional pharmaceutical agent is useful in the treatment of a solid tumor. In certain embodiments, the additional pharmaceutical agent is useful in the treatment of rhabdomyosarcoma or adenoid cystic carcinoma. In certain embodiments, the additional pharmaceutical agent is useful in the treatment of rhabdomyosarcoma. In certain embodiments, the additional pharmaceutical agent is useful in the treatment of adenoid cystic carcinoma. In certain embodiments, the additional pharmaceutical agent is an anti-cancer agent. In certain embodiments, the additional pharmaceutical agent is any anti-cancer agent recited herein. In certain embodiments, the additional pharmaceutical agent is an immunotherapy. In certain embodiments, the additional pharmaceutical agent is any immunotherapy recited herein.

### EXAMPLES

In order that the present disclosure may be more fully understood, the following examples are set forth. The examples described in this application are offered to illustrate the compounds, pharmaceutical compositions, and methods provided herein and are not to be construed in any way as limiting their scope.

Initial studies showed that KI-TM1-001 binds to MYB and the MYB-NFIB fusion protein. Next, analogues of KI-TM1-001 were prepared and evaluated. Compounds of Formula (I) may be prepared using the synthetic schemes and procedures described in detail below.

All reagents were used as purchased and without purification. Reverse-phase preparative-HPLC purification was performed on an Interchim ‘PuriFlash 4125’ equipped with an Uptisphere Strategy C₁₈-HQ column (250 ×30 mm, 10 μm). Normal-phase flash chromatography was performed on an Isco ‘Rf⁺ Lumen UV-Vis’ with prepacked silica cartridges. Analytical LC-MS was performed on a Waters ‘Acquity Arc’ ultra-performance liquid chromatograph equipped with a single quadrupole mass spectrometer.

### 4-Methyl-6-(4-((3-(pyrrolidin-1-yl)benzyl)amino)piperidin-1-yl)pyrimidin-2-amine (KI-TM1-001)

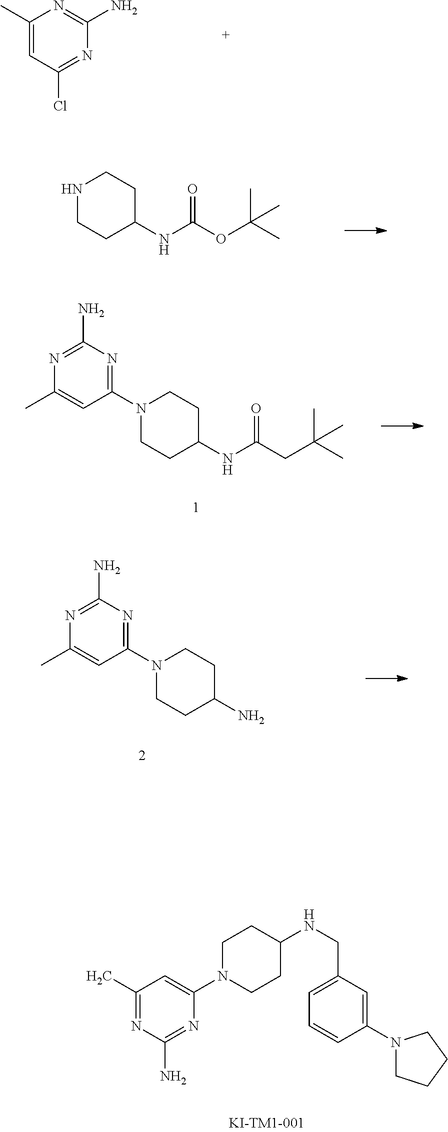

tert-Butyl (1-(2-amino-6-methylpyrimidin-4-yl)piperidin-4-yl)carbamate (1): A vial was charged with Boc-4-aminopiperidine (1.4 g, 7.0 mmol, 2.0 equiv.), 4-chloro-6-methylpyrimidin-2-amine (500 mg, 3.5 mmol, 1.0 equiv.), N,N-diisopropylethylamine (1.2 mL, 7.0 mmol, 2.0 equiv.), and isopropanol (0.1 to 0.25 M). The resultant mixture was then heated to reflux and monitored by TLC. After reaction completion, the solution was cooled to room temperature. The solution diluted with EtOAc and washed 3× with sat. NH₄Cl followed by 1×brine. The organic layer was dried with MgSO₄ and concentrated in vacuo. The crude product was purified by flash chromatography using 1→10% MeOH/DCM. Isolated 700 mg (2.3 mmol)—66% yield (88% brsm) of the title compound. ¹H NMR (CDCl₃, 500 MHz) δ 5.83 (1H, s), 4.77 (2H, s), 4.47 (1H, bs), 4.25 (2H, d, J=13.3 Hz), 3.69 (1H, bs), 3.47 (2H, s), 3.03-2.88 (2H, m), 2.20 (3H, s), 1.98 (2H, d, J=10.6 Hz), 1.44 (9H, s), 1.37-1.27 (2H, m).

2-Amino-6-(4-ammoniopiperidin-1-yl)-4-methylpyrimidin-1-ium·2 TFA (2): A vial was charged with tert-butyl (1-(2-amino-6-methylpyrimidin-4-yl)piperidin-4-yl)carbamate (250 mg, 0.813 mmol, 1.0 equiv.) followed by 1:1 TFA/DCM (0.2 M). The reaction was allowed to sit at room temperature for 1 hour, and the volatiles were then removed in vacuo. The residue was then evaporated from DCM thrice and MeOH once. The resultant bis-TFA salt was then used without further purification. Obtained 325 mg (0.75 mmol)—92% of theory—of the title compound. ¹H (CD₃OD, 500 MHz) δ 6.21 (1H, s), 4.92 (1H, d, J=11.5 Hz), 4.08 (1H, d, J=12.0 Hz), 3.36-3.27 (1H, m), 3.10-3.03 (1H, m), 2.82 (1H, t, J=11.6 Hz), 2.14 (3H, s), 2.02-1.91 (2H, m), 1.47-1.34 (2H, m). ¹³C (CD₃OD, 126 MHz) δ 162.1, 155.4, 153.8, 93.4, 47.8, 43.5, 41.6, 29.6, 29.2, 22.8, 17.4.

4-Methyl-6-(4-((3-(pyrrolidin-1-yl)benzyl)amino)piperidin-1-yl)pyrimidin-2-amine (KI-TM1-001): An oven-dried vial was charged with 2-amino-6-(4-ammoniopiperidin-1-yl)-4-methylpyrimidin-1-ium (100 mg, 230 μmol, 1.0 equiv.), 3-(pyrrolidin-1-yl)benzaldehyde (42 mg, 240 μmol, 1.1 equiv.), Et₃N (160 μL, 1.15 mmol, 5.0 equiv.), and ethanol (0.06 M). The resultant solution was then heated to reflux for 3 hours at which point the solution was cooled to room temperature. The imine solution was then slowly treated with powdered NaBH₄ (87 mg, 2.3 mmol, 10 equiv.). After off-gassing was complete, the mixture was capped and stirred overnight. In the morning, the reaction was quenched with water and stirred for 20 minutes. The solution was then extracted 3× with EtOAc. The combined organics were then dried with MgSO₄, and the volatiles were removed in vacuo. The residue was reconstituted in DMSO for prep-HPLC purification. Prep-HPLC conditions: 10→100% acetonitrile (0.1% TFA) gradient over 7 column volumes. The resulting TFA salt was cracked by dissolving the isolated material in DCM then treating with 400 wt % Na₂CO₃. After filtration of the colorless solution: isolated 70 mg (191 μmol)—83% yield of the title compound. ¹H (CD₃OD, 500 MHz) δ 7.12 (1H, t, J=7.8 Hz), 6.60 (1H, d, J=7.5 Hz), 6.58 (1H, s), 6.48 (1H, dd, J=8.1, 2.1 Hz), 5.98 (1H, s), 4.39 (2H, d, J=12.9 Hz), 3.74 (2H, s), 3.30-3.23 (4H, m), 2.87-2.73 (3H, m), 2.15 (3H, s), 2.04-1.99 (4H, m), 1.99-1.93 (2H, m), 1.37-1.25 (2H, m). ¹³C (CD₃OD, 126 MHz) δ 166.4, 164.5, 164.1, 149.9, 141.2, 130.1, 116.7, 112.9, 111.9, 93.4, 55.4, 51.7, 48.7, 44.0, 32.6, 26.4, 23.5.

### 4-(4-(Benzylamino)piperidin-1-yl)-6-methylpyrimidin-2-amine (KI-TM1-002)

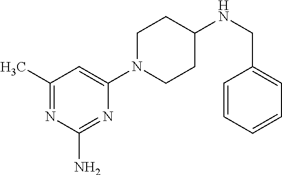 [SMILES: Cc1cc(N2CCC(NCc3ccccc3)CC2)nc(N)n1]

4-(4-(Benzylamino)piperidin-1-yl)-6-methylpyrimidin-2-amine (KI-TM1-002): KI-TM1-002 was synthesized in a manner analogous to that used to prepare KI-TM1-001 using 51 mg (116 μmol) of 2-amino-6-(4-ammoniopiperidin-1-yl)-4-methylpyrimidin-1-ium, 13 μL (128 μmol) of benzaldehyde, 81 μL (580 mmol) of Et₃N, and 44 mg (1.16 mmol) of NaBH₄. Flash chromatography conditions: 1→12% MeOH/DCM. Isolated 23 mg (77 μmol)—67% yield of the title compound. ¹H (CD₃OD, 500 MHz) δ 7.39-7.27 (m, 4H), 7.27-7.19 (m, 1H), 5.98 (s, 1H), 4.38 (d, J=12.8 Hz, 2H), 3.80 (s, 2H), 2.83 (t, J=12.2 Hz, 2H), 2.80-2.72 (m, 1H), 2.15 (s, J=12.8 Hz, 3H), 1.97 (d, J=12.1 Hz, 2H), 1.36-1.25 (m, 2H). ¹³C (CD₃OD, 126 MHz) δ 166.4, 164.5, 164.1, 140.8, 129.5, 129.5, 128.2, 93.4, 55.5, 51.2, 43.9, 32.6, 23.4.

### 1-(1-(2-Amino-6-methylpyrimidin-4-yl)piperidin-4-yl)-3-isopropyl-1-(3-(pyrrolidin-1-yl)benzyl)urea (KI-TM1-003)

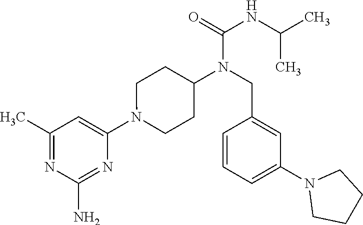 [SMILES: Cc1cc(N2CCC(N(Cc3cccc(N4CCCC4)c3)C(=O)NC(C)C)CC2)nc(N)n1]

1-(1-(2-Amino-6-methylpyrimidin-4-yl)piperidin-4-yl)-3-isopropyl-1-(3-(pyrrolidin-1-yl)benzyl)urea (KI-TM1-003): An oven-dried vial was charged with KI-TM1-001 (10 mg, 27 μmol), Et₃N (6 μL, 41 μmol), and DCM (0.1 M). The solution was cooled to 0° C. and treated with isopropyl isocyanate (3.3 μL, 30 μmol). The reaction was monitored by TLC (5% MeOH/DCM). Upon completion, the volatiles were removed in vacuo. The residue was dissolved in DMSO for prep-HPLC purification: 5→100% acetonitrile (0.1% TFA) gradient over 7 column volumes. The resulting TFA salt was cracked by dissolving the isolated material in DCM then treating with 400 wt % Na₂CO₃. After filtration of the colorless solution: isolated 8 mg (18 μmol)—66% yield of the title compound. ¹H (CD₃OD, 500 MHz) δ 7.12 (t, J=7.8 Hz, 1H), 6.51 (d, J=7.5 Hz, 1H), 6.48-6.43 (m, 2H), 5.98 (s, 1H), 4.52-4.40 (m, 3H), 4.34 (s, 2H), 3.91 (dt, J=13.1, 6.5 Hz, 1H), 3.25 (t, J=6.6 Hz, 4H), 2.92-2.81 (m, 2H), 2.15 (s, 3H), 2.05-1.98 (m, 4H), 1.77-1.70 (m, 2H), 1.61 (qd, J=12.4, 4.2 Hz, 2H), 1.07 (s, 3H), 1.06 (s, 3H). ¹³C (CD₃OD, 126 MHz) δ 166.5, 164.4, 164.1, 159.9, 149.8, 141.0, 130.3, 114.6, 111.8, 110.7, 93.4, 54.8, 48.7, 47.0, 44.8, 43.9, 31.0, 26.4, 23.4, 23.3.

### N-(1-(2-amino-6-methylpyrimidin-4-yl)piperidin-4-yl)-N-(3-(pyrrolidin-1-yl)benzyl)acetamide (KI-TM1-004)

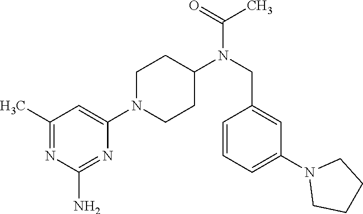 [SMILES: CC(=O)N(Cc1cccc(N2CCCC2)c1)C1CCN(c2cc(C)nc(N)n2)CC1]

N-(1-(2-Amino-6-methylpyrimidin-4-yl)piperidin-4-yl)-N-(3-(pyrrolidin-1-yl)benzyl)acetamide (KI-TM1-004): An oven-dried vial was charged with KI-TM1-001 (14 mg, 38 μmol), Et₃N (13 μL, 95 μmol), and DCM (0.1 M). The solution was cooled to 0° C. and treated with acetyl chloride (3 μL, 42 μmol). The reaction was monitored by TLC (5% MeOH/DCM). Upon completion, the volatiles were removed in vacuo. The residue was dissolved in DMSO for prep-HPLC purification: 5→100% acetonitrile (0.1% TFA) gradient over 7 column volumes. The resulting TFA salt was cracked by dissolving the isolated material in DCM then treating with 400 wt % Na₂CO₃. After filtration of the colorless solution: isolated 11 mg (27 μmol)—71% yield of the title compound. ¹H ((CD₃)₂SO, 500 MHz, 90° C.) δ 7.08 (s, 1H), 6.47-6.35 (m, 3H), 5.88 (s, 1H), 5.52 (s, 2H), 4.42 (s, 2H), 4.35 (d, J=13.1 Hz, 2H), 3.61-3.50 (m, 1H), 3.20 (bs, 4H), 2.78 (t, J=12.3 Hz, 2H), 2.06 (bs, 4H), 1.96 (bs, 4H), 1.70-1.52 (m, 4H). ¹³C ((CD₃)₂SO, 126 MHz, 90° C.) δ 169.6, 163.8, 162.2, 161.5, 147.6, 139.6, 128.4, 112.8, 109.9, 109.1, 91.4, 46.9, 42.8, 29.3, 28.7, 24.3, 22.5, 21.5.

### 4-methyl-6-(4-((2-(pyrrolidin-1-yl)benzyl)amino)piperidin-1-yl)pyrimidin-2-amine (KI-TM1-006)

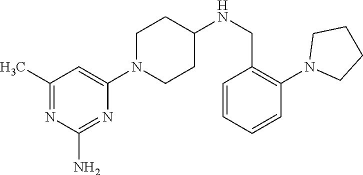 [SMILES: Cc1cc(N2CCC(NCc3ccccc3N3CCCC3)CC2)nc(N)n1]

4-methyl-6-(4-((2-(pyrrolidin-1-yl)benzyl)amino)piperidin-1-yl)pyrimidin-2-amine (KI-TM1-006): KI-TM1-006 was synthesized in a manner analogous to that used to prepare KI-TM1-001 using 25 mg (57.4 μmol) of 2-amino-6-(4-ammoniopiperidin-1-yl)-4-methylpyrimidin-1-ium, 11 mg (63.1 μmol) of 2-(pyrrolidin-1-yl)benzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (20 mg, 54.6 μmol)—95% yield. ¹H NMR (400 MHz, DMSO) δ 7.43 (dd, J=7.7, 1.6 Hz, 1H), 7.36 (s, 1H), 7.31 (td, J=7.7, 7.3, 1.6 Hz, 2H), 7.17-7.10 (m, 1H), 7.02 (td, J=7.5, 1.2 Hz, 1H), 6.36 (s, 1H), 4.49 (d, J=13.3 Hz, 2H), 4.23 (s, 2H), 3.41 (dd, J=7.5, 3.7 Hz, 1H), 3.14-3.06 (m, 4H), 3.00 (t, J=12.8 Hz, 2H), 2.20 (s, 3H), 2.16 (dd, J=12.9, 3.9 Hz, 2H), 1.95-1.84 (m, 4H), 1.52 (qd, J=12.3, 4.1 Hz, 2H). ¹³C NMR (101 MHz, DMSO) δ 161.8, 149.4, 130.6, 129.6, 124.6, 121.6, 118.6, 118.2, 115.6, 92.9, 54.3, 51.9, 44.8, 42.3, 28.0, 24.4, 19.9.

### 4-methyl-6-(4-((4-(pyrrolidin-1-yl)benzyl)amino)piperidin-1-yl)pyrimidin-2-amine (KI-TM1-007)

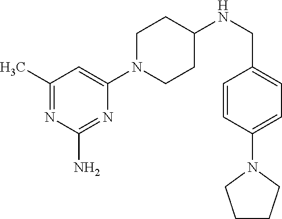 [SMILES: Cc1cc(N2CCC(NCc3ccc(N4CCCC4)cc3)CC2)nc(N)n1]

4-methyl-6-(4-((4-(pyrrolidin-1-yl)benzyl)amino)piperidin-1-yl)pyrimidin-2-amine (KI-TM1-007): KI-TM1-007 was synthesized in a manner analogous to that used to prepare KI-TM1-001 using 25 mg (57.4 μmol) of 2-amino-6-(4-ammoniopiperidin-1-yl)-4-methylpyrimidin-1-ium, 11 mg (63.1 μmol) of 4-(pyrrolidin-1-yl)benzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (12 mg, 32.7 μmol)—57% yield. ¹H NMR (400 MHz, MeOD) δ 7.19-7.11 (m, 2H), 6.58-6.50 (m, 2H), 5.97 (s, 1H), 4.38 (d, J=13.3 Hz, 2H), 3.68 (s, 2H), 3.29-3.20 (m, 4H), 2.87-2.71 (m, 3H), 2.15 (s, 4H), 2.06-1.90 (m, 7H), 1.36-1.22 (m, 4H). ¹³C NMR (101 MHz, MeOD) δ 166.4, 164.5, 164.1, 148.9, 130.5, 127.0, 112.9, 93.4, 55.3, 50.8, 48.8, 43.96, 32.5, 26.4, 23.5.

### N-(1-(2-amino-6-methylpyrimidin-4-yl)piperidin-4-yl)-3-(pyrrolidin-1-yl)benzamide (KI-TM1-008)

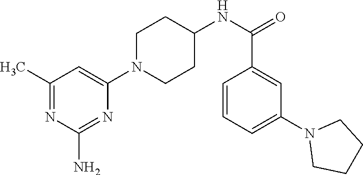 [SMILES: Cc1cc(N2CCC(NC(=O)c3cccc(N4CCCC4)c3)CC2)nc(N)n1]

N-(1-(2-amino-6-methylpyrimidin-4-yl)piperidin-4-yl)-3-(pyrrolidin-1-yl)benzamide (KI-TM1-008): KI-TM1-008 was synthesized using standard amide bond forming conditions with 25 mg (57.4 μmol) of 2-amino-6-(4-ammoniopiperidin-1-yl)-4-methylpyrimidin-1-ium, 12 mg (63.1 μmol) of 3-(pyrrolidin-1-yl)benzoic acid, 50 μL (287 mmol) of iPr₂NEt, and 33 mg (86.1 mmol) of HATU. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (21 mg, 55.2 μmol)—96% yield. ¹H NMR (400 MHz, DMSO) δ 12.78 (s, 1H), 8.16 (d, J=7.7 Hz, 1H), 7.80 (s, 2H), 7.21 (t, J=7.9 Hz, 1H), 7.04 (d, J=7.5 Hz, 1H), 6.95 (t, J=2.0 Hz, 1H), 6.65 (dd, J=8.2, 2.5 Hz, 1H), 6.50 (s, 1H), 4.76 (d, J=12.7 Hz, 1H), 4.21-4.08 (m, 2H), 3.44 (s, 3H), 3.29-3.13 (m, 3H), 2.24 (s, 3H), 1.96 (d, J=3.3 Hz, 3H), 1.93 (dd, J=9.8, 3.8 Hz, 3H), 1.52 (d, J=12.2 Hz, 2H). ¹³C NMR (101 MHz, DMSO) δ 166.5, 161.2, 155.0, 153.5, 147.5, 135.3, 128.7, 114.2, 114.0, 110.3, 93.4, 47.4, 46.1, 44.4, 42.5, 31.1, 24.9, 18.4.

### 4-methyl-6-(4-((3-(piperidin-1-yl)benzyl)amino)piperidin-1-yl)pyrimidin-2-amine (KI-TM1-009)

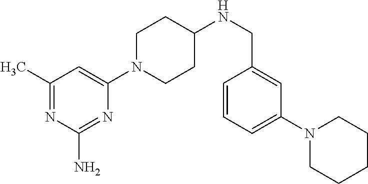 [SMILES: Cc1cc(N2CCC(NCc3cccc(N4CCCCC4)c3)CC2)nc(N)n1]

4-methyl-6-(4-((3-(piperidin-1-yl)benzyl)amino)piperidin-1-yl)pyrimidin-2-amine (KI-TM1-009): KI-TM1-009 was synthesized in a manner analogous to that used to prepare KI-TM1-001 using 25 mg (57.4 μmol) of 2-amino-6-(4-ammoniopiperidin-1-yl)-4-methylpyrimidin-1-ium, 12 mg (63.1 μmol) of 3-(piperidin-1-yl)benzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (14 mg, 36.8 μmol)—64% yield. ¹H NMR (400 MHz, MeOD) δ 7.19 (t, J=7.8 Hz, 1H), 7.00 (t, J=2.0 Hz, 1H), 6.88 (dd, J=8.1, 2.5 Hz, 1H), 6.82 (d, J=7.7 Hz, 1H), 5.98 (s, 1H), 4.39 (d, J=13.4 Hz, 2H), 3.76 (s, 2H), 3.21-3.06 (m, 4H), 2.91-2.70 (m, 3H), 2.15 (s, 3H), 2.04-1.89 (m, 2H), 1.71 (p, J=5.6 Hz, 4H), 1.65-1.53 (m, 2H), 1.39-1.23 (m, 2H). ¹³C NMR (101 MHz, MeOD) δ 166.3, 164.4, 164.0, 153.9, 141.3, 130.1, 121.1, 118.4, 117.0, 93.4, 55.5, 52.3, 51.5, 43.9, 32.5, 26.9, 25.4, 23.4.

### 4-(4-((3-(1H-pyrrol-1-yl)benzyl)amino)piperidin-1-yl)-6-methylpyrimidin-2-amine (KI-TM1-010)

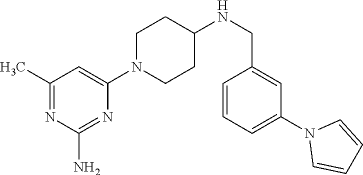 [SMILES: Cc1cc(N2CCC(NCc3cccc(-n4cccc4)c3)CC2)nc(N)n1]

4-(4-((3-(1H-pyrrol-1-yl)benzyl)amino)piperidin-1-yl)-6-methylpyrimidin-2-amine (KI-TM1-010): KI-TM1-010 was synthesized in a manner analogous to that used to prepare KI-TM1-001 using 25 mg (57.4 μmol) of 2-amino-6-(4-ammoniopiperidin-1-yl)-4-methylpyrimidin-1-ium, 12 mg (63.1 μmol) of 3-(1H-pyrrol-1-yl)benzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (19 mg, 52.4 mol)—91% yield. ¹H NMR (400 MHz, CDCl₃) δ 7.41-7.31 (m, 2H), 7.28 (ddd, J=8.0, 2.3, 1.1 Hz, 1H), 7.20 (dt, J=7.5, 1.4 Hz, 1H), 7.10 (t, J=2.2 Hz, 2H), 6.34 (t, J=2.2 Hz, 2H), 5.84 (s, 1H), 4.87 (s, 2H), 4.25 (dt, J=13.4, 4.1 Hz, 2H), 3.89 (s, 2H), 2.94 (ddd, J=13.8, 11.3, 2.8 Hz, 2H), 2.80 (tt, J=10.0, 3.9 Hz, 1H), 2.21 (s, 3H), 2.02-1.88 (m, 2H), 1.44-1.28 (m, 2H). ¹³C NMR (101 MHz, CDCl₃) δ 165.4, 163.2, 162.2, 142.5, 141.1, 129.7, 125.3, 120.2, 119.4, 119.2, 110.5, 93.1, 54.5, 50.7, 42.8, 32.4, 23.8.

### 4-(4-((3-(dimethylamino)benzyl)amino)piperidin-1-yl)-6-methylpyrimidin-2-amine (KI-TM1-011)

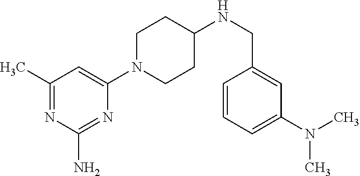

4-(4-((3-(dimethylamino)benzyl)amino)piperidin-1-yl)-6-methylpyrimidin-2-amine (KI-TM1-011): KI-TM1-011 was synthesized in a manner analogous to that used to prepare KI-TM1-001 using 25 mg (57.4 μmol) of 2-amino-6-(4-ammoniopiperidin-1-yl)-4-methylpyrimidin-1-ium, 10 mg (63.1 μmol) of 3-dimethylaminobenzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (7 mg, 20.6 μmol)—36% yield. ¹H NMR (400 MHz, MeOD) δ 7.22-7.12 (m, 1H), 6.79 (t, J=2.1 Hz, 1H), 6.69 (dd, J=8.5, 2.7 Hz, 2H), 5.99 (s, 1H), 4.39 (d, J=13.4 Hz, 2H), 3.76 (s, 2H), 2.92 (s, 6H), 2.89-2.72 (m, 3H), 2.15 (s, 3H), 1.97 (ddd, J=12.0, 4.7, 2.4 Hz, 2H), 1.38-1.25 (m, 2H). ¹³C NMR (101 MHz, MeOD) δ 165.6, 163.8, 163.4, 151.9, 140.4, 129.5, 117.6, 113.6, 112.5, 92.8, 54.9, 51.0, 43.3, 40.4, 31.8, 22.8.

### 4-(4-((3-methoxybenzyl)amino)piperidin-1-yl)-6-methylpyrimidin-2-amine (KI-TM1-012)

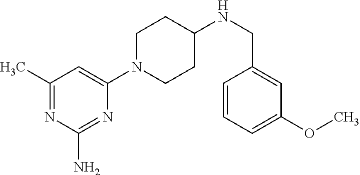

4-(4-((3-methoxybenzyl)amino)piperidin-1-yl)-6-methylpyrimidin-2-amine (KI-TM1-012): KI-TM1-012 was synthesized in a manner analogous to that used to prepare KI-TM1-001 using 25 mg (57.4 μmol) of 2-amino-6-(4-ammoniopiperidin-1-yl)-4-methylpyrimidin-1-ium, 9 mg (63.1 μmol) of 3-methoxybenzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (14 mg, 42.8 μmol)—75% yield. ¹H NMR (400 MHz, MeOD) δ 7.24 (t, J=7.9 Hz, 1H), 6.98-6.89 (m, 2H), 6.82 (ddd, J=8.2, 2.7, 1.0 Hz, 1H), 5.99 (s, 1H), 4.39 (d, J=13.4 Hz, 2H), 3.79 (s, 5H), 2.91-2.71 (m, 3H), 2.15 (s, 3H), 2.04-1.91 (m, 2H), 1.39-1.25 (m, 2H). ¹³C NMR (101 MHz, MeOD) δ 165.9, 164.4, 163.6, 161.4, 142.0, 130.5, 121.78, 115.1, 113.7, 93.5, 55.6, 55.5, 51.1, 43.9, 32.4, 23.3.

### 4-(4-((3-(1H-pyrazol-1-yl)benzyl)amino)piperidin-1-yl)-6-methylpyrimidin-2-amine (KI-TM1-013)

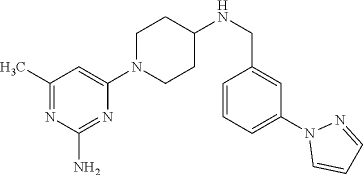 [SMILES: Cc1cc(N2CCC(NCc3cccc(-n4cccn4)c3)CC2)nc(N)n1]

4-(4-((3-(1H-pyrazol-1-yl)benzyl)amino)piperidin-1-yl)-6-methylpyrimidin-2-amine (KI-TM1-013): KI-TM1-013 was synthesized in a manner analogous to that used to prepare KI-TM1-001 using 25 mg (57.4 μmol) of 2-amino-6-(4-ammoniopiperidin-1-yl)-4-methylpyrimidin-1-ium, 11 mg (63.1 μmol) of 3-(1H-pyrazol-1-yl)benzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (19 mg, 52.3 μmol)—91% yield. ¹H NMR (400 MHz, CDCl₃) δ 7.93 (d, J=2.5 Hz, 1H), 7.71 (t, J=1.9 Hz, 2H), 7.54 (ddd, J=8.1, 2.4, 1.1 Hz, 1H), 7.39 (t, J=7.8 Hz, 1H), 7.28-7.21 (m, 1H), 6.45 (t, J=2.2 Hz, 1H), 5.83 (s, 1H), 5.04 (s, 2H), 4.26-4.20 (m, 2H), 3.89 (s, 2H), 2.94 (ddd, J=13.8, 11.3, 2.9 Hz, 2H), 2.83-2.76 (m, 1H), 2.20 (s, 3H), 1.94 (dd, J=13.0, 3.9 Hz, 2H), 1.47-1.28 (m, 2H). ¹³C NMR (101 MHz, CDCl₃) δ 164.7, 163.1, 161.8, 142.4, 141.2, 140.5, 129.6, 126.9, 126.1, 119.0, 117.8, 107.7, 93.0, 54.4, 50.7, 42.8, 32.3, 23.5.

### 4-(4-((3-(1H-imidazol-1-yl)benzyl)amino)piperidin-1-yl)-6-methylpyrimidin-2-amine (KI-TM1-014)

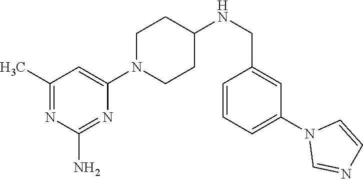 [SMILES: Cc1cc(N2CCC(NCc3cccc(-n4ccnc4)c3)CC2)nc(N)n1]

4-(4-((3-(1H-imidazol-1-yl)benzyl)amino)piperidin-1-yl)-6-methylpyrimidin-2-amine (KI-TM1-014): KI-TM1-014 was synthesized in a manner analogous to that used to prepare KI-TM1-001 using 25 mg (57.4 μmol) of 2-amino-6-(4-ammoniopiperidin-1-yl)-4-methylpyrimidin-1-ium, 11 mg (63.1 μmol) of 3-(1H-imidazol-1-yl)benzaldehyde, 40 μL (287 mmol) of Et₃N, and 7 mg (172 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (8 mg, 22.0 mol)—38% yield. ¹H NMR (400 MHz, CDCl₃) δ 7.91 (d, J=1.2 Hz, 1H), 7.54-7.44 (m, 2H), 7.44-7.30 (m, 3H), 7.26 (d, J=1.2 Hz, 1H), 5.91 (s, 1H), 5.03 (s, 2H), 4.32 (dd, J=13.3, 4.5 Hz, 2H), 3.97 (s, 2H), 3.02 (ddd, J=13.8, 11.3, 2.8 Hz, 2H), 2.87 (tt, J=10.0, 4.0 Hz, 1H), 2.72 (s, 3H), 2.38 (s, OH), 2.28 (s, 3H), 2.02 (dd, J=12.9, 3.8 Hz, 2H), 1.49-1.35 (m, 2H). ¹³C NMR (101 MHz, CDCl₃) δ 164.6, 163.1, 161.7, 143.1, 137.7, 135.7, 130.6, 130.0, 127.1, 121.1, 120.2, 118.4, 93.1, 77.5, 77.2, 76.8, 54.6, 50.6, 42.9, 32.4, 23.5.

### 4-(4-((3-(tert-Butyl)benzyl)amino)piperidin-1-yl)-6-methylpyrimidin-2-amine (KI-TM1-026)

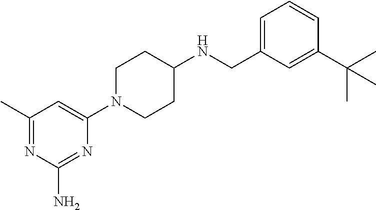 [SMILES: Cc1cc(N2CCC(NCc3cccc(C(C)(C)C)c3)CC2)nc(N)n1]

4-(4-((3-(tert-Butyl)benzyl)amino)piperidin-1-yl)-6-methylpyrimidin-2-amine (KI-TM-026): KI-TM-026 was synthesized in a manner analogous to that used to prepare KI-TM1-001 using 120 mg (0.28 mmol) of 2-amino-4-(4-ammoniopiperidin-1-yl)-6-methylpyrimidin-1-ium, 50.8 mg (0.31 mmol) of 3-(tert-butyl)benzaldehyde, 195 μL (1.40 mmol) of Et₃N, and 31.4 mg (0.84 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a white powder (81.3 mg, 0.23 mol)—82% yield. ¹H (CD₃OD, 500 MHz) δ 7.36 (1H, s), 7.25-7.19 (2H, m), 7.16-7.11 (1H, m), 5.91 (1H, s), 5.86 (2H, s), 4.15 (2H, d, J=12.9 Hz), 3.74 (2H, s), 2.94-2.82 (2H, m), 2.74-2.59 (1H, m), 2.05 (3H, s), 1.84 (2H, dd, J=12.7, 2.8 Hz), 1.27 (9H, s), 1.23-1.17 (2H, m). ¹³C (CD₃OD, 126 MHz) δ 165.4, 162.8, 162.8, 150.4, 140.5, 127.8, 125.1, 124.7, 123.3, 91.4, 53.8, 50.2, 42.1, 34.3, 31.1, 31.2, 23.6.

### 4-(4-((4-(Diethylamino)benzyl)amino)piperidin-1-yl)-6-methylpyrimidin-2-amine (KI-TM1-027)

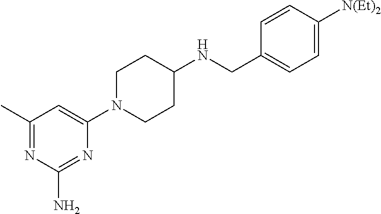

4-(4-((4-(Diethylamino)benzyl)amino)piperidin-1-yl)-6-methylpyrimidin-2-amine (KI-TM-027): KI-TM-027 was synthesized in a manner analogous to that used to prepare KI-TM1-001 using 300 mg (0.72 mmol) of 2-amino-4-(4-ammoniopiperidin-1-yl)-6-methylpyrimidin-1-ium, 138 mg (0.78 mmol) of 4-(diethylamino)benzaldehyde, 0.48 mL (3.16 mmol) of Et₃N, and 79.8 mg (2.14 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The product was extracted with dichloromethane (3×40 mL), dried over anhydrous sodium sulfate and, after removal of the solvent under reduced pressure, isolated as a colorless liquid (38.6 mg, 0.11 mmol)—15% yield. ¹H (CD₃OD, 500 MHz) δ 7.16 (2H, d, J=8.7 Hz), 6.69 (2H, d, J=8.7 Hz), 5.99 (1H, s), 4.41 (2H, d, J=12.9 Hz), 3.72 (2H, s), 3.35 (4H, q, J=7.0 Hz), 2.92-2.79 (3H, m), 2.15 (3H, s), 2.04-1.93 (2H, m), 1.39-1.26 (2H, m), 1.13 (6H, t, J=7.0 Hz). ¹³C (CD₃OD, 126 MHz) δ 166.4, 164.5, 164.0, 148.7, 130.7, 127.4, 113.7, 93.4, 55.4, 50.6, 45.5, 43.9, 32.3, 23.4, 12.8.

### (R)-4-Methyl-6-(3-((3-(pyrrolidin-1-yl)benzyl)amino)piperidin-1-yl)pyrimidin-2-amine (KI-TM1-016-(R))

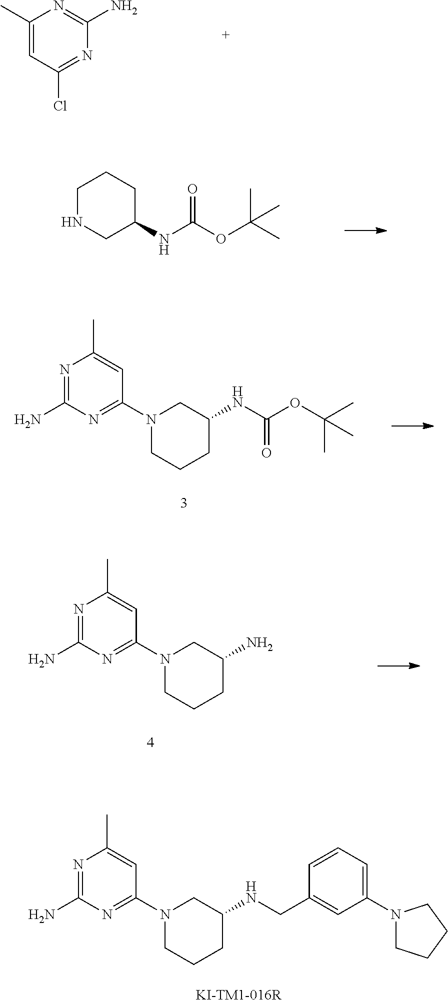

tert-Butyl (R)-(1-(2-amino-6-methylpyrimidin-4-yl)piperidin-3-yl)carbamate (3): tert-Butyl (R)-(1-(2-amino-6-methylpyrimidin-4-yl)piperidin-3-yl)carbamate was synthesized in a manner analogous to that used to prepare compound 1 using 200 mg (1.40 mmol) of 4-chloro-6-methylpyrimidin-2-amine, 556 mg (2.79 mmol) of tert-butyl (R)-piperidin-3-ylcarbamate, and 0.49 mL (2.79 mmol) of N,N-diisopropylethylamine. tert-Butyl (R)-(1-(2-amino-6-methylpyrimidin-4-yl)piperidin-3-yl)carbamate was isolated as yellowish liquid and used without further purification for the following reaction. ¹H (CDCl₃, 500 MHz) δ 5.89 (1H, s), 5.10 (2H, s), 4.61 (1H, s), 3.78 (1H, d, J=11.9 Hz), 3.73-3.58 (2H, m), 3.61-3.48 (1H, m), 3.45-3.34 (1H, m), 2.24 (3H, s), 1.98-1.87 (1H, m), 1.71 (1H, s), 1.64-1.53 (2H, m), 1.44 (9H, s).

(R)-2-Amino-6-(3-ammoniopiperidin-1-yl)-4-methylpyrimidin-1-ium·2 TFA (4): (R)-2-Amino-6-(3-ammoniopiperidin-1-yl)-4-methylpyrimidin-1-ium·2 TFA was synthesized in a manner analogous to that used to prepare compound 2 using 342 mg (1.39 mmol) of compound 3. The crude product was used without further purification for the next reaction step.

(R)-4-Methyl-6-(3-((3-(pyrrolidin-1-yl)benzyl)amino)piperidin-1-yl)pyrimidin-2-amine (KI-TM-016-(R): KI-TM-016-(R) was synthesized in a manner analogous to that used to prepare KI-TM1-001 using 540 mg (1.34 mmol) of (R)-2-amino-6-(3-ammoniopiperidin-1-yl)-4-methylpyrimidin-1-ium, 258 mg (1.48 mmol) of 3-(pyrrolidin-1-yl)benzaldehyde, 912 μL (6.70 mmol) of Et₃N, and 150 mg (4.02 mmol) of NaBH₄. The crude product was purified via flash chromatography using 1→10% MeOH/DCM as eluent. The title compound was isolated as a white powder (89.2 mg, 0.24 mmol)—18% yield. ¹H (CD₃OD, 500 MHz) δ 7.13 (1H, t, J=7.8 Hz), 6.63 (1H, d, J=7.5 Hz), 6.58 (1H, s), 6.48 (1H, dd, J=8.2, 2.1 Hz), 5.89 (1H, s), 4.33 (1H, d, J=11.6 Hz), 4.13 (1H, d, J=11.6 Hz), 3.79 (2H, s), 3.29-3.21 (4H, m), 3.00-2.91 (1H, m), 2.89-2.80 (1H, m), 2.66-2.55 (1H, m), 2.14 (3H, s), 2.06-1.96 (5H, m), 1.80-1.68 (1H, m), 1.50-1.41 (2H, m). ¹³C (CD₃OD, 126 MHz) δ 166.2, 164.5, 164.0, 149.8, 141.3, 130.2, 116.9, 113.1, 112.0, 93.4, 54.0, 52.2, 49.8, 48.7, 45.6, 32.2, 26.4, 24.7, 23.4

### (S)-4-Methyl-6-(3-((3-(pyrrolidin-1-yl)benzyl)amino)piperidin-1-yl)pyrimidin-2-amine (KI-TM1-016S))

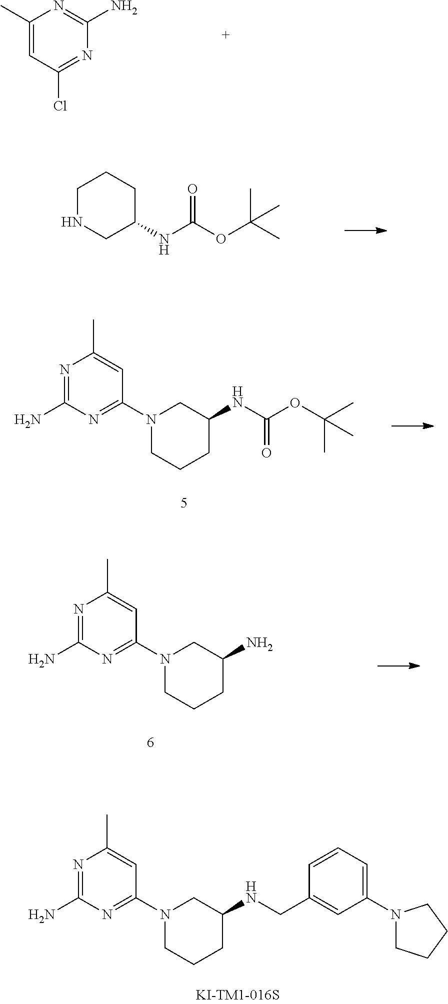

tert-Butyl (S)-(1-(2-amino-6-methylpyrimidin-4-yl)piperidin-3-yl)carbamate (5): tert-Butyl (R)-(1-(2-amino-6-methylpyrimidin-4-yl)piperidin-3-yl)carbamate was synthesized in a manner analogous to that used to prepare compound 1 using 200 mg (1.40 mmol) of 4-chloro-6-methylpyrimidin-2-amine, 556 mg (3.79 mmol) of tert-butyl (S)-piperidin-3-ylcarbamate, and 0.49 mL (3.79 mmol) of N,N-diisopropylethylamine. tert-Butyl (S)-(1-(2-amino-6-methylpyrimidin-4-yl)piperidin-3-yl)carbamate was isolated as yellowish liquid and used without further purification for the following reaction.

(S)-2-Amino-6-(3-ammoniopiperidin-1-yl)-4-methylpyrimidin-1-ium·2 TFA (6): (S)-2-Amino-6-(3-ammoniopiperidin-1-yl)-4-methylpyrimidin-1-ium·2 TFA was synthesized in a manner analogous to that used to prepare compound 2 using 150 mg (0.49 mmol) of tert-butyl (S)-(1-(2-amino-6-methylpyrimidin-4-yl)piperidin-3-yl)carbamate. The crude product was used without further purification for the next reaction step.

(S)-4-Methyl-6-(3-((3-(pyrrolidin-1-yl)benzyl)amino)piperidin-1-yl)pyrimidin-2-amine (KI-TM-016-(S): KI-TM-016-(S) was synthesized in a manner analogous to that used to prepare KI-TM1-001 using 197 mg (0.49 mmol) of (S)-2-amino-6-(3-ammoniopiperidin-1-yl)-4-methylpyrimidin-1-ium, 94.1 mg (0.54 mmol) of 3-(pyrrolidin-1-yl)benzaldehyde, 0.33 mL (2.45 mmol) of Et₃N, and 55.3 mg (1.47 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The title compound was extracted with dichloromethane (3×40 mL), dried over anhydrous sodium sulfate and, after removal of the solvent under reduced pressure, isolated as a white powder (79.2 mg, 0.21 mol)—43% yield. ¹H (CD₃OD, 500 MHz) δ 7.13 (1H, t, J=7.8 Hz), 6.62 (1H, d, J=7.4 Hz), 6.58 (1H, s), 6.47 (1H, d, J=8.1 Hz), 5.88 (1H, s), 4.32 (1H, d, J=11.6 Hz), 4.13 (1H, d, J=11.7 Hz), 3.77 (2H, s), 3.25 (4H, t, J=6.0 Hz), 2.99-2.89 (1H, m), 2.88-2.77 (1H, m), 2.65-2.54 (1H, m), 2.13 (3H, s), 2.04-1.93 (5H, m), 1.79-1.66 (1H, m), 1.49-1.40 (2H, m). ¹³C (CD₃OD, 126 MHz) δ 166.3, 164.5, 164.0, 149.8, 141.3, 130.2, 116.9, 113.1, 112.0, 93.4, 54.0, 52.2, 49.8, 48.7, 45.6, 32.3, 26.4, 24.7, 23.5.

### 6-Methyl-N⁴-(3-((3-(pyrrolidin-1-yl)benzyl)amino)propyl)pyrimidine-2,4-diamine (KI-TM1-017)

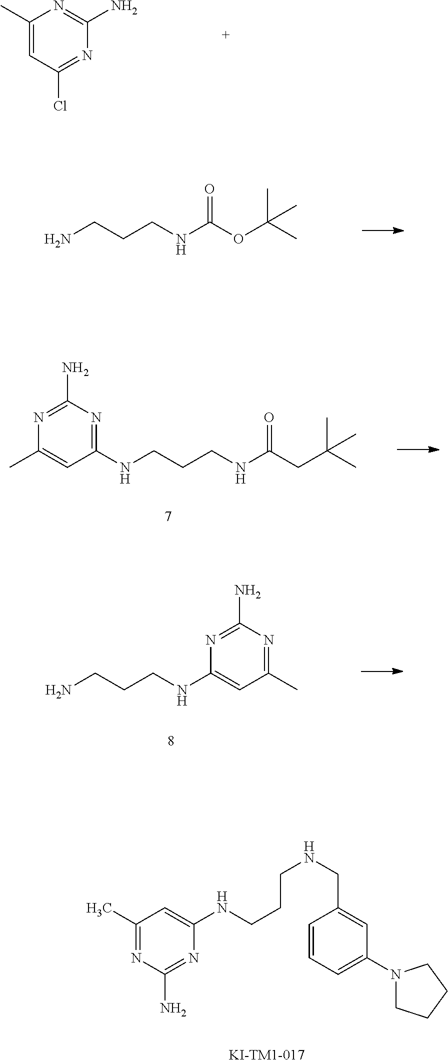

tert-Butyl (3-((2-amino-6-methylpyrimidin-4-yl)amino)propyl)carbamate (7): tert-Butyl (3-((2-amino-6-methylpyrimidin-4-yl)amino)propyl)carbamate was synthesized in a manner analogous to that used to prepare compound 1 using 150 mg (1.04 mmol) of 4-chloro-6-methylpyrimidine, 362 mg (2.08 mmol) of tert-butyl (3-aminopropyl)carbamate, and 0.36 mL (2.08 mmol) of N,N-diisopropylethylamine. tert-Butyl (3-((2-amino-6-methylpyrimidin-4-yl)amino)propyl)carbamate was isolated as a colorless liquid and used without further purification for the following reaction.

2-Amino-4-((3-ammoniopropyl)amino)-6-methylpyrimidin-1-ium·2 TFA (8): 2-Amino-4-((3-ammoniopropyl)amino)-6-methylpyrimidin-1-ium·2 TFA was synthesized in a manner analogous to that used to prepare compound 2 using 165 mg (0.32 mmol) of tert-Butyl (3-((2-amino-6-methylpyrimidin-4-yl)amino)propyl)carbamate. The crude product was used without further purification for the next reaction step.

6-Methyl-N⁴-(3-((3-(pyrrolidin-1-yl)benzyl)amino)propyl)pyrimidine-2,4-diamine (KI-TM-017): KI-TM-017 was synthesized in a manner analogous to that used to prepare KI-TM1-001 using 75.3 mg (0.18 mmol) of 2-amino-4-((3-ammoniopropyl)amino)-6-methylpyrimidin-1-ium·2 TFA, 35.1 mg (0.20 mmol) of 3-(pyrrolidin-1-yl)benzaldehyde, 0.12 mL (0.90 mmol) of Et₃N, and 20.1 mg (0.54 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The title compound was extracted with dichloromethane (3×40 mL), dried over anhydrous sodium sulfate and, after removal of the solvent under reduced pressure, isolated as a yellowish powder (20.8 mg, 63 μmol)—34% yield. ¹H (CD₃OD, 500 MHz) δ 7.15 (1H, t, J=7.8 Hz), 6.61 (1H, d, J=7.4 Hz), 6.57 (1H, s), 6.53 (1H, dd, J=8.2, 2.0 Hz), 5.71 (1H, s), 3.87 (1H, s), 3.43-3.37 (2H, m), 3.29-3.22 (4H, m), 2.79 (2H, t, J=7.0 Hz), 2.11 (3H, s), 2.04-1.99 (4H, m), 1.90-1.81 (2H, m). ¹³C (CD₃OD, 126 MHz) δ 165.6, 163.6, 149.9, 130.5, 117.0, 113.3, 112.8, 53.9, 46.2, 40.4, 38.4, 29.1, 26.4, 22.8. (3 aromatic signals (CH of pyrimidine and two C_{q}) are missing).

### 1-(6-Methylpyrimidin-4-yl)-N-(3-(pyrrolidin-1-yl)benzyl)piperidin-4-amine (KI-TM1-019)

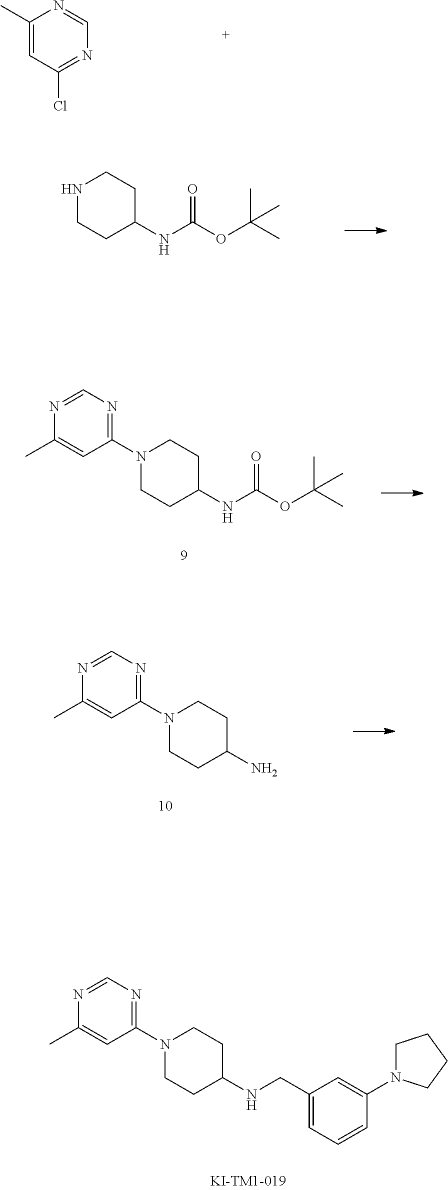

tert-Butyl (1-(6-methylpyrimidin-4-yl)piperidin-4-yl)carbamate (9): tert-Butyl (1-(6-methylpyrimidin-4-yl)piperidin-4-yl)carbamate was synthesized in a manner analogous to that used to prepare compound 1 using 500 mg (3.98 mmol) of 4-chloro-6-methylpyrimidine, 1.56 g (7.96 mmol) of tert-butyl piperidin-4-yl carbamate, and 1.39 mL (7.96 mmol) of N,N-diisopropylethylamine. tert-Butyl (1-(6-methylpyrimidin-4-yl)piperidin-4-yl)carbamate was isolated as a yellowish solid and used without further purification for the following reaction. ¹H (CDCl₃, 500 MHz) δ 8.48 (1H, s), 6.37 (1H, s), 4.48 (1H, s), 4.31 (2H, d, J=13.2 Hz), 3.72 (1H, s), 3.07-2.93 (2H, m), 2.34 (3H, s), 2.08-1.95 (2H, m), 1.44 (9H, s), 1.42-1.27 (2H, m).

6-(4-Ammoniopiperidin-1-yl)-4-methylpyrimidin-1-ium·2 TFA (10): 6-(4-Ammoniopiperidin-1-yl)-4-methylpyrimidin-1-ium·2 TFA was synthesized in a manner analogous to that used to prepare compound 2 using 1.67 g (3.98 mmol) of tert-butyl (1-(6-methylpyrimidin-4-yl)piperidin-4-yl)carbamate. The crude product was used without further purification for the next reaction step.

1-(6-Methylpyrimidin-4-yl)-N-(3-(pyrrolidin-1-yl)benzyl)piperidin-4-amine (KI-TM-019): KI-TM-019 was synthesized in a manner analogous to that used to prepare KI-TM1-001 using 272 mg (0.67 mmol) of 6-(4-ammoniopiperidin-1-yl)-4-methylpyrimidin-1-ium, 129 mg (0.74 mmol) of 3-(pyrrolidin-1-yl)benzaldehyde, 0.47 mL (3.34 mmol) of Et₃N, and 75.1 mg (2.01 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The title compound was extracted with dichloromethane (3×40 mL), dried over anhydrous sodium sulfate and, after removal of the solvent under reduced pressure, isolated as a colorless liquid (36.1 mg, 0.10 mmol)—15% yield. ¹H (CD₃OD, 500 MHz) δ 8.31 (1H, s), 7.12 (1H, t, J=7.8 Hz), 6.65 (1H, s), 6.60 (1H, d, J=7.5 Hz), 6.58 (1H, s), 6.48 (1H, dd, J=8.1, 2.1 Hz), 4.44 (2H, d, J=11.6 Hz), 3.74 (2H, s), 3.29-3.23 (4H, m), 2.98-2.87 (2H, m), 2.87-2.77 (1H, m), 2.31 (3H, s), 2.06-1.96 (6H, m), 1.40-1.27 (2H, m). ¹³C (CD₃OD, 126 MHz) δ 164.2, 161.7, 156.9, 148.4, 139.8, 128.7, 115.3, 111.5, 110.5, 101.4, 53.8, 50.3, 47.3, 42.6, 31.0, 25.0, 22.1.

### 4-(4-((3-(Pyrrolidin-1-yl)benzyl)amino)piperidin-1-yl)-6-(trifluoromethyl)pyrimidin-2-amine (KI-TM-023)

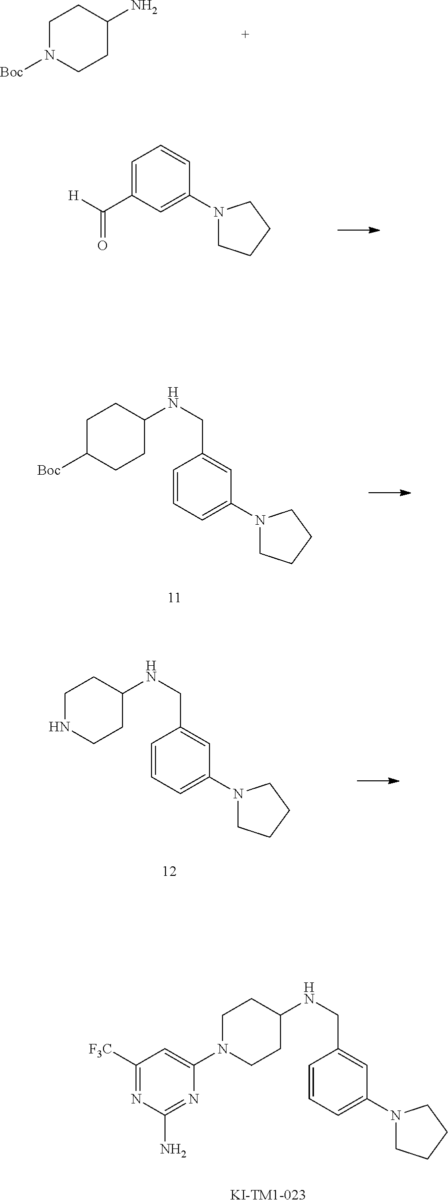 [SMILES: CC(C)(C)OC(=O)N1CCC(N)CC1 ; O=Cc1cccc(N2CCCC2)c1 ; CC(C)(C)OC(=O)C1CCC(NCc2cccc(N3CCCC3)c2)CC1 ; c1cc(CNC2CCNCC2)cc(N2CCCC2)c1 ; Nc1nc(N2CCC(NCc3cccc(N4CCCC4)c3)CC2)cc(C(F)(F)F)n1]

tert-Butyl 4-((3-(pyrrolidin-1-yl)benzyl)amino)piperidine-1-carboxylate (11): Compound 11 was synthesized in a manner analogous to that used to prepare KI-TM1-001 using 2.00 g (10.0 mmol) of tert-butyl 4-aminopiperidine-1-carboxylate, 1.93 g (11.0 mmol) of 3-(pyrrolidin-1-yl)benzaldehyde, 6.82 mL (50.0 mmol) of Et₃N, and 1.12 g (30.0 mmol) of NaBH₄. The crude product was purified via flash chromatography using 1→10% MeOH/DCM as eluent. The title compound was isolated as a white powder (3.02 g, 8.40 mmol)—84% yield. ¹H (CDCl₃, 500 MHz) δ 7.20 (t, J=7.8 Hz, 1H), 6.69-6.53 (m, 2H), 6.52-6.45 (m, 1H), 4.06 (s, 2H), 3.81 (s, 2H), 3.34-3.26 (m, 4H), 2.86-2.62 (m, 3H), 2.10-1.99 (m, 4H), 1.94 (d, J=8.2 Hz, 2H), 1.45 (s, 9H), 1.44-1.23 (m, 2H).

4-((3-(Pyrrolidin-1-yl)benzyl)amino)piperidin-1-ium·2 TFA (12): A vial was charged with tert-butyl 4-((3-(pyrrolidin-1-yl)benzyl)amino)piperidine-1-carboxylate (11) 2.0 g, 5.57 mmol, 1.0 equiv.) followed by 1:1 TFA/DCM (0.2 M). The reaction was allowed to sit at room temperature for 28 hours, and the volatiles were then removed in vacuo. The residue was then evaporated from DCM thrice and MeOH once. The resultant bis-TFA salt was then used without further purification. Obtained 2.01 g (5.35 mmol)—96% of theory—of the title compound. ¹H ((CD₃)₂SO, 500 MHz) δ 9.32 (s, 1H), 9.05 (s, 1H), 7.21 (t, J=8.0 Hz, 1H), 6.73-6.65 (m, 2H), 6.57 (d, J=8.2 Hz, 1H), 4.11 (s, 2H), 4.00 (d, J=10.5 Hz, 1H), 3.50-3.31 (m, 2H), 3.29-3.17 (m, 4H), 3.23 (t, J=6.2 Hz, 4H), 2.95 (dd, J=21.6, 10.9 Hz, 1H), 2.26 (d, J=12.7 Hz, 1H), 2.07 (d, J=10.8 Hz, 1H), 2.01-1.91 (m, 4H), 1.88-1.71 (m, 1H), 1.50-1.42 (m, 1H), 1.41 (s, 3H).

4-(4-((3-(Pyrrolidin-1-yl)benzyl)amino)piperidin-1-yl)-6-(trifluoromethyl)pyrimidin-2-amine (KI-TM-023): KI-TM-023 was synthesized in a manner analogous to that used to prepare compound 1 using 12.9 mg (0.06 mmol) of 4-chloro-6-(trifluoromethyl)pyrimidin-2-amine, 60.0 mg (0.13 mmol) of 4-((3-(pyrrolidin-1-yl)benzyl)amino)piperidin-1-ium·2 TFA and 22.7 μL (0.13 mmol) of N,N-diisopropylethylamine. The crude product was purified via flash chromatography using 0/99/1%→15/84/1% MeOH/DCM/NH₃ as eluent. The title compound was isolated as a pale-yellow powder (6.2 mg, 0.02 mmol)—36% yield. ¹H (CDCl₃, 500 MHz) δ 7.20 (t, J=7.9 Hz, 1H), 6.69 (s, 1H), 6.65 (d, J=7.4 Hz, 1H), 6.51 (dd, J=8.3, 1.5 Hz, 1H), 6.27 (s, 1H), 4.95 (s, 2H), 4.42 (s, 2H), 3.93 (s, 1H), 3.34-3.21 (m, 4H), 3.18-3.07 (m, 1H), 2.88 (t, J=12.3 Hz, 2H), 2.16 (d, J=11.5 Hz, 1H), 2.04-1.95 (m, 4H), 1.81-1.55 (m, 5H).

### 1-(4-Methylpyridin-2-yl)-N-(3-(pyrrolidin-1-yl)benzyl)piperidin-4-amine (KI-TM-021)

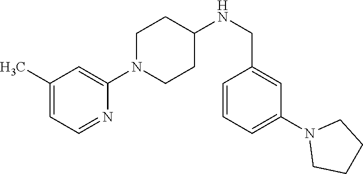 [SMILES: Cc1ccnc(N2CCC(NCc3cccc(N4CCCC4)c3)CC2)c1]

1-(4-Methylpyridin-2-yl)-N-(3-(pyrrolidin-1-yl)benzyl)piperidin-4-amine (KI-TM-021): KI-TM-021 was synthesized in a manner analogous to that used to prepare compound 1 using 22.0 mg (0.17 mmol) of 2-chloro-4-methylpyridine, 60.0 mg (0.13 mmol) of 4-((3-(pyrrolidin-1-yl)benzyl)amino)piperidin-1-ium·2 TFA and 22.7 μL (0.13 mmol) of N,N-diisopropylethylamine. The crude product was purified via flash chromatography using 0/99/1%→15/84/1% MeOH/DCM/NH₃ as eluent. The title compound was isolated as a white powder (26.4 mg, 0.08 mmol)—58% yield. ¹H ((CD₃)₂SO, 500 MHz) δ 7.07 (t, J=7.8 Hz, 1H), 6.57 (d, J=7.4 Hz, 1H), 6.53 (s, 1H), 6.40-6.33 (m, 1H), 5.91 (s, 1H), 5.83 (s, 2H), 4.14 (d, J=12.8 Hz, 2H), 3.67 (s, 2H), 3.30 (s, 2H), 3.25-3.16 (m, 4H), 2.86 (t, J=11.1 Hz, 2H), 2.67-2.60 (m, 1H), 2.05 (s, 3H), 1.94 (t, J=6.5 Hz, 4H), 1.82 (d, J=9.8 Hz, 2H), 1.24-1.09 (m, 2H).

### 6-Methyl-2-(4-((3-(pyrrolidin-1-yl)benzyl)amino)piperidin-1-yl)pyrimidin-4-amine (KI-TM-025)

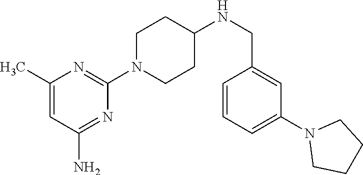 [SMILES: Cc1cc(N)nc(N2CCC(NCc3cccc(N4CCCC4)c3)CC2)n1]

6-Methyl-2-(4-((3-(pyrrolidin-1-yl)benzyl)amino)piperidin-1-yl)pyrimidin-4-amine (KI-TM-025): KI-TM-025 was synthesized in a manner analogous to that used to prepare compound 1 using 22.1 mg (0.17 mmol) of 2-chloro-6-methylpyrimidin-4-amine, 80.0 mg (0.15 mmol) of 4-((3-(pyrrolidin-1-yl)benzyl)amino)piperidin-1-ium·2 TFA and 0.10 mL (0.60 mmol) of N,N-diisopropylethylamine. The crude product was purified via flash chromatography using 0/99/1%→15/84/1% MeOH/DCM/NH₃ as eluent. The title compound was isolated as a white powder (11.8 mg, 0.03 mmol)—22% yield. ¹H (CD₃OD, 500 MHz) δ 7.15 (t, J=7.8 Hz, 1H), 6.63 (d, J=7.5 Hz, 1H), 6.61 (s, 1H), 6.51 (dd, J=8.2, 2.1 Hz, 1H), 5.70 (s, 1H), 4.68 (d, J=13.4 Hz, 2H), 3.83-3.78 (m, 2H), 3.30-3.27 (m, 3H), 2.86-2.75 (m, 3H), 2.14 (s, 3H), 2.08-2.01 (m, 4H), 1.97 (d, J=10.4 Hz, 2H), 1.41-1.31 (m, 4H).

### 4-Methyl-6-(4-((3-(pyrrolidin-1-ylmethyl)benzyl)amino)piperidin-1-yl)pyrimidin-2-amine (KI-TM-028)

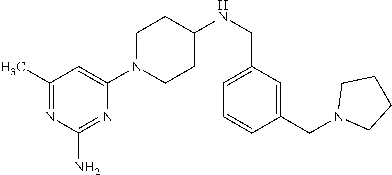 [SMILES: Cc1cc(N2CCC(NCc3cccc(CN4CCCC4)c3)CC2)nc(N)n1]

4-Methyl-6-(4-((3-(pyrrolidin-1-ylmethyl)benzyl)amino)piperidin-1-yl)pyrimidin-2-amine (KI-TM-028): KI-TM-028 was synthesized in a manner analogous to that used to prepare KI-TM1-001 using 542 mg (1.30 mmol) of 6-(4-ammoniopiperidin-1-yl)-4-methylpyrimidin-1-ium, 271 mg (1.43 mmol) of 3-(pyrrolidin-1-ylmethyl)benzaldehyde, 0.89 mL (6.50 mmol) of Et₃N, and 146 mg (3.90 mmol) of NaBH₄. The crude product was purified via flash chromatography using 0/99/1%→15/84/1% MeOH/DCM/NH₃ as eluent. The title compound was isolated as a white powder (375 mg, 0.99 mmol)—76% yield. ¹H ((CD₃)₂SO, 500 MHz) δ 7.53 (s, 1H), 7.51-7.42 (m, 3H), 6.96 (s, 2H), 6.27 (s, 1H), 4.43 (d, J=8.2 Hz, 2H), 4.13 (s, 2H), 4.06 (s, 1H), 3.06-2.82 (m, 5H), 2.17 (s, 3H), 2.11 (d, J=10.9 Hz, 2H), 1.86 (s, 4H), 1.57-1.40 (m, 2H).

### tert-Butyl (1-(2-amino-6-methylpyrimidin-4-yl)piperidin-4-yl)(3-(pyrrolidin-1-yl)benzyl)carbamate (KI-TM-029)

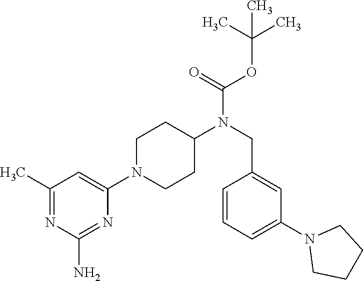 [SMILES: Cc1cc(N2CCC(N(Cc3cccc(N4CCCC4)c3)C(=O)OC(C)(C)C)CC2)nc(N)n1]

tert-Butyl (1-(2-amino-6-methylpyrimidin-4-yl)piperidin-4-yl)(3-(pyrrolidin-1-yl)benzyl)carbamate (KI-TM-029): A vial was charged with KI-TM-001 (100 mg, 0.27 mmol, 1.0 equiv.), di-tert-butyl dicarbonate (65.5 mg, 0.30 mmol, 1.1 equiv.), and tetrahydrofuran (2 mL) at 0° C. The resultant mixture was slowly warmed up to room temperature, stirred and monitored by TLC. After reaction completion, the solution was cooled to room temperature. The solvent was reduced to 50% of its volume, diluted with EtOAc and washed with 2× with H₂O followed by 1×brine. The organic layer was dried with Na₂SO₄ and concentrated in vacuo. The crude product was purified via flash chromatography using 1→10% MeOH/DCM as eluent. The title compound was isolated as a white powder (128 mg, 0.27 mmol)—100% yield. ¹H ((CD₃)₂SO, 500 MHz) δ 7.10-7.03 (m, 1H), 6.44 (d, J=7.5 Hz, 1H), 6.38 (s, 2H), 6.09 (s, 2H), 5.97 (s, 1H), 4.34 (s, 2H), 4.28 (s, 2H), 3.21-3.12 (m, 5H), 2.73 (d, J=6.0 Hz, 2H), 2.06 (s, 3H), 1.97-1.89 (m, 4H), 1.56 (s, 3H), 1.48 (s, 2H), 1.37 (d, J=5.5 Hz, 9H).

### 1-(2-Chloro-6-methylpyrimidin-4-yl)-N-(3-(pyrrolidin-1-yl)benzyl)piperidin-4-amine (KI-TM-033)

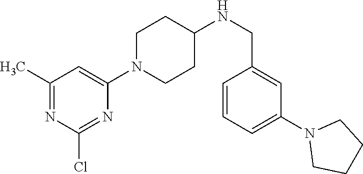 [SMILES: Cc1cc(N2CCC(NCc3cccc(N4CCCC4)c3)CC2)nc(Cl)n1]

1-(2-Chloro-6-methylpyrimidin-4-yl)-N-(3-(pyrrolidin-1-yl)benzyl)piperidin-4-amine (KI-TM-033): KI-TM-033 was synthesized in a manner analogous to that used to prepare compound 1, except for the usage of DMF, using 22.1 mg (0.17 mmol) of 2-chloro-6-methylpyrimidin-4-amine, 80.0 mg (0.15 mmol) of 4-((3-(pyrrolidin-1-yl)benzyl)amino)piperidin-1-ium·2 TFA and 0.10 mL (0.60 mmol) of N,N-diisopropylethylamine. The crude product was purified via flash chromatography using 0/99/1%→15/84/1% MeOH/DCM/NH₃ as eluent. The title compound was isolated as a white powder (250 mg, 0.65 mmol)—74% yield. ¹H ((CD₃)₂SO) δ 7.07 (t, J=7.8 Hz, 1H), 6.72 (s, 1H), 6.57 (d, J=7.4 Hz, 1H), 6.53 (s, 1H), 6.38 (dd, J=8.0, 1.6 Hz, 1H), 4.12 (s, 2H), 3.67 (s, 2H), 3.26-3.16 (m, 4H), 3.12-3.01 (m, 2H), 2.75-2.64 (m, 1H), 2.23 (s, 3H), 2.00-1.90 (m, 4H), 1.92-1.82 (m, 2H), 1.31-1.18 (m, 2H).

### 1-(1-(2-Amino-6-methylpyrimidin-4-yl)piperidin-4-yl)-3-propyl-1-(3-(pyrrolidin-1-yl)benzyl)urea (KI-TM-034)

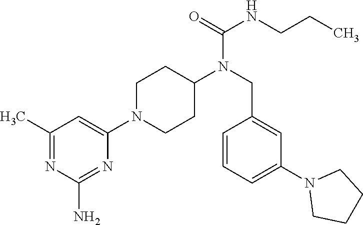 [SMILES: CCCNC(=O)N(Cc1cccc(N2CCCC2)c1)C1CCN(c2cc(C)nc(N)n2)CC1]

1-(1-(2-Amino-6-methylpyrimidin-4-yl)piperidin-4-yl)-3-propyl-1-(3-(pyrrolidin-1-yl)benzyl)urea (KI-TM-034): A vial was charged with KI-TM-001 (90.1 mg, 0.25 mmol, 1.0 equiv.), n-propyl isocyanate (65.5 mg, 0.25 mmol, 1.0 equiv.), and pyridine (1.25 mL) at room temperature for 2 h. After reaction completion, DCM was added, and the solvents were removed under reduced pressure. The crude product was purified via flash chromatography using 1→10% MeOH/DCM as eluent. The title compound was isolated as a white powder (66.3 mg, 0.15 mmol)—61% yield. ¹H ((CD₃)₂SO) δ 7.06 (t, J=8.0 Hz, 1H), 6.43 (d, J=7.4 Hz, 1H), 6.36 (d, J=6.5 Hz, 3H), 6.20 (s, 1H), 4.41 (d, J=41.3 Hz, 2H), 4.30 (s, 2H), 3.16 (s, 4H), 2.98 (dt, J=22.8, 9.3 Hz, 4H), 2.17 (s, 3H), 1.93 (s, 4H), 1.63 (d, J=10.8 Hz, 2H), 1.60-1.44 (m, 2H), 1.44-1.32 (m, 3H), 1.20 (d, J=24.1 Hz, 1H), 0.78 (t, J=7.4 Hz, 3H).

### 5-Methyl-4-(4-((3-(pyrrolidin-1-yl)benzyl)amino)piperidin-1-yl)pyrimidin-2-amine (KI-TM-036)

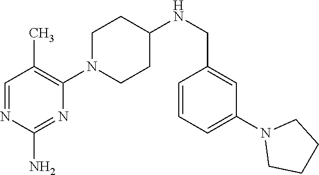 [SMILES: Cc1cnc(N)nc1N1CCC(NCc2cccc(N3CCCC3)c2)CC1]

5-Methyl-4-(4-((3-(pyrrolidin-1-yl)benzyl)amino)piperidin-1-yl)pyrimidin-2-amine (KI-TM-036): KI-TM-036 was synthesized in a manner analogous to that used to prepare compound 1, except for the usage of DMF, 22.1 mg (0.17 mmol) of 4-chloro-5-methylpyrimidin-2-amine, 80.1 mg (0.15 mmol) of 4-((3-(pyrrolidin-1-yl)benzyl)amino)piperidin-1-ium·2 TFA and 0.10 mL (0.6 mmol) of N,N-diisopropylethylamine. The crude product was purified via flash chromatography using 0/99/1%→15/84/1% MeOH/DCM/NH₃ as eluent. The title compound was isolated as a yellow powder (51.0 mg, 0.13 mmol)—88% yield. ¹H (CD₃OD, 500 MHz) δ 7.65 (s, 1H), 7.15 (t, J=7.8 Hz, 1H), 6.66-6.59 (m, 2H), 6.50 (dd, J=8.1, 2.2 Hz, 1H), 4.04 (d, J=13.3 Hz, 2H), 3.77 (s, 2H), 3.32-3.26 (m, 4H), 2.88-2.79 (m, 2H), 2.79-2.69 (m, 1H), 2.11 (s, 3H), 2.06-2.01 (m, 5H), 2.00 (s, 1H), 1.49 (qd, J=12.5, 3.8 Hz, 2H).

### 2-((4-Methyl-6-(4-((3-(pyrrolidin-1-yl)benzyl)amino)piperidin-1-yl)pyrimidin-2-yl)amino)ethan-1-ol (KI-TM-037)

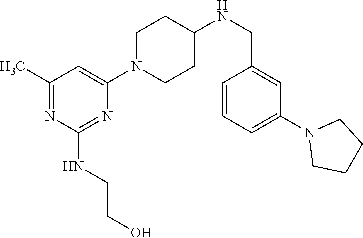 [SMILES: Cc1cc(N2CCC(NCc3cccc(N4CCCC4)c3)CC2)nc(NCCO)n1]

2-((4-Methyl-6-(4-((3-(pyrrolidin-1-yl)benzyl)amino)piperidin-1-yl)pyrimidin-2-yl)amino)ethan-1-ol (KI-TM-037): A vial was charged with KI-TM-001 (45.1 mg, 0.12 mmol, 1.0 equiv.), ethanolamine (22.2 μL, 0.36 mmol, 3.0 equiv.), 0.08 mL (0.48 mmol, 4 equiv) of N,N-diisopropylethylamine, DMF (0.13 M) at 100° C. and monitored by TLC. After reaction completion, the mixture was diluted with EtOAc and water and extracted with EtOAc. The organic layer was washed with water. The crude product was purified via flash chromatography using 0/99/1%→10/89/1% MeOH/DCM/NH₃ as eluent. The title compound was isolated as a yellow oil (35.5 mg, 0.87 mmol)—73% yield. ¹H (CD₃OD, 500 MHz) δ 7.15 (t, J=7.8 Hz, 1H), 6.67-6.56 (m, 2H), 6.50 (dd, J=8.1, 2.0 Hz, 1H), 5.97 (s, 1H), 5.51 (s, 1H), 4.44 (d, J=12.1 Hz, 2H), 3.78 (s, 2H), 3.69 (d, J=5.7 Hz, 2H), 3.47 (d, J=5.7 Hz, 2H), 3.32-3.24 (m, 5H), 2.93-2.78 (m, 3H), 2.17 (s, 3H), 2.07-1.96 (m, 6H), 1.41-1.28 (m, 4H).

### 4-Chloro-6-(4-((3-(pyrrolidin-1-yl)benzyl)amino)piperidin-1-yl)pyrimidin-2-amine (KI-TM-038)

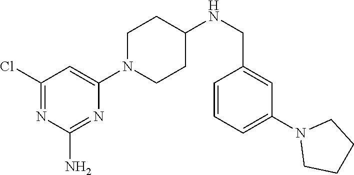 [SMILES: Nc1nc(Cl)cc(N2CCC(NCc3cccc(N4CCCC4)c3)CC2)n1]

4-Chloro-6-(4-((3-(pyrrolidin-1-yl)benzyl)amino)piperidin-1-yl)pyrimidin-2-amine (KI-TM-038): KI-TM-038 was synthesized in a manner analogous to that used to prepare compound 1, except for the usage of DMF, using 40.6 mg (0.28 mmol) of 4,6-dichloropyrimidin-2-amine, 100 mg (0.26 mmol) of 4-((3-(pyrrolidin-1-yl)benzyl)amino)piperidin-1-ium·2 TFA and 0.18 mL (1.04 mmol) of N,N-diisopropylethylamine. Prep-HPLC conditions: 10→100% acetonitrile (0.1% TFA) gradient over 7 column volumes. The resulting product was received as TFA salt which was treated with aqueous saturated sodium carbonate solution. The title compound was extracted with dichloromethane (3×40 mL), dried over anhydrous sodium sulfate and, after removal of the solvent under reduced pressure, isolated as a colorless oil (13.0 mg, 0.03 mmol)—13% yield. ¹H ((CD₃)₂SO) δ 7.07 (t, J=7.8 Hz, 1H), 6.56 (d, J=7.5 Hz, 1H), 6.53 (s, 1H), 6.43-6.36 (m, 3H), 6.08 (s, 1H), 5.75 (s, 1H), 4.13 (s, 1H), 3.66 (s, 2H), 3.24-3.17 (m, 4H), 2.98-2.87 (m, 2H), 2.71-2.60 (m, 1H), 1.97-1.91 (m, 4H), 1.82 (dd, J=12.7, 2.9 Hz, 2H), 1.24-1.13 (m, 2H).

### 4-Methyl-6-(4-((3-(4-methylpiperazin-1-yl)benzyl)amino)piperidin-1-yl)pyrimidin-2-amine (KI-TM-039)

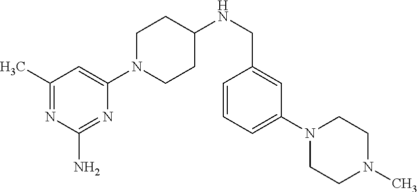

4-Methyl-6-(4-((3-(4-methylpiperazin-1-yl)benzyl)amino)piperidin-1-yl)pyrimidin-2-amine (KI-TM-039): KI-TM-039 was synthesized in a manner analogous to that used to prepare KI-TM1-001 using 80.0 mg (0.19 mmol) of 6-(4-ammoniopiperidin-1-yl)-4-methylpyrimidin-1-ium, 42.7 mg (0.21 mmol) of 3-(4-methylpiperazin-1-yl)benzaldehyde, 0.13 mL (0.95 mmol) of Et₃N, and 21.3 mg (0.57 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The title compound was extracted with dichloromethane (3×40 mL), dried over anhydrous sodium sulfate and, after removal of the solvent under reduced pressure, isolated as a grey solid (32.1 mg, 0.08 mmol)—43% yield. ¹H ((CD₃)₂SO) δ 7.24 (t, J=7.9 Hz, 1H), 7.02 (s, 1H), 6.91 (dd, J=8.2, 2.2 Hz, 1H), 6.87 (d, J=7.5 Hz, 1H), 6.03 (s, 1H), 4.43 (d, J=13.0 Hz, 2H), 3.82 (s, 2H), 3.26-3.20 (m, 4H), 2.92-2.80 (m, 3H), 2.67-2.61 (m, 4H), 2.37 (s, 3H), 2.18 (s, 3H), 2.01 (d, J=10.5 Hz, 2H), 1.40-1.30 (m, 3H).

### 4-Methyl-6-(4-((3-morpholinobenzyl)amino)piperidin-1-yl)pyrimidin-2-amine (KI-TM-040)

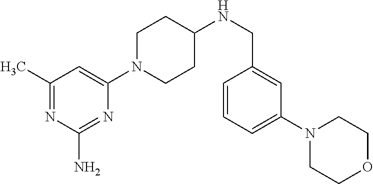 [SMILES: Cc1cc(N2CCC(NCc3cccc(N4CCOCC4)c3)CC2)nc(N)n1]

4-Methyl-6-(4-((3-morpholinobenzyl)amino)piperidin-1-yl)pyrimidin-2-amine (KI-TM-040): KI-TM-040 was synthesized in a manner analogous to that used to prepare KI-TM1-001 using 50.0 mg (0.12 mmol) of 6-(4-ammoniopiperidin-1-yl)-4-methylpyrimidin-1-ium, 24.9 mg (0.13 mmol) of 3-morpholinobenzaldehyde, 0.08 mL (0.59 mmol) of Et₃N, and 13.5 mg (0.36 mmol) of NaBH₄. Prep-HPLC conditions: 10→100% acetonitrile (0.1% TFA) gradient over 7 column volumes. The resulting product was received as TFA salt which was treated with aqueous saturated sodium carbonate solution. The title compound was extracted with dichloromethane (3×40 mL), dried over anhydrous sodium sulfate and, after removal of the solvent under reduced pressure, isolated as a colorless liquid (32.1 mg, 0.08 mmol)—70% yield. ¹H (CD₃OD, 500 MHz) δ 7.25 (d, J=7.9 Hz, 1H), 7.02 (s, 1H), 6.95-6.85 (m, 2H), 6.05 (s, 1H), 4.45 (d, J=12.8 Hz, 2H), 3.88-3.81 (m, 6H), 3.19-3.13 (m, 4H), 2.95-2.84 (m, 3H), 2.19 (s, 3H), 2.07-1.99 (m, 2H), 1.42-1.31 (m, 4H).

### 4-(4-((2-(3-(But-3-yn-1-yl)-3H-diazirin-3-yl)ethyl)(3-(pyrrolidin-1-yl)benzyl)amino)-piperidin-1-yl)-6-methylpyrimidin-2-amine (KI-TM-030)

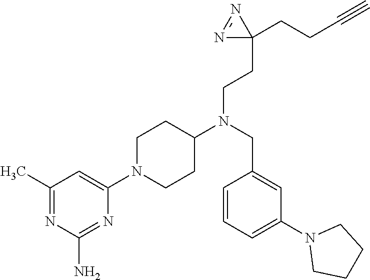 [SMILES: C#CCCC1(CCN(Cc2cccc(N3CCCC3)c2)C2CCN(c3cc(C)nc(N)n3)CC2)N=N1]

4-(4-((2-(3-(But-3-yn-1-yl)-3H-diazirin-3-yl)ethyl)(3-(pyrrolidin-1-yl)benzyl)amino)-piperidin-1-yl)-6-methylpyrimidin-2-amine (KI-TM-030): A vial was charged with KI-TM-001 (23.6 mg, 0.07 mmol, 1.0 equiv.), 3-(but-3-yn-1-yl)-3-(2-iodoethyl)-3H-diazirine (16.0 mg, 0.07 mmol, 3.0 equiv.), 13.5 μL (0.10 mmol, 1.5 equiv) of triethylamine, DMF (0.08 M) at 40° C. and monitored by TLC. After reaction completion, the mixture was diluted with EtOAc and water and extracted with EtOAc. The organic layer was washed with water. The crude product was purified via flash chromatography using 0/99/1%→10/89/1% MeOH/DCM/NH₃ as eluent. The title compound was isolated as a yellow oil (6.35 mg, 0.01 mmol)—21% yield. ¹H (CD₃OD, 500 MHz) δ 7.20 (t, J=7.8 Hz, 3H), 6.69-6.61 (m, 6H), 6.57 (dd, J=8.2, 2.0 Hz, 3H), 6.10 (s, 3H), 4.52 (d, J=12.8 Hz, 7H), 3.95 (s, 6H), 3.31-3.27 (m, 12H), 3.16-3.06 (m, 4H), 2.98-2.88 (m, 7H), 2.21 (s, 10H), 2.14-2.07 (m, 7H), 2.07-2.02 (m, 13H), 1.93 (s, 4H), 1.48-1.39 (m, 9H), 1.38-1.26 (m, 15H), 1.06-0.82 (m, 7H).

### N-(1-(2-Amino-6-methylpyrimidin-4-yl)piperidin-4-yl)-3-(2-(2-(2-aminoethoxy)ethoxy)-ethoxy)-N-(3-(pyrrolidin-1-yl)benzyl)propenamide (KI-TM-031)

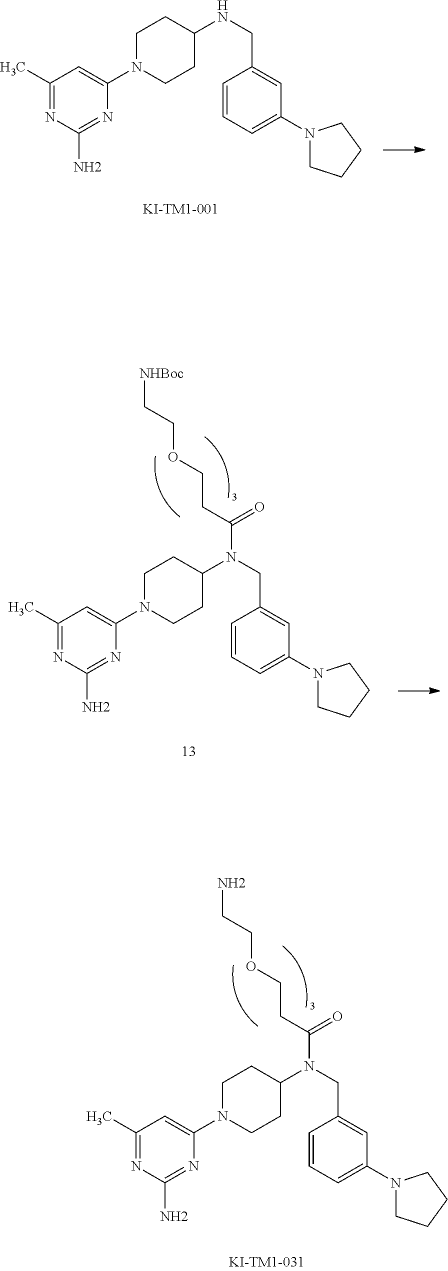

tert-Butyl (2-(1-(2-amino-6-methylpyrimidin-4-yl)piperidin-4-yl)-3-oxo-1-(3-(pyrrolidin-1-yl)phenyl)-6,9,12-trioxa-2-azatetradecan-14-yl)carbamate (13): A vial was charged with 2,2-dimethyl-4-oxo-3,8,11,14-tetraoxa-5-azaheptadecan-17-oic acid (80.4 mg, 0.22 mmol, 1.0 equiv.), N,N′-dicyclohexylcarbodiimide (45.4 mg, 0.22 mmol, 1.0 equiv.), DCM (0.22 M), 1-hydroxybenzotriazole (35.1 mg, 0.26 mmol, 1.2 equiv.) and DMF (0.73 M) at 0° C. The mixture was warmed up to room temperature and stirred for 0.5 h. KI-TM-001 (80.0 mg, 0.22 mmol, 1.0 equiv.) and DCM (1.1 M) were added, and the resulting mixture was stirred at room temperature while monitored by TLC. After reaction completion, the precipitated urea derivatives were removed by filtration. The filtrate was diluted with DCM and and washed with aqueous saturated bicarbonate solution and extracted 3× with DCM. The combined organic layers were washed with brine. The crude product was purified via flash chromatography using 0/99/1%→10/89/1% MeOH/DCM/NH₃ as eluent. The title compound was isolated as a white solid (75.3 mg, 0.11 mmol)—50% yield. ¹H (CD₃OD, 500 MHz) δ 7.17-7.02 (m, 1H), 6.50-6.37 (m, 3H), 5.97 (s, 1H), 4.53 (d, J=6.2 Hz, 2H), 4.50-4.41 (m, 2H), 3.87 (t, J=6.1 Hz, 1H), 3.76 (t, J=6.3 Hz, 1H), 3.66 (s, 1H), 3.64-3.54 (m, 7H), 3.52-3.43 (m, 2H), 3.27-3.16 (m, 6H), 2.92-2.81 (m, 3H), 2.61 (t, J=6.3 Hz, 1H), 2.14 (d, J=3.7 Hz, 3H), 2.04-1.96 (m, 4H), 1.80-1.53 (m, 4H), 1.42 (s, 9H).

N-(1-(2-Amino-6-methylpyrimidin-4-yl)piperidin-4-yl)-3-(2-(2-(2-aminoethoxy)ethoxy)-ethoxy)-N-(3-(pyrrolidin-1-yl)benzyl)propenamide (KI-TM-031): A vial was charged compound (13) 30 mg, 0.04 mmol, 1.0 equiv.) followed by 1:1 TFA/DCM (0.2 M). The reaction was allowed to sit at room temperature for 28 hours, and the volatiles were then removed in vacuo. The residue was then evaporated from DCM thrice and MeOH once. The crude product was purified via flash chromatography using 0/99/1%→15/84/1% MeOH/DCM/NH₃ as eluent. The title compound was isolated as a white solid (23.2 mg, 0.04 mmol)—91% yield. ¹H (CD₃OD, 500 MHz) δ 7.16-7.01 (m, 1H), 6.50-6.37 (m, 3H), 5.96 (s, 1H), 5.49 (s, 1H), 4.66-4.58 (m, 1H), 4.57-4.51 (m, 2H), 4.46 (d, J=10.7 Hz, 2H), 3.89-3.72 (m, 2H), 3.68-3.48 (m, 10H), 3.28-3.18 (m, 4H), 2.91-2.80 (m, 3H), 2.76 (t, J=5.3 Hz, 2H), 2.60 (t, J=6.3 Hz, 1H), 2.14 (d, J=4.3 Hz, 3H), 2.05-1.96 (m, 4H), 1.81-1.57 (m, 4H).

### N-(1-(2-Amino-6-methylpyrimidin-4-yl)piperidin-3-yl)-3-(2-(2-(2-aminoethoxy)ethoxy)-ethoxy)-N-(3-(pyrrolidin-1-yl)benzyl)propenamide (KI-TM1-32)

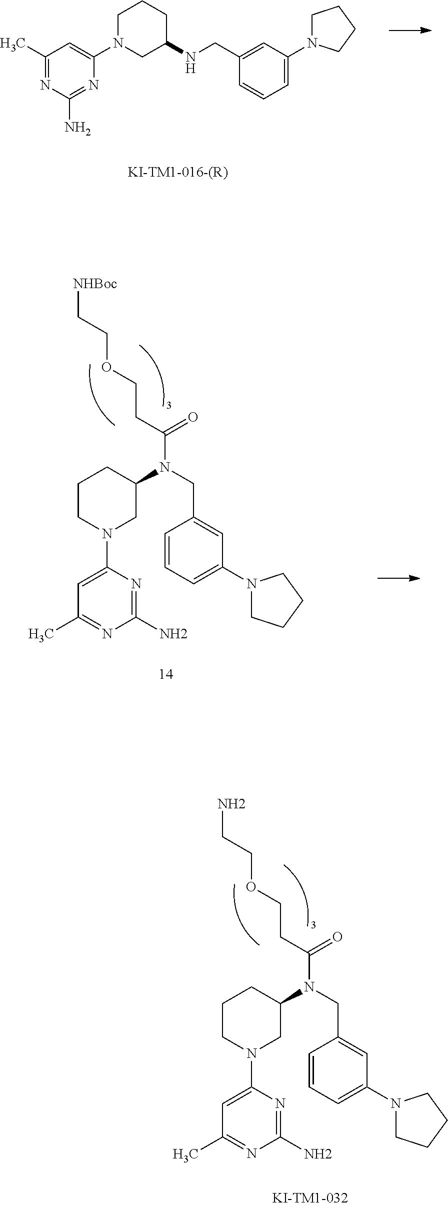

tert-Butyl (2-(1-(2-amino-6-methylpyrimidin-4-yl)piperidin-3-yl)-3-oxo-1-(3-(pyrrolidin-1-yl)phenyl)-6,9,12-trioxa-2-azatetradecan-14-yl)carbamate (14): Compound 14 was synthesized in a manner analogous to that used to prepare compound 13 using 2,2-dimethyl-4-oxo-3,8,11,14-tetraoxa-5-azaheptadecan-17-oic acid (61.3 mg, 0.16 mmol, 1.0 equiv.), N,N′-dicyclohexylcarbodiimide (33.4 mg, 0.16 mmol, 1.0 equiv.), DCM (0.22 M), 1-hydroxybenzotriazole (27.0 mg, 0.20 mmol, 1.2 equiv.) and DMF (0.73 M) as well as KI-TM-001 (60.0 mg, 0.16 mmol, 1.0 equiv.) and DCM (1.1 M). The title compound was isolated as a colorless oil (55.2 mg, 0.08 mmol)—51% yield

N-(1-(2-Amino-6-methylpyrimidin-4-yl)piperidin-3-yl)-3-(2-(2-(2-aminoethoxy)ethoxy)-ethoxy)-N-(3-(pyrrolidin-1-yl)benzyl)propenamide (KI-TM1-32): A vial was charged compound (14) 45.1 mg, 0.07 mmol, 1.0 equiv.) followed by 1:1 TFA/DCM (0.2 M). The reaction was allowed to sit at room temperature for 30 hours, and the volatiles were then removed in vacuo. The residue was then evaporated from DCM thrice and MeOH once. The crude product was purified via flash chromatography using 0/99/1%→15/84/1% MeOH/DCM/NH₃ as eluent. The title compound was isolated as a white solid (33.4 mg, 0.06 mmol)—87% yield. ¹H (CD₃OD, 500 MHz) δ 7.21-7.07 (m, 1H), 6.58-6.42 (m, 3H), 5.99 (s, 0.5H), 5.83 (s, 0.5H), 4.69 (dd, J=16.7, 4.7 Hz, 1H), 4.59 (t, J=15.2 Hz, 1H), 4.50 (d, J=11.9 Hz, 1H), 4.33 (d, J=12.5 Hz, 1H), 4.17-4.03 (m, 1H), 3.93-3.82 (m, 1H), 3.82-3.74 (m, 1H), 3.62 (qdd, J=8.8, 5.9, 3.5 Hz, 8H), 3.53 (dd, J=11.3, 6.0 Hz, 2H), 3.36-3.33 (m, 1H), 3.30-3.16 (m, 5H), 3.06 (t, J=11.8 Hz, 1H), 2.92-2.80 (m, 3H), 2.79-2.62 (m, 3H), 2.17-2.11 (m, 3H), 2.06-1.98 (m, 4H), 1.97-1.82 (m, 2H), 1.74 (d, J=11.0 Hz, 1H), 1.55-1.45 (m, 1H).

### N-(2-(2-(2-(2-Aminoethoxy)ethoxy)ethoxy)ethyl)-4-methyl-6-(4-((3-(pyrrolidin-1-yl)benzyl)amino)piperidin-1-yl)pyrimidin-2-amine (KI-TM1-041)

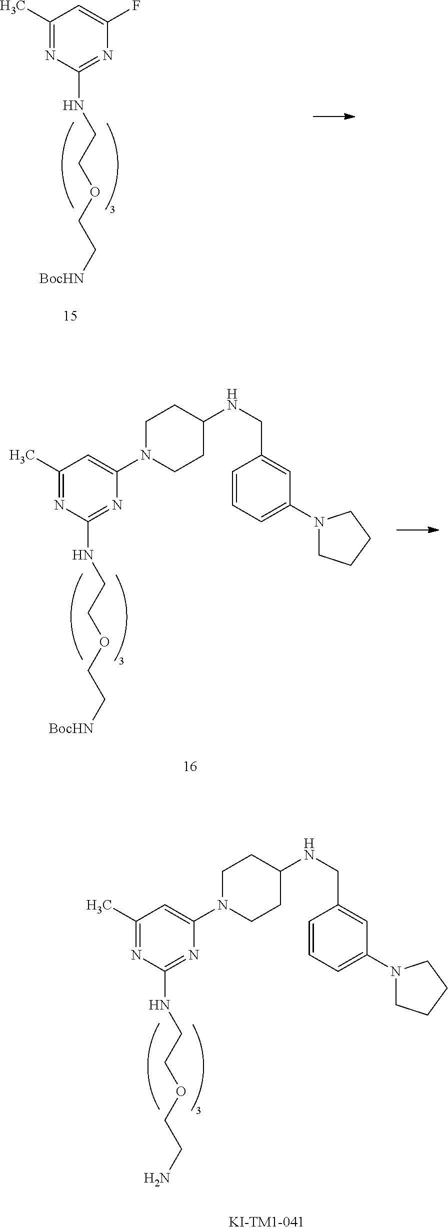

tert-Butyl (2-(2-(2-(2-((4-fluoro-6-methylpyrimidin-2-yl)amino)ethoxy)ethoxy)ethoxy)-ethyl)carbamate (15): A vial was charged with 2,4-difluoro-6-methylpyrimidine (100 mg, 0.76 mmol, 1.0 equiv.), tert-butyl (2-(2-(2-(2-aminoethoxy)ethoxy)ethoxy)ethyl)-carbamate (223 mg, 0.76 mmol, 1.0 equiv.), 0.13 mL (0.76 mmol, 1.0 equiv) of N,N-diisopropylethylamine, THF (0.76 M) at 0° C. The resulting mixture was stirred at room temperature and monitored by TLC. After reaction completion, the mixture was diluted with DCM and aqueous saturated sodium carbonate solution and extracted with DCM. The combined organic layers were washed with Brine. The crude product was purified via flash chromatography using 0/99/1%→15/84/1% MeOH/DCM/NH₃ as eluent. The title compound was isolated as a white powder (103 mg, 0.26 mmol)—34% yield. ¹H ((CD₃)₂SO) δ 6.75 (t, J=5.2 Hz, 1H), 6.37 (s, 2H), 3.52 (s, 3H), 3.51-3.45 (m, 6H), 3.37 (d, J=6.1 Hz, 3H), 3.30 (s, 4H), 3.17 (d, J=5.3 Hz, 1H), 3.06 (q, J=6.0 Hz, 2H), 1.37 (s, 9H).

tert-Butyl (2-(2-(2-(2-((4-methyl-6-(4-((3-(pyrrolidin-1-yl)benzyl)amino)piperidin-1-yl)pyrimidin-2-yl)amino)ethoxy)ethoxy)ethoxy)ethyl)carbamate (16): A vial was charged with compound 15 (20.1 mg, 0.05 mmol, 1.0 equiv.), 4-((3-(pyrrolidin-1-yl)benzyl)amino)piperidin-1-ium·2 TFA (48.9 mg, 0.10 mmol, 2.0 equiv), caesium carbonate (57.0 mg, 0.18 mmol, 3.5 equiv) and THF (0.03 M). The resulting mixture was stirred at 110° C. for 48 h. After reaction completion, the mixture was cooled to room temperature, diluted with EtOAc, washed with H₂O (2×) and Brine. The crude product was purified via flash chromatography using 0/99/1%→15/84/1% MeOH/DCM/NH₃ as eluent. The title compound was isolated as a colorless oil (10.0 mg, 0.02 mmol)—30% yield. ¹H (CD₃OD, 500 MHz) δ 7.19-7.11 (m, 1H), 6.67-6.56 (m, 2H), 6.50 (dd, J=8.1, 2.0 Hz, 1H), 4.75 (s, 1H), 4.25 (d, J=13.3 Hz, 1H), 3.80-3.75 (m, 2H), 3.68-3.59 (m, 9H), 3.54-3.46 (m, 3H), 3.42 (t, J=5.4 Hz, 2H), 3.31-3.26 (m, 5H), 3.23 (t, J=5.6 Hz, 2H), 2.83-2.72 (m, 3H), 2.07-2.01 (m, 4H), 1.99-1.94 (m, 2H), 1.45 (s, 8H).

N-(2-(2-(2-(2-Aminoethoxy)ethoxy)ethoxy)ethyl)-4-methyl-6-(4-((3-(pyrrolidin-1-yl)benzyl)amino)piperidin-1-yl)pyrimidin-2-amine (KI-TM1-041): A vial was charged compound (16) (10.0 mg, 0.02 mmol, 1.0 equiv.) followed by 1:1 TFA/DCM (0.2 M). The reaction was allowed to sit at room temperature for 30 hours, and the volatiles were then removed in vacuo. The residue was then evaporated from DCM thrice and MeOH once. The crude product was purified via flash chromatography using 0/99/1%→15/84/1% MeOH/DCM/NH₃ as eluent. The title compound was isolated as a white solid (7.6 mg, 0.01 mmol)—89% yield. ¹H (CD₃OD, 500 MHz) δ 7.32-7.22 (m, 1H), 6.78-6.65 (m, 4H), 4.21 (d, J=4.7 Hz, 2H), 3.99-3.86 (m, 1H), 3.76-3.62 (m, 15H), 3.50-3.42 (m, 2H), 3.17-3.11 (m, 3H), 3.09-2.94 (m, 2H), 2.28 (d, J=10.3 Hz, 2H), 2.10-2.01 (m, 6H), 1.64 (ddd, J=24.6, 12.4, 4.3 Hz, 2H).

### 2-(2-((6-((1-(2-Amino-6-methylpyrimidin-4-yl)piperidin-4-yl)(3-(pyrrolidin-1-yl)benzyl)amino)hexyl)oxy)ethoxy)-N-(2-(2,6-dioxopiperidin-3-yl)-1,3-dioxoisoindolin-4-yl)acetamide (KI-TM1-044)

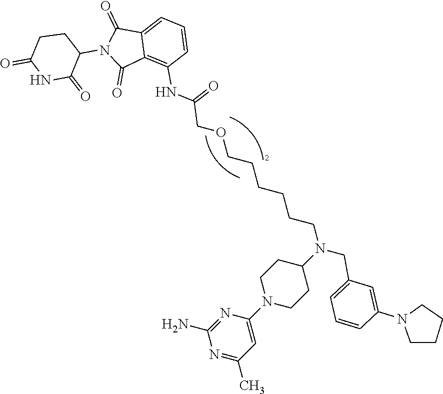

2-(2-((6-((1-(2-Amino-6-methylpyrimidin-4-yl)piperidin-4-yl)(3-(pyrrolidin-1-yl)benzyl)amino)hexyl)oxy)ethoxy)-N-(2-(2,6-dioxopiperidin-3-yl)-1,3-dioxoisoindolin-4-yl)acetamide (KI-TM1-044): A vial was charged with KI-TM-001 (13.3 mg, 0.04 mmol, 1.0 equiv.), N-(2-(2,6-dioxopiperidin-3-yl)-1,3-dioxoisoindolin-4-yl)-2-(2-((5-iodopentyl)oxy)-ethoxy)-acetamide (25.0 mg, 0.04 mmol, 3.0 equiv.), 7.61 μL (0.06 mmol, 1.5 equiv) of triethylamine, DMF (0.08 M) at 40° C. and monitored by TLC. After reaction completion, the mixture was diluted with EtOAc and water and extracted with EtOAc. The organic layer was washed with water. The crude product was purified via flash chromatography using 0/99/1%→10/89/1% MeOH/DCM/NH₃ as eluent. The title compound was isolated as a white solid (17.1 mg, 0.02 mmol)—53% yield. ¹H ((CD₃)₂SO) δ 11.96 (s, 1H), 11.15 (s, 1H), 10.36 (s, 1H), 8.73 (d, J=8.4 Hz, 1H), 7.85 (t, J=7.9 Hz, 1H), 7.63 (d, J=7.3 Hz, 1H), 7.05 (t, J=7.7 Hz, 1H), 6.59-6.49 (m, 2H), 6.36 (d, J=7.0 Hz, 1H), 5.76 (s, 1H), 5.16 (dd, J=12.8, 5.4 Hz, 1H), 4.19 (s, 2H), 3.83-3.70 (m, 2H), 3.65-3.55 (m, 2H), 3.47 (s, 2H), 3.36-3.33 (m, 2H), 3.21-3.15 (m, 4H), 2.93-2.82 (m, 1H), 2.80-2.66 (m, 2H), 2.66-2.55 (m, 2H), 2.39-2.32 (m, 3H), 2.10 (s, 3H), 2.08-2.03 (m, 1H), 1.96-1.89 (m, 6H), 1.72 (d, J=11.3 Hz, 2H), 1.43-1.35 (m, 2H), 1.36-1.23 (m, 5H), 1.22-1.15 (m, 4H).

### 2-(1-(2-amino-6-methylpyrimidin-4-yl)piperidin-4-yl)-N-(2-(2,6-dioxopiperidin-3-yl)-1,3-dioxoisoindolin-4-yl)-1-(3-(pyrrolidin-1-yl)phenyl)-9,12,15-trioxa-2-azahenicosan-21-amide (KI-TM1-043)

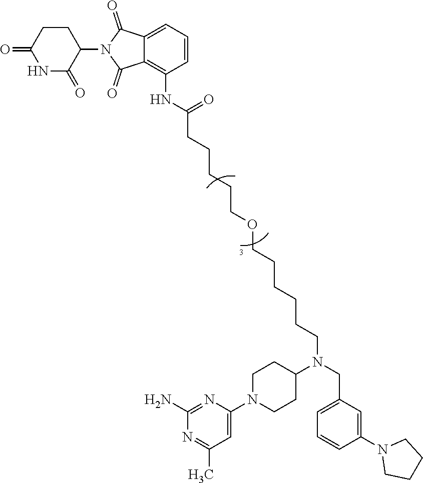

2-(1-(2-amino-6-methylpyrimidin-4-yl)piperidin-4-yl)-N-(2-(2,6-dioxopiperidin-3-yl)-1,3-dioxoisoindolin-4-yl)-1-(3-(pyrrolidin-1-yl)phenyl)-9,12,15-trioxa-2-azahenicosan-21-amide (KI-TM1-043): A vial was charged with KI-TM-001 (15.6 mg, 0.04 mmol, 1.0 equiv.), N-(2-(2,6-Dioxopiperidin-3-yl)-1,3-dioxoisoindolin-4-yl)-6-(2-(2-((6-iodohexyl)oxy)ethoxy)ethoxy)hexanamide (25.0 mg, 0.04 mmol, 3.0 equiv.), 7.61 μL (0.06 mmol, 1.5 equiv) of triethylamine, DMF (0.08 M) at 40° C. and monitored by TLC. After reaction completion, the mixture was diluted with EtOAc and water and extracted with EtOAc. The organic layer was washed with water. The crude product was purified via flash chromatography using 0/99/1%→10/89/1% MeOH/DCM/NH₃ as eluent. The title compound was isolated as a yellow solid (16.0 mg, 0.02 mmol)—43% yield. ¹H ((CD₃)₂SO) δ 11.15 (s, 1H), 8.48 (d, J=8.3 Hz, 1H), 7.89 (d, J=8.7 Hz, 1H), 7.83 (t, J=7.9 Hz, 1H), 7.61 (d, J=7.3 Hz, 1H), 7.12-6.97 (m, 2H), 6.88-6.79 (m, 1H), 6.59-6.48 (m, 2H), 6.36 (d, J=7.8 Hz, 1H), 5.15 (dd, J=12.9, 5.4 Hz, 1H), 4.38 (s, 2H), 3.60-3.35 (m, 15H), 3.25-3.13 (m, 5H), 2.99-2.83 (m, 2H), 2.74 (s, 3H), 2.67-2.61 (m, 3H), 2.48-2.34 (m, 5H), 2.09 (s, J=17.2 Hz, 3H), 2.01-1.86 (m, 4H), 1.79-1.68 (m, 2H), 1.67-1.56 (m, 2H), 1.56-1.48 (m, 2H), 1.47-1.38 (m, 3H), 1.38-1.30 (m, 5H), 1.30-1.17 (m, 6H).

### MYB Assays

An assay using a 5×Myb-response element (MRE)-luciferase construct was used to evaluate the compounds' ability to bind MYB and/or disrupt the MYB-NFIB interaction. The 5×MRE reporter was conducted in both HEK293T cells and Jurkat cells, and for the HEK293T system a transient transfection of plasmid vectors expressing either MYB or MYB-NFIB, and a plasmid containing the 5×MRE promoter sequence cloned upstream of Firefly luciferase were used. For the Jurkat system, the reporter was driven by endogenous MYB, and the 5×MRE construct was transiently transfected. Cells were treated with two different high concentrations of each compound (10 and 20 M) and the luciferase activity was measured 18 hours post-treatment. Individual wells were normalized for viability (cell count) using the Cyto-tox One assay (Promega).

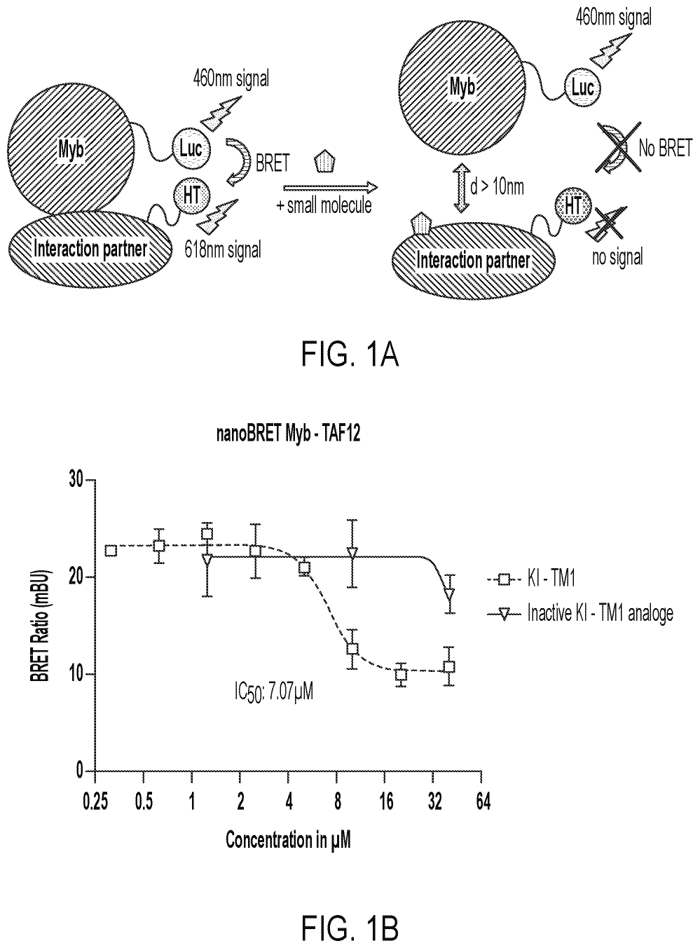

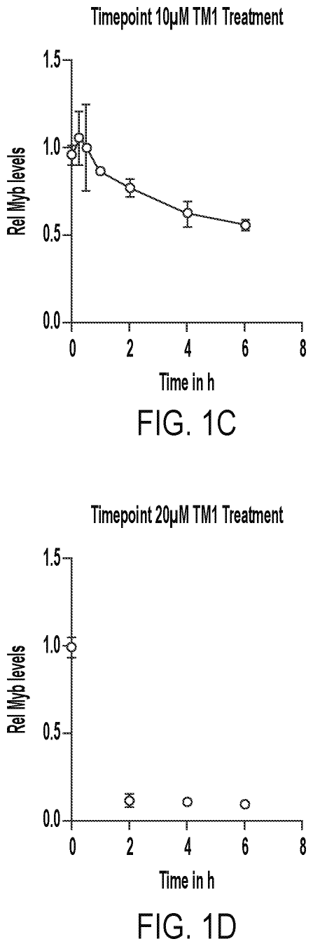

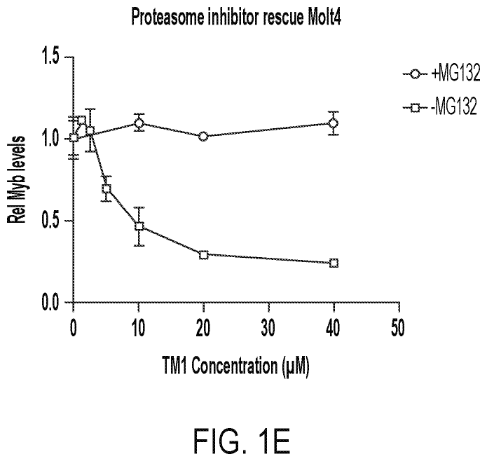

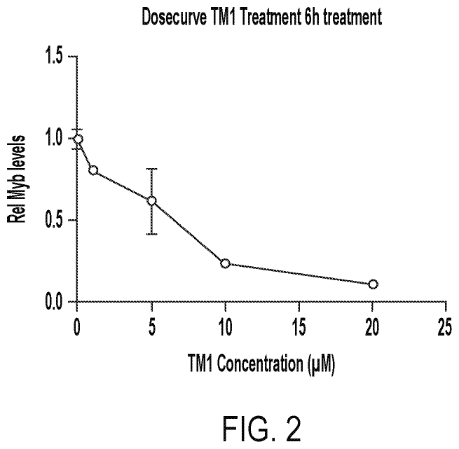

In-cell bioluminescence resonance energy transfer (BRET) assays were developed to assess the influence of the compounds on MYB-p300 and MYB-TAF12 interactions (FIG. 1A). KI-TM-001 inhibited the Myb-TAF12 interaction with an IC₅₀ value of 7.1 μM (FIG. 1B). A transcriptionally inactive analog of KI-TM-001 did not block the interaction, consistent with the notion that this interaction is critical for transcription. The ability of KI-TM-001 to modulate MYB protein and transcript levels in MOLT4 and Jurkat cells was also evaluated. KI-TM-001 impacts protein levels rapidly, within 2 hours, and in a dose-dependent manner (FIG. 1C-D; FIG. 2). Degradation appears to be proteasome-dependent as MYB can be rescued by treatment with the proteasome inhibitor MG132.

Efforts have also included assessment of impact on viable cell growth using traditional luminescent cell viability assays (e.g., CellTiter-Glo™ or GTG) and high-throughput profiling in barcoded and pooled cancer cell lines. Collaboration with the Broad Institute's PRISM cell profiling platform provided the ability to survey sensitivity to KI-TM-001 against 800 cancer cell lines representing a broad spectrum of lineages, including 300 suspension lines and 500 adherent lines.

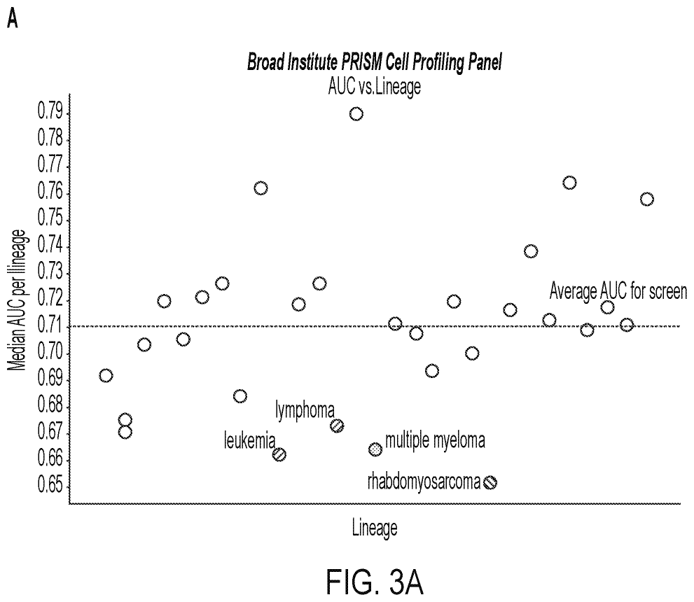

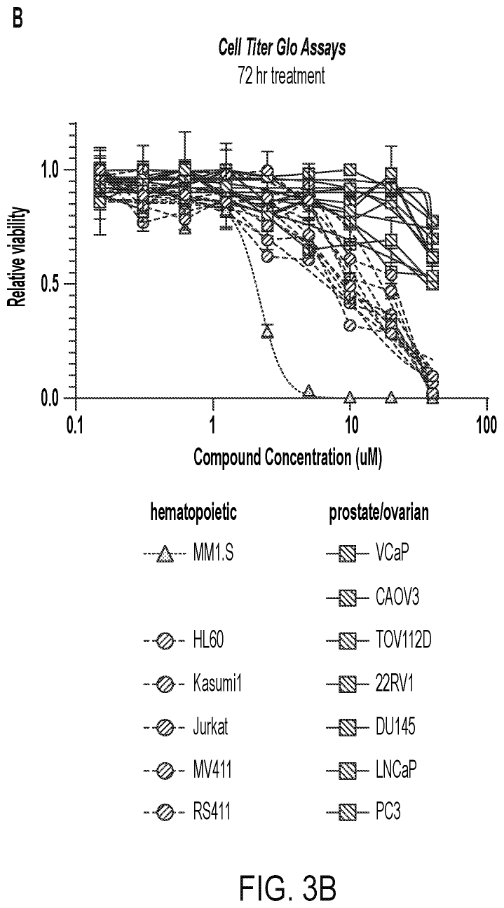

Roughly 650 of the lines passed quality control analyses. It is rare to observe lineage enrichment for compounds with potencies greater than 1 μM in transcription assays. However, the data set suggested mild enrichment for leukemia, lymphoma, multiple myeloma and rhabdomyosarcoma (RMS) cell lines (FIG. 3A). Moreover, a number of hematopoietic lines with IC50 values under 1 μM from this data set were observed and are currently being evaluated using CTG assays. For example, the APL line NB4 had an IC₅₀ of 210 nM. The CTG assay is well established has been used to survey viability of cell lines used routinely in the lab, noting a separation between solid tumor lines and hematopoetic lineages (FIG. 3B).

Thus, compounds of Formula (I) are potent modulators of MYB-mediated transcription and disruptors of critical protein-protein interactions (Table 1).

### Reporter Assay Procedures

HEK293T cells were plated at a density of 7500 cells/well in a 384-well plate. The following day, the cells were transfected with plasmids encoding 1)c-Myb under control of a CMV promoter, 2) 5×MRE reporter construct driving firefly luciferase expression in a 1:2 ratio. 4 hours following transfection, compound analogs were added using a Tecan transfer tool to the wells. The plate was incubated for 18 hours, and then firefly luciferase signal measured using promega Dual-Glo luciferase assay system according to the manufacturer's protocols. The signal was normalized to control wells which were treated with DMSO vehicle.

Jurkat cells were reverse transfected at a density of 40,000 cells/well at the time of plating with a plasmid encoding 5×MRE reporter construct driving firefly luciferase expression. 4 hours following transfection, the compound analogs were added using a Tecan transfer tool to the wells. The plate was incubated for 18 hours, and then firefly luciferase signal was measured using promega Steady-glo luciferase assay system according to the manufacturer's protocols. The signal was normalized to control wells which were treated with DMSO vehicle.

### Cell-Titer Glo Assay Procedure

HEK293T cells were plated at a density of 5000 cells/well in a 384-well plate. The following day, compound analogs were added using a Tecan transfer tool. The plates were incubated for 3 days, and then the viability was measured using promega Cell-titer glo assay according to the manufacturer's protocols.

TABLE 1

| | IC₅₀ | | |
|---|---|---|---|
| | | | Cell Titer Glow |
| | Jurkat Reporter | HEK293T | Viability Jurkat |
| Compound Structure & Name | (μM) | Reporter (μM) | (μM) |
| 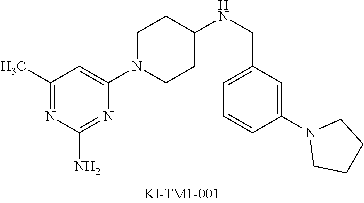 [SMILES: Cc1cc(N2CCC(NCc3cccc(N4CCCC4)c3)CC2)nc(N)n1] | 3.49 | 15.88 | 3.24 |
| 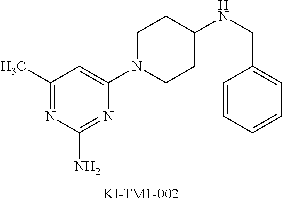 [SMILES: Cc1cc(N2CCC(NCc3ccccc3)CC2)nc(N)n1] | 25.67 | >40 | >30 |
| 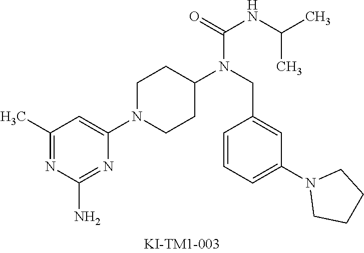 [SMILES: Cc1cc(N2CCC(N(Cc3cccc(N4CCCC4)c3)C(=O)NC(C)C)CC2)nc(N)n1] | 26.41 | Not measured | >30 |
| 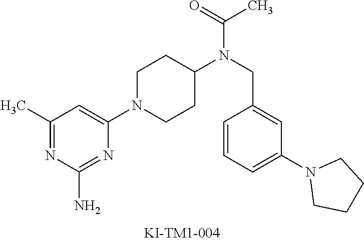 [SMILES: CC(=O)N(Cc1cccc(N2CCCC2)c1)C1CCN(c2cc(C)nc(N)n2)CC1] | 10.98 | Not measured | >30 |
| 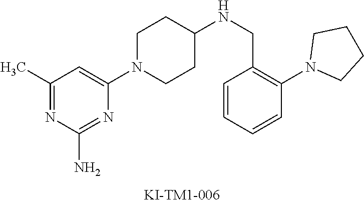 [SMILES: Cc1cc(N2CCC(NCc3ccccc3N3CCCC3)CC2)nc(N)n1] | 3.09 | >40 | >30 |
| 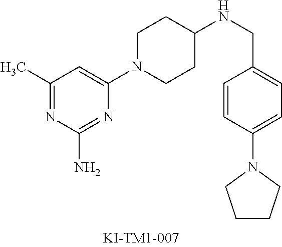 [SMILES: Cc1cc(N2CCC(NCc3ccc(N4CCCC4)cc3)CC2)nc(N)n1] | 17.34 | >40 | >30 |
| 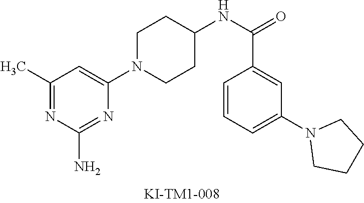 [SMILES: Cc1cc(N2CCC(NC(=O)c3cccc(N4CCCC4)c3)CC2)nc(N)n1] | 11.11 | >40 | >30 |
| 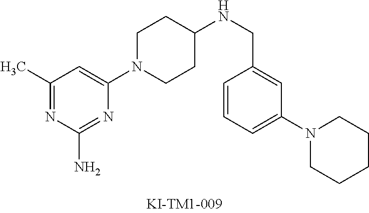 [SMILES: Cc1cc(N2CCC(NCc3cccc(N4CCCCC4)c3)CC2)nc(N)n1] | 4.18 | 25.25 | 11.16 |
| 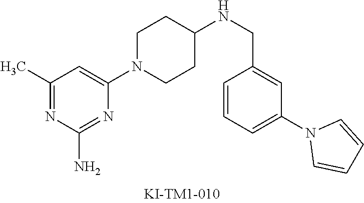 [SMILES: Cc1cc(N2CCC(NCc3cccc(-n4cccc4)c3)CC2)nc(N)n1] | 12.06 | 14.8 | 21.56 |
| 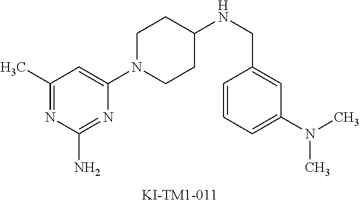 | >40 | >40 | >30 |
| 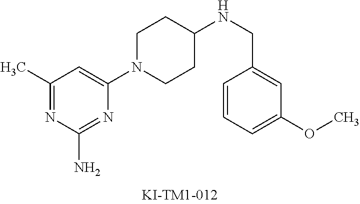 | 30.12 | >40 | >30 |
| 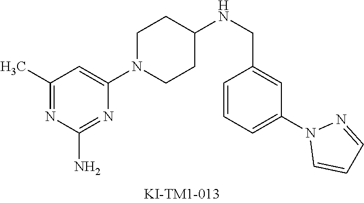 [SMILES: Cc1cc(N2CCC(NCc3cccc(-n4cccn4)c3)CC2)nc(N)n1] | 30.58 | >40 | >30 |
| 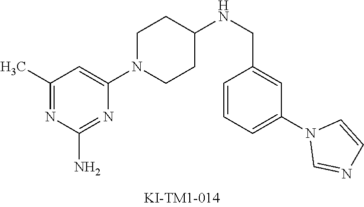 [SMILES: Cc1cc(N2CCC(NCc3cccc(-n4ccnc4)c3)CC2)nc(N)n1] | 25.74 | >40 | >30 |
| 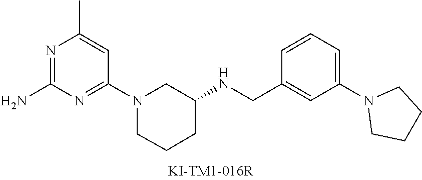 [SMILES: Cc1cc(N2CCC[C@@H](NCc3cccc(N4CCCC4)c3)C2)nc(N)n1] | 2.254 | Not measured | 4.41 |
| 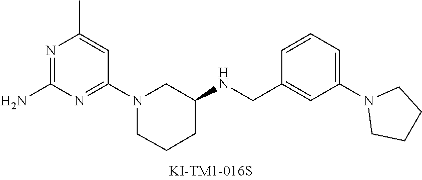 [SMILES: Cc1cc(N2CCC[C@H](NCc3cccc(N4CCCC4)c3)C2)nc(N)n1] | 8.076 | Not measured | 19.69 |
| 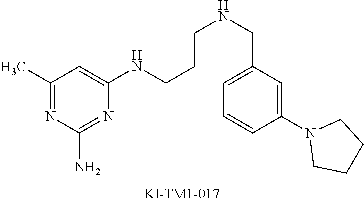 [SMILES: Cc1cc(NCCCNCc2cccc(N3CCCC3)c2)nc(N)n1] | 8.549 | Not measured | >30 |
| 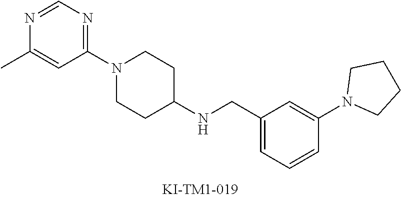 [SMILES: Cc1cc(N2CCC(NCc3cccc(N4CCCC4)c3)CC2)ncn1] | Not measured | >30 | Not measured |
| 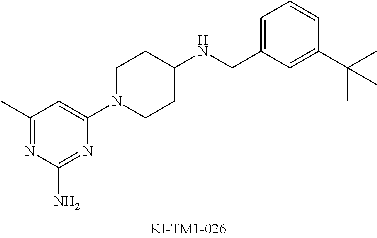 [SMILES: Cc1cc(N2CCC(NCc3cccc(C(C)(C)C)c3)CC2)nc(N)n1] | Not measured | >30 | Not measured |
| 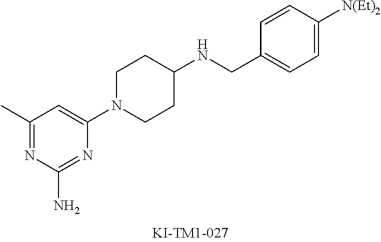 | Not measured | >30 | Not measured |

Additional assays were employed to evaluate the compounds of the disclosure, including Molt4 reporter and viability assays as described below and reported in Table 2.

### Cell Line Engineering

Molt4 cells were stably transduced with a lentiviral construct encoding a 5×MRE Myb-responsive promoter driving firefly luciferase expression. The reporter output is controlled by the levels/activity of endogenous Myb protein in the Molt4 cell line.

### Reporter Assay Procedure

Molt4-5×MRE-FLuc cells were plated at a density of 10000 cells/well in 25 uL of media in a 384-well plate. Compound analogs were added using a Tecan transfer tool to the wells. The plate was incubated for 4 hours, and then firefly luciferase signal measured using promega One-Glo luciferase assay system according to the manufacturer's protocols. The signal was normalized to control wells which were treated with DMSO vehicle.

### Cell-Titer Glo Assay Procedure

Molt4-5×MRE-FLuc cells were plated at a density of 10000 cells/well in 25 uL of media in a 384-well plate. The following day, compound analogs were added using a Tecan transfer tool. The plates were incubated for 4 hours, and then the viability was measured using promega Cell-titer glo assay according to the manufacturer's protocols.

TABLE 2

IC₅₀

| | Molt4-5xMRE- | Cell Titer Glo Molt4- |
|---|---|---|
| | FLuc Reporter | 5xMRE-FLuc Viability |
| Compound | (μM) | (μM) |
| KI-TM1-001 | 13.58 | >40 |
| KI-TM1-003 | >40 | >40 |
| KI-TM1-004 | >40 | >40 |
| KI-TM1-006 | >40 | >40 |
| KI-TM1-007 | >40 | >40 |
| KI-TM1-008 | >40 | >40 |
| KI-TM1-009 | 23.21 | >40 |
| KI-TM1-010 | 21.16 | >40 |
| KI-TM1-011 | >40 | >40 |
| KI-TM1-012 | >40 | >40 |
| KI-TM1-014 | >40 | >40 |
| KI-TM1-016R | >40 | >40 |
| KI-TM1-016S | 22.28 | >40 |
| KI-TM1-017 | >40 | >40 |
| KI-TM1-019 | 22.14 | >40 |
| KI-TM1-026 | >40 | >40 |
| KI-TM1-027 | >40 | >40 |
| KI-TM1-028 | >40 | >40 |
| KI-TM1-030 | 19.81 | >40 |
| KI-TM1-031 | >40 | >40 |
| KI-TM1-032 | >40 | >40 |
| KI-TM1-033 | 23.47 | >40 |
| KI-TM1-034 | >40 | >40 |
| KI-TM1-036 | 24.89 | >40 |
| KI-TM1-037 | >40 | >40 |
| KI-TM1-038 | >40 | >40 |
| KI-TM1-039 | >40 | >40 |
| KI-TM1-040 | >40 | >40 |
| KI-TM1-041 | >40 | >40 |
| KI-TM1-043 | >40 | >40 |
| KI-TM1-044 | >40 | >40 |

### REFERENCES

1. Ramsay R G, Gonda, T J. MYB function in normal and cancer cells. Nat Rev Cancer 2008; 8(7):523-534.
2. Lahortiga I, De Keersmaecker K, Van Vlierberghe P, et al. Duplication of the MYB oncogene in T cell acute lymphoblastic leukemia. Nat Genet 2007; 39(5):593-595.
3. Zuber J, Rappaport A R, Luo W, et al. An integrated approach to dissecting oncogene addiction implicates a Myb-coordinated self-renewal program as essential for leukemia maintenance. Genes Dev 2011; 25(15):1628-1640.
4. Pattabiraman D R, McGirr C, Shakhbazov K, et al. Interaction of c-Myb with p300 is required for the induction of acute myeloid leukemia (AML) by human AML oncogenes.

Blood 2014; 123:2682-2690.
5. Huang Y, Stiwala K, Bronstein J, et al. Identification and characterization of Hoxa9 binding sites in hematopoietic cells. Blood 2012; 119:388-398.
6. Uttaekar S, Dasse E, Coulibaly A, et al. Targeting acute myeloid leukemia via a small molecule inhibitor of the Myb/p300 interaction. Blood 2016; 127:1173-1182.
7. Bradner, J. E., McPherson, O. M. & Koehler, A. N. A method for the covalent capture and screening of diverse small molecules in a microarray format. Nature protocols 1, 2344-2352, doi:10.1038/nprot.2006.282 (2006).
8. Vegas, A. J., Bradner, J. E., Tang, W., McPherson, O. M., Greenberg, E. F., Koehler, A. N. & Schreiber, S. L. Fluorous-based small-molecule microarrays for the discovery of histone deacetylase inhibitors. Angewandte Chemie 46, 7960-7964, doi:10.1002/anie.200703198 (2007).
9. Leifer B S, Doyle S K, Richters A, Evans H L, Koehler A N. An array-based ligand discovery platform for proteins with short half-lives. Methods Enzymol 2018; 610:191-218
10. Mitani Y, Li J, Rao P H, et al. Comprehensive Analysis of the MYB-NFIB Gene Fusion in Salivary Adenoid Cystic Carcinoma: Incidence, Variability, and Clinicopathologic Significance. Clinical Cancer Research 2010:16:4722-4731.
11. Brayer, K. J., Frerich, C. A., Kang, H. N. & Ness, S. A. Recurrent Fusions in MYB and MYBL1 Define a Common, Transcription Factor-Driven Oncogenic Pathway in Salivary Gland Adenoid Cystic Carcinoma. Cancer Discovery 6, 176-187, doi:10.1158/2159-8290.cd-15-0859 (2016).
12. Coriano C, Powell E, Xu W. Monitoring Ligand-Activated Protein-Protein Interactions Using Bioluminescent Resonance Energy Transfer (BRET) Assay. Methods Mol Biol 2016; 1473: 3-15.
13. Xu Y, Milazzo J P, Somerville T D D, et al. A TFIID-SAGA perturbation that targets MYB and suppresses Acute Myeloid Leukemia. Cancer Cell 2018; 33(1):13-28.
14. Yu C, Mannan A M, Yvone G M, et al. High-throughput identification of genotype-specific cancer vulnerabilities in mixtures of barcoded tumor cell lines. Nat Biotechnology 2016; 34(4):419-423.
15. Kaspar P, Prochazka J, Efenberkova M, et al. c-Myb regulates tumorigenic potential of embryonal rhabdomyosarcoma cells. Sci Rep 2019; 9(1):6432.
16. Andersson M K, Aman P, Stenman G. IGF2/IGF1R Signaling as a Therapeutic Target in MYB-Positive Adenoid Cystic Carcinomas and Other Fusion Gene-Driven Tumors. Cells 2019; 8(8), E913.
17. Kaspar P, Zikova M, Bartunek P, et al. The expression of c-Myb correlates with the levels of rhabdomyosarcoma-specific marker myogenin. Sci Rep 2015; 15:15090
18. Kaspar, Petr, et al. “c-Myb regulates tumorigenic potential of embryonal rhabdomyosarcoma cells.” Scientific reports 9.1 (2019): 6342.
19. Andersson, Mattias K., Pierre Åman, and Göran Stenman. “IGF2/IGF1R Signaling as a Therapeutic Target in MYB-Positive Adenoid Cystic Carcinomas and Other Fusion Gene-Driven Tumors.” Cells 8.8 (2019): 913.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 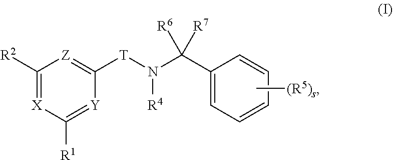
or a pharmaceutically acceptable salt, solvate, hydrate, polymorph, co-crystal, tautomer, stereoisomer, isotopically labeled derivative, or prodrug thereof, wherein:
X, Y, and Z are each independently N or CR³;
T is 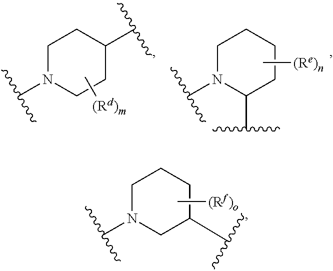
or —NR^{m}—(CR^{g}R^{h})₃—;
R¹, R², and R³ are each independently hydrogen, halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};
R⁴ is hydrogen, substituted or unsubstituted alkyl, —C(═O)OR^{A}, —C(═O)R^{A}, —C(═O)N(R^{A})₂, or a nitrogen protecting group;
each R⁵ is independently —OR^{A}, —N(R^{A})₂, substituted or unsubstituted alkyl, substituted or unsubstituted carbocyclyl, substituted or unsubstituted aryl, substituted or unsubstituted heterocyclyl, or substituted or unsubstituted heteroaryl;
R⁶ and R⁷ are each independently hydrogen, halogen, or substituted or unsubstituted alkyl, or R⁶ and R⁷ together with the carbon to which they are attached form a carbonyl;
each R^{A} is independently hydrogen, substituted or unsubstituted alkyl, substituted or unsubstituted heteroalkyl, substituted or unsubstituted acyl, an oxygen protecting group when bound to an oxygen, or a nitrogen protecting group when bound to a nitrogen, or two R^{A} groups are joined to form a substituted or unsubstituted heterocyclic ring or substituted or unsubstituted heteroaryl ring;
R^{d}, R^{e}, R^{f}, R^{g}, and R^{h} are each independently halogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —NO₂, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};
R^{m} is hydrogen, substituted or unsubstituted alkyl, —C(═O)OR^{A}, —C(═O)R^{A}, —C(═O)N(R^{A})₂, or a nitrogen protecting group;
m, n, and o are each independently 0, 1, 2, 3, 4, or 5; and
s is 0, 1, 2, 3, 4, or 5;
provided that the compound is not of formula: 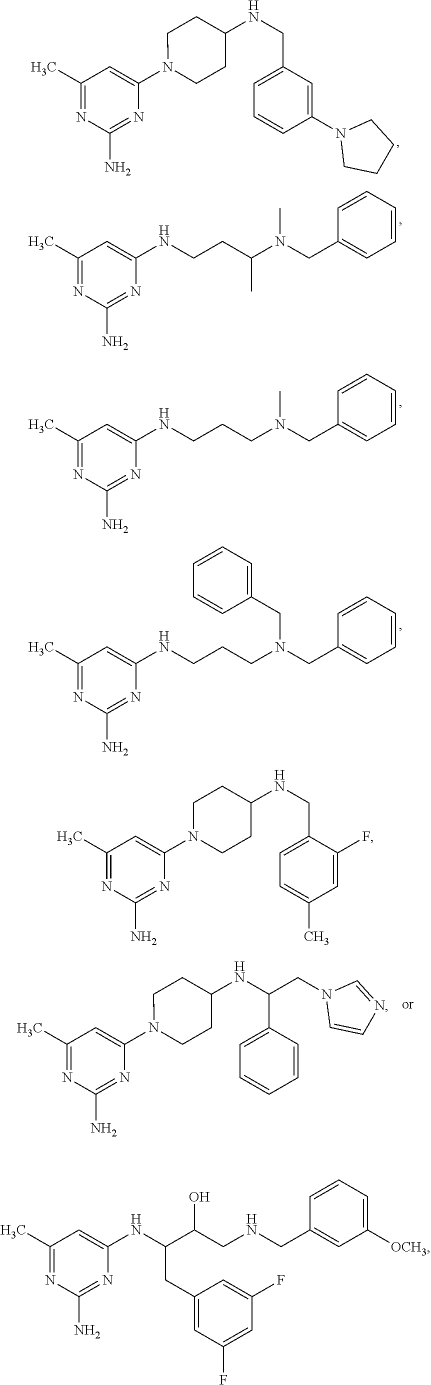
or a pharmaceutically acceptable salt, solvate, hydrate, polymorph, co-crystal, tautomer, stereoisomer, isotopically labeled derivative, or prodrug thereof.

2. The compound of claim 1, wherein the compound is of Formula: 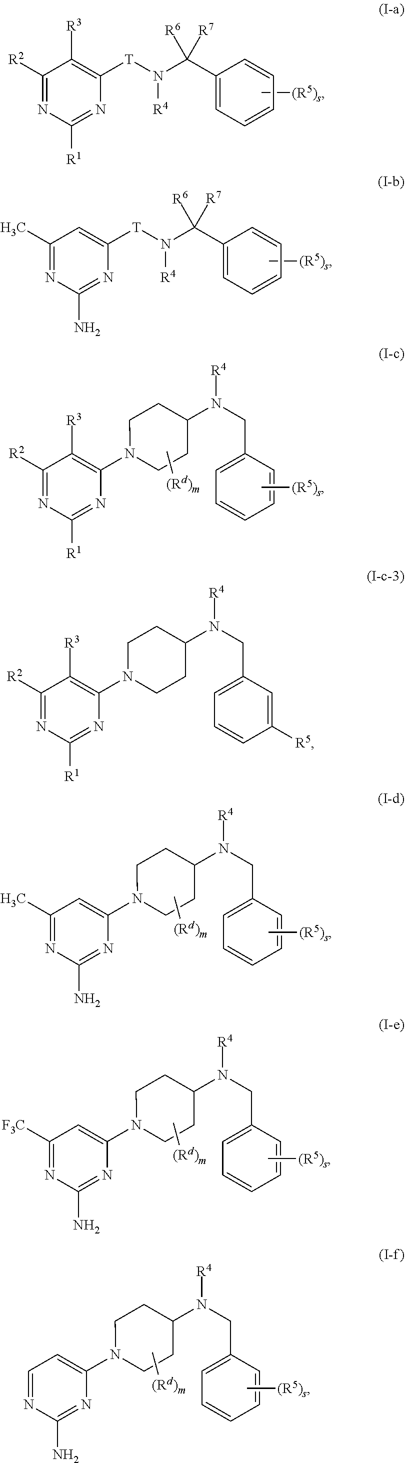 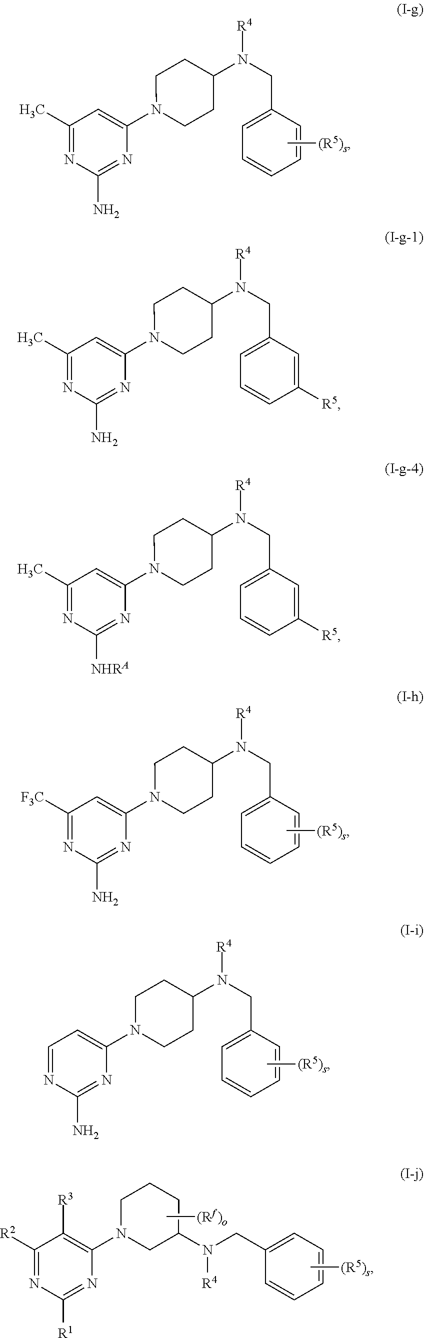 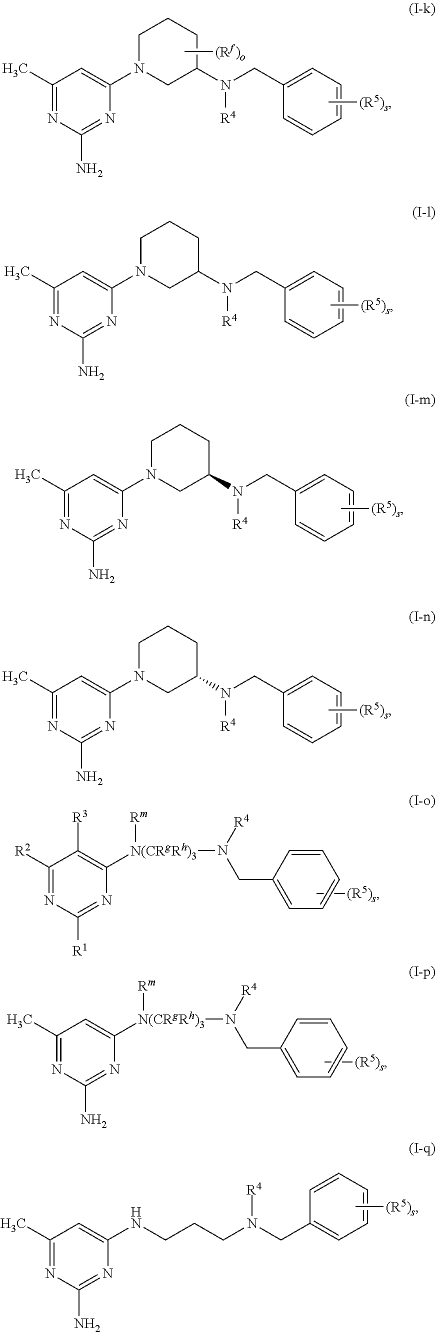 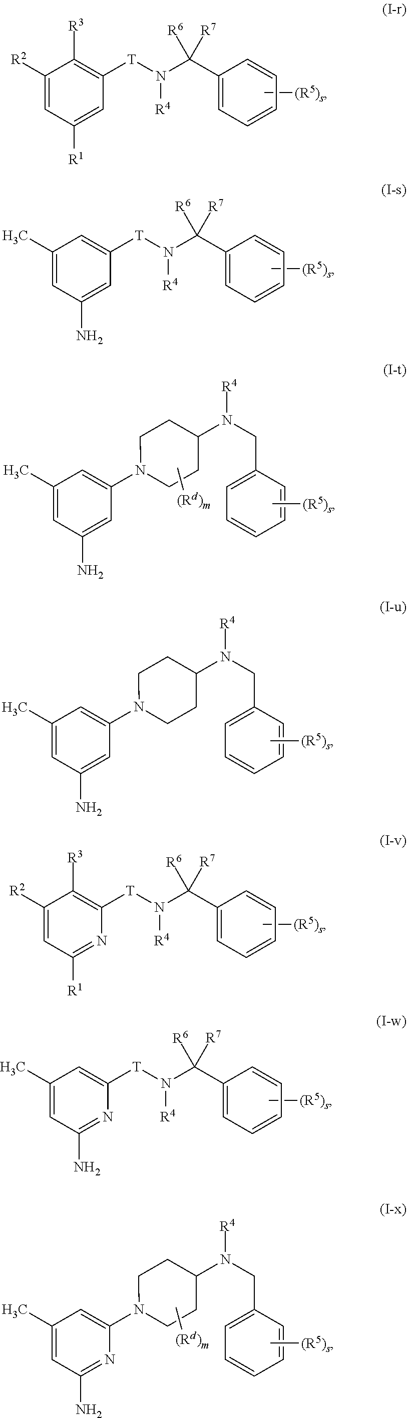 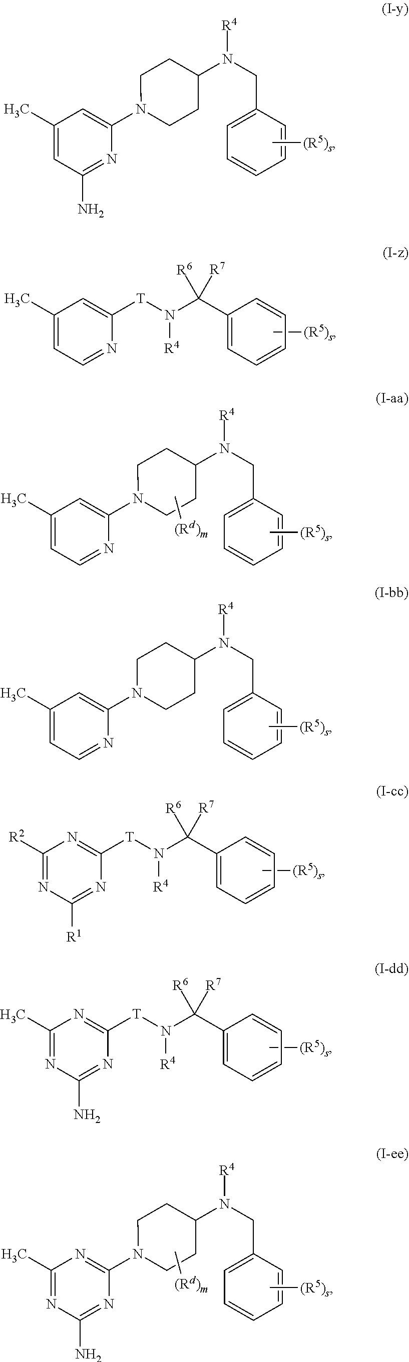 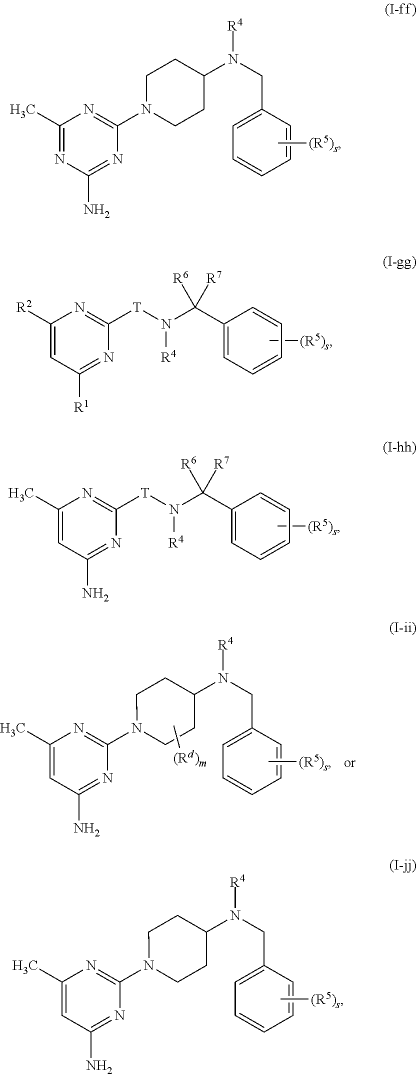
or a pharmaceutically acceptable salt, solvate, hydrate, polymorph, co-crystal, tautomer, stereoisomer, isotopically labeled derivative, or prodrug thereof.

3-44. (canceled)

45. The compound of claim 1, or a pharmaceutically acceptable salt, solvate, hydrate, polymorph, co-crystal, tautomer, stereoisomer, isotopically labeled derivative, or prodrug thereof, wherein X and Y are each N; and Z is CR³.

46-48. (canceled)

49. The compound of claim 1, or a pharmaceutically acceptable salt, solvate, hydrate, polymorph, co-crystal, tautomer, stereoisomer, isotopically labeled derivative, or prodrug thereof, wherein Y and Z are each N; and X is CH.

50. (canceled)

51. The compound of claim 1, or a pharmaceutically acceptable salt, solvate, hydrate, polymorph, co-crystal, tautomer, stereoisomer, isotopically labeled derivative, or prodrug thereof, wherein R¹, R², and R³ are each independently hydrogen, halogen, cyano, substituted or unsubstituted alkyl, —NO₂, —OR^{A}, —N(R^{A})₂, or —NR^{A}C(═O)R^{A}.

52-54. (canceled)

55. The compound of claim 1, or a pharmaceutically acceptable salt, solvate, hydrate, polymorph, co-crystal, tautomer, stereoisomer, isotopically labeled derivative, or prodrug thereof, wherein R¹ is —N(R^{A})₂.

56-69. (canceled)

70. The compound of claim 1, or a pharmaceutically acceptable salt, solvate, hydrate, polymorph, co-crystal, tautomer, stereoisomer, isotopically labeled derivative, or prodrug thereof, wherein R² is hydrogen, unsubstituted alkyl, or haloalkyl.

71-77. (canceled)

78. The compound of claim 1, or a pharmaceutically acceptable salt, solvate, hydrate, polymorph, co-crystal, tautomer, stereoisomer, isotopically labeled derivative, or prodrug thereof, wherein R³ is hydrogen or substituted or unsubstituted alkyl.

79. (canceled)

80. The compound of claim 1, or a pharmaceutically acceptable salt, solvate, hydrate, polymorph, co-crystal, tautomer, stereoisomer, isotopically labeled derivative, or prodrug thereof, wherein R⁴ is hydrogen, substituted alkyl, —C(═O)OR^{A}, —C(═O)R^{A}, —C(═O)N(R^{A})₂, or a nitrogen protecting group.

81-101. (canceled)

102. The compound of claim 1, or a pharmaceutically acceptable salt, solvate, hydrate, polymorph, co-crystal, tautomer, stereoisomer, isotopically labeled derivative, or prodrug thereof, wherein each R⁵ is independently —OR^{A}, —N(R^{A})₂, substituted or unsubstituted heterocyclyl, or substituted or unsubstituted heteroaryl.

103-104. (canceled)

105. The compound of claim 1, or a pharmaceutically acceptable salt, solvate, hydrate, polymorph, co-crystal, tautomer, stereoisomer, isotopically labeled derivative, or prodrug thereof, wherein R⁵ is 5- to 6-membered heterocyclyl or 5- to 6-membered heteroaryl.

106-136. (canceled)

138. The compound of claim 1, or a pharmaceutically acceptable salt, solvate, hydrate, polymorph, co-crystal, tautomer, stereoisomer, isotopically labeled derivative, or prodrug thereof, wherein T is:

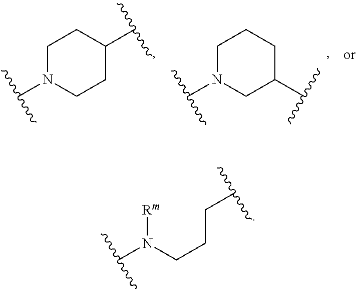

139-140. (canceled)

141. The compound of claim 1, or a pharmaceutically acceptable salt, solvate, hydrate, polymorph, co-crystal, tautomer, stereoisomer, isotopically labeled derivative, or prodrug thereof, wherein T is:

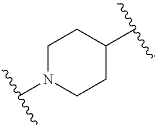 [SMILES: *C1CCN(*)CC1]

142-143. (canceled)

144. The compound of claim 1, or a pharmaceutically acceptable salt, solvate, hydrate, polymorph, co-crystal, tautomer, stereoisomer, isotopically labeled derivative, or prodrug thereof, wherein s is 0 or 1.

145-146. (canceled)

147. The compound of claim 1, wherein the compound is of formula: 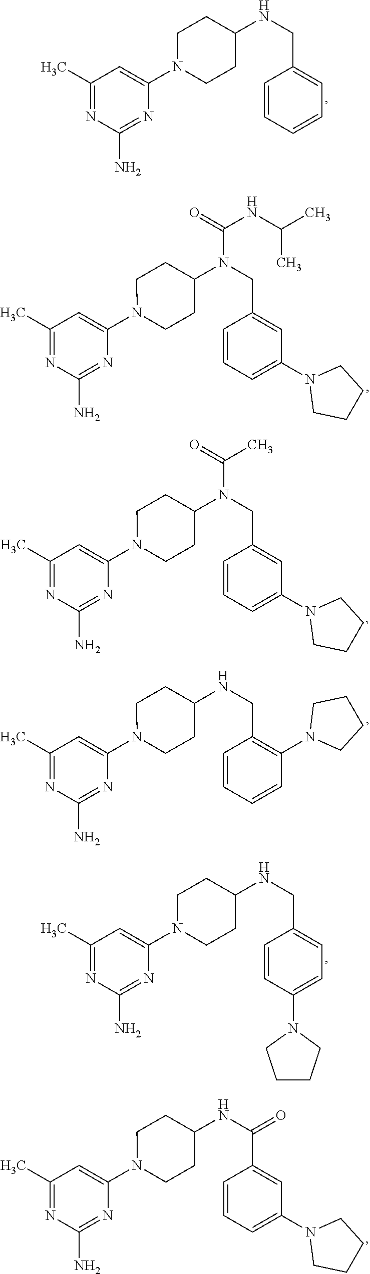 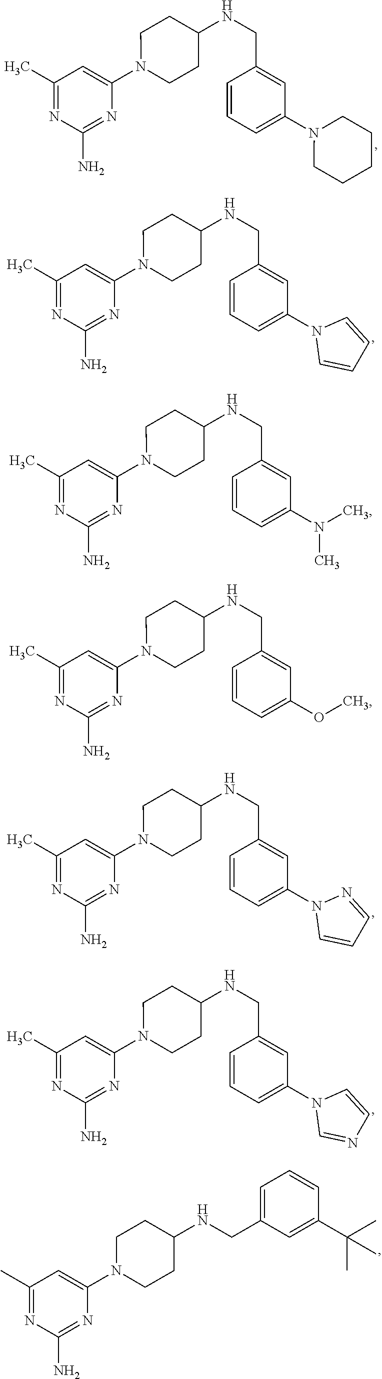 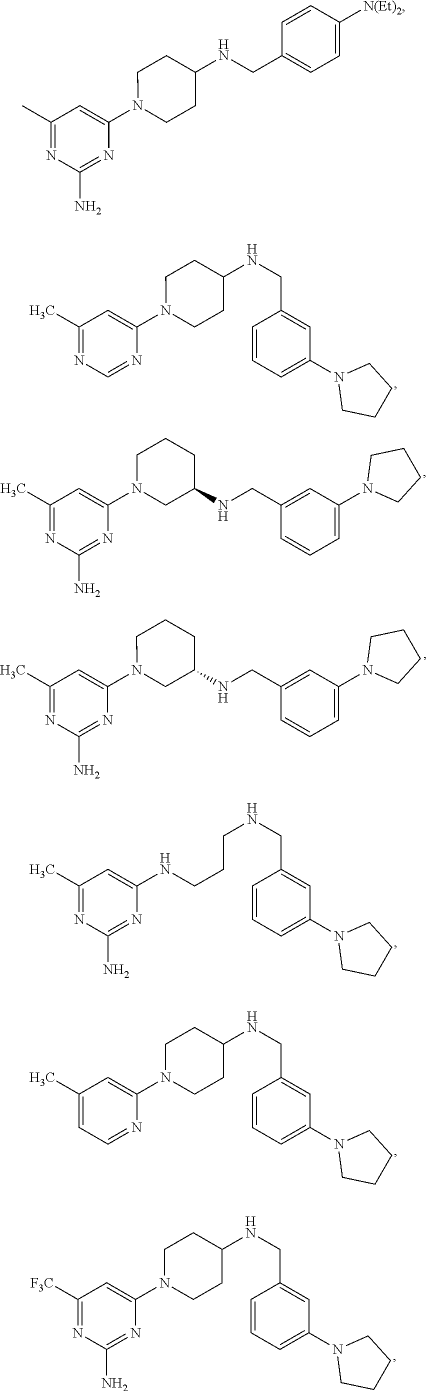 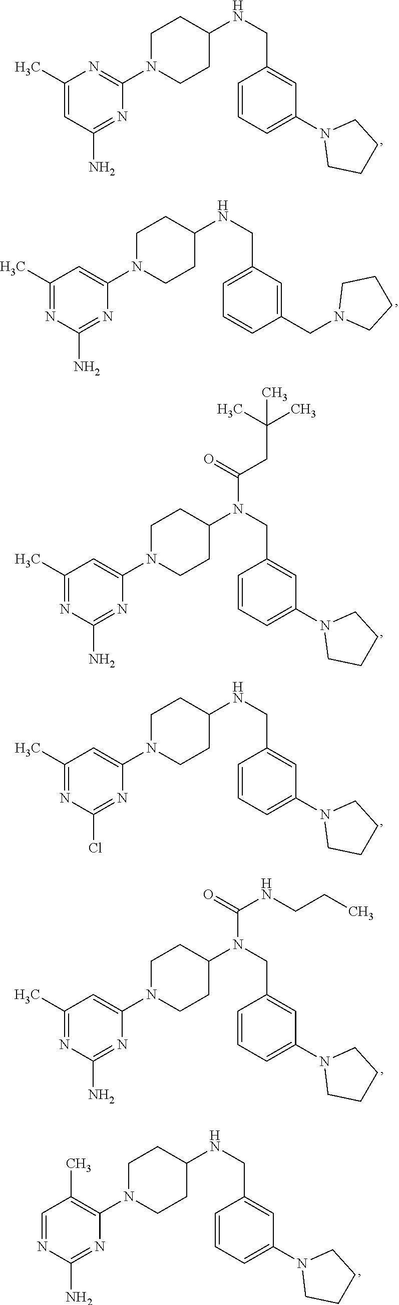 [SMILES: Cc1cc(N)nc(N2CCC(NCc3cccc(N4CCCC4)c3)CC2)n1 ; Cc1cc(N2CCC(NCc3cccc(CN4CCCC4)c3)CC2)nc(N)n1 ; Cc1cc(N2CCC(N(Cc3cccc(N4CCCC4)c3)C(=O)CC(C)(C)C)CC2)nc(N)n1 ; Cc1cc(N2CCC(NCc3cccc(N4CCCC4)c3)CC2)nc(Cl)n1 ; CCCNC(=O)N(Cc1cccc(N2CCCC2)c1)C1CCN(c2cc(C)nc(N)n2)CC1 ; Cc1cnc(N)nc1N1CCC(NCc2cccc(N3CCCC3)c2)CC1] 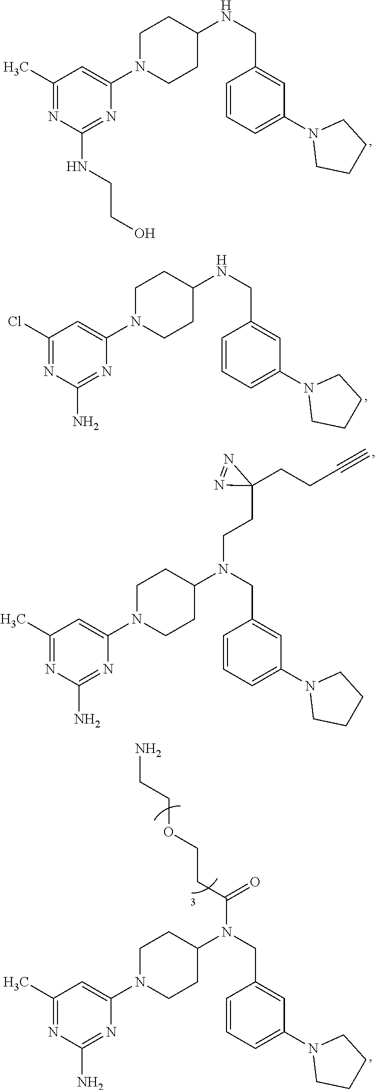 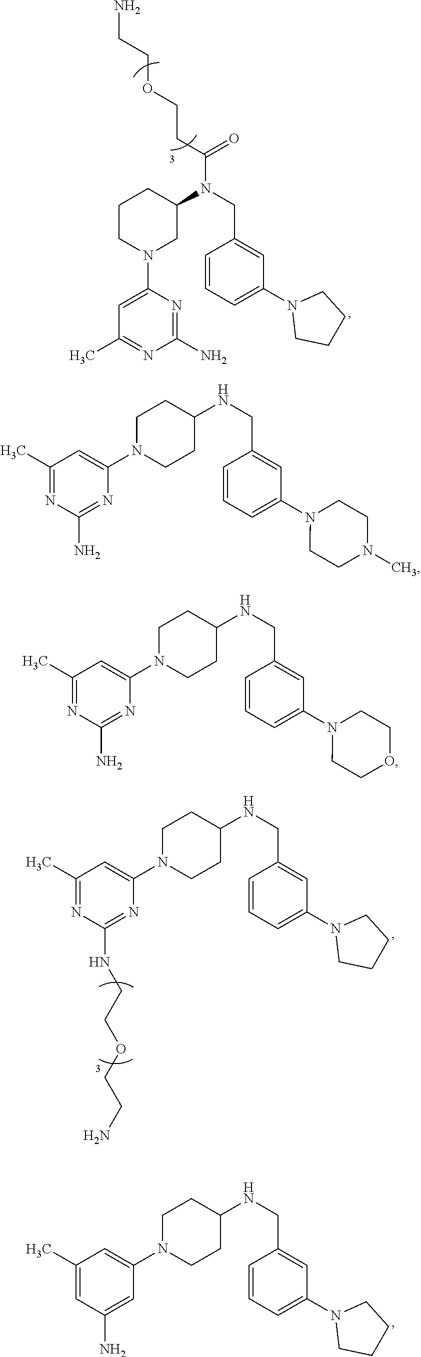 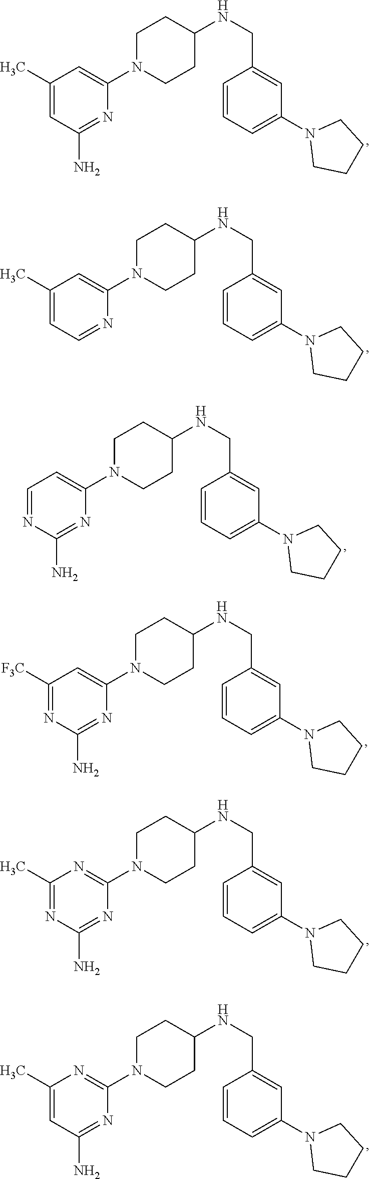 [SMILES: Cc1cc(N)nc(N2CCC(NCc3cccc(N4CCCC4)c3)CC2)c1 ; Cc1ccnc(N2CCC(NCc3cccc(N4CCCC4)c3)CC2)c1 ; Nc1nccc(N2CCC(NCc3cccc(N4CCCC4)c3)CC2)n1 ; Nc1nc(N2CCC(NCc3cccc(N4CCCC4)c3)CC2)cc(C(F)(F)F)n1 ; Cc1nc(N)nc(N2CCC(NCc3cccc(N4CCCC4)c3)CC2)n1 ; Cc1cc(N)nc(N2CCC(NCc3cccc(N4CCCC4)c3)CC2)n1] 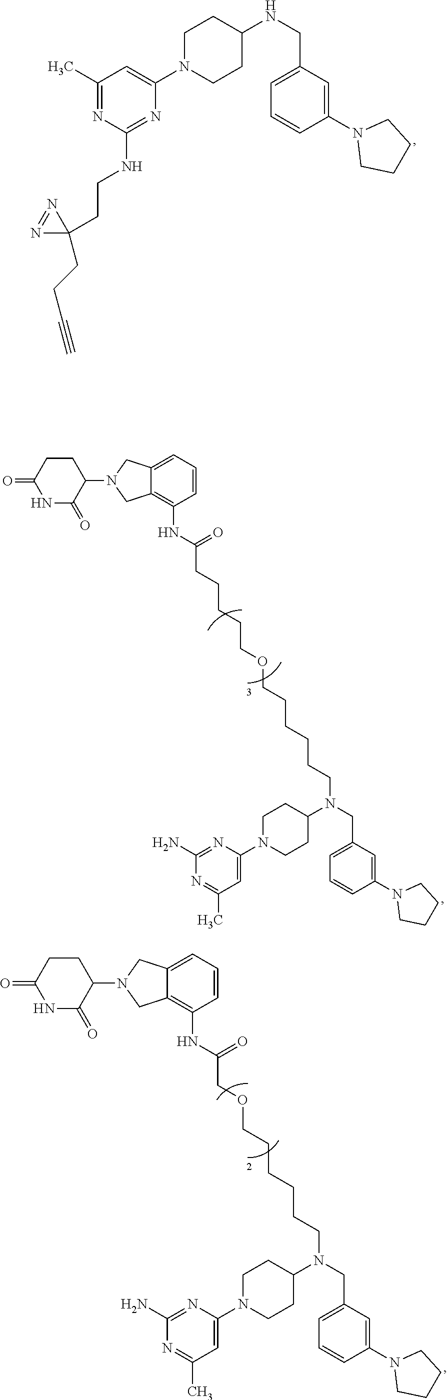
or a pharmaceutically acceptable salt, solvate, hydrate, polymorph, co-crystal, tautomer, stereoisomer, isotopically labeled derivative, or prodrug thereof.

148-151. (canceled)

152. A pharmaceutical composition comprising a compound of claim 1, or a pharmaceutically acceptable salt, solvate, hydrate, polymorph, co-crystal, tautomer, stereoisomer, isotopically labeled derivative, or prodrug thereof, and a pharmaceutically acceptable excipient.

153. A method of treating cancer in a subject in need thereof, the method comprising administering a compound of claim 1, or a pharmaceutically acceptable salt, solvate, hydrate, polymorph, co-crystal, tautomer, stereoisomer, isotopically labeled derivative, or prodrug thereof, to the subject.

154-157. (canceled)

158. A method of inhibiting MYB function, the method comprising contacting a compound of claim 1, or a pharmaceutically acceptable salt, solvate, hydrate, polymorph, co-crystal, tautomer, stereoisomer, isotopically labeled derivative, or prodrug thereof, with MYB or a protein associated with MYB.

159. (canceled)

160. A kit comprising a compound of claim 1, or a pharmaceutically acceptable salt thereof; and instructions for administering the compound, the pharmaceutically acceptable salt thereof, or the pharmaceutical composition to a subject.

161. A method of promoting the degradation of MYB or a protein associated with MYB (e.g., TAF12, p300, NFIB), the method comprising contacting MYB or a protein associated with MYB with a compound of claim 1, or a pharmaceutically acceptable salt thereof.

162-164. (canceled)
